(19) United States
(12) Patent Application Publication
(10) Pub. No.: US 2019/0062373 A1
(43) Pub. Date: Feb. 28, 2019

(54) METHOD OF GENERATING INTERACTING PEPTIDES

(71) Applicant: Peption, LLC, San Diego, CA (US)

(72) Inventor: Chang-Ho Baek, San Diego, CA (US)

(21) Appl. No.: 16/118,337

(22) Filed: Aug. 30, 2018

Related U.S. Application Data
(60) Provisional application No. 62/552,272, filed on Aug. 30, 2017.
(60) Provisional application No. 62/553,757, filed on Sep. 1, 2017.

(51) Int. Cl. C07K 7/06; C07K 7/08

(52) U.S. Cl. CPC C07K 7/06; C07K 7/08

(57) ABSTRACT

Disclosed herein is a method of designing small peptides for interacting with, binding to, or modulating the activity of, known protein or peptides. Further disclosed herein are methods for selecting sequences likely to have high binding activity against known protein sequences as well as peptides derived from the disclosed methods.

INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### REFERENCE TO SEQUENCE LISTING

The present application is being filed along with a Sequence Listing in electronic format. The Sequence Listing is provided as a file entitled PEPT_001A_SUBSTITUTE.TXT, created Nov. 13, 2018, which is 120 Kb in size. The information in the electronic format of the Sequence Listing is incorporated herein by reference in its entirety.

### BACKGROUND OF THE INVENTION

### Field

The present disclosure relates generally to the field of peptide design and protein-protein interactions.

### Background

Specific targeting of a protein by a select polypeptide sequence would be extremely useful in many branches of biotechnological sciences including disease prevention, diagnostics, and therapeutics. Animal-sourced antibodies are the present workhorse for detecting target proteins, however, production of these antibodies is tedious, time-consuming, and expensive. It would be highly desirable to develop synthetic antibodies (sAbs) that can be easily synthesized with low cost and time while retaining the favorable molecular recognition characteristics of the animal-sourced antibodies. In pursuit of this end, a number of approaches for predicting or identifying polypeptide sequences for said protein-protein interactions (PPI) have been developed. Computational prediction of PPIs utilizes a diverse database of known protein interactions, primary protein structures, associated physicochemical properties, and appearances of oligopeptide sequences for every protein encoded by the genome of an organism. However, these protein characteristics are not available for all proteins nor all organisms. Although massive library screening methods using the two-hybrid or phage display systems have been broadly accepted as key strategies to identify protein interaction partners, these approaches have been criticized for inaccurate results, and high labor requirements. The protein chip or microarray, another promising method, provides large-scale in vitro PPI data that could be used to identify target binder(s), and chips that expose precisely arranged spots of peptides on a solid support constitute an alternative to the current model. Each of these approaches has unique strengths and weaknesses regarding important factors of PPI such as coverage (library size), binding specificity, identification, experimental bias, post-translational modification, cost, and labor. However, none of these approaches provides a general pairing rule for protein-protein, protein-peptide, or peptide-peptide interaction.

The existence of amino acid complementarity would provide an important insight into protein folding and PPI. There currently are three approaches for formulating amino acid complementarity: 1) The hydropathic complementarity principle (molecular recognition theory); 2) The Root-Bernstein approach, where peptides complementary to a given sequence are encoded by antisense strand read in parallel to the sense strand; and 3) Approaches based on the periodicity of the genetic code.

The hydropathic complementarity principle is closely connected to the concept of sense-antisense peptide interaction, and states that amino acids encoded by the sense strand of DNA are complemented by amino acids with opposite hydropathic scores, coded by the standard 5′→3′ reading of the antisense strand. However, the hydropathic nature of sense and antisense peptides is determined mainly by the central bases of the corresponding codon triplets, and therefore is independent of the direction of the frame reading.

The Root-Bernstein approach suggests that complementary amino acid pairs may result from the parallel reading of complementary DNA strands (i.e. when sense strand is read in 5′-3′ direction, antisense strand is read in 3′→5′ direction). In this approach, it is believed that, of the 210 possible amino acid pairs of the standard 20 amino acids, no more than 26 could meet the physicochemical criteria for probable amino acid pairing. In fact, only 14 of these pairs were found to be genetically encoded pairs using the parallel reading approach. The other 12 pairings were found to be derivatives of the coded pairings in which a single base of the codon triplet had been varied.

In the approaches based on the periodicity of the genetic code, corresponding equivalent codons are categorized into two families of adenine/uracil (A/U) and cytosine/guanine (C/G) based on their central bases. In equivalent codons, the first two nucleotide bases of the triplets are complementary in parallel (3′→5′), with the third being the same. Because of the lack of complementarity with respect to the third base of the codons, peptides designed using this theory cannot be called true “antisense peptides.” The 3′→5′ reading of the complementary DNA strand strongly reduces the impact of the degeneracy of the genetic code on the number of amino acid complements. Thus, there are only minor differences in the assignments of the complementary amino acids according to the various existing approaches. Collectively, it is worth noting that all three approaches share identical complementary amino acid pairing partners for 14 out of 20 standard amino acids.

For all three approaches, successful instances of the complementary peptide-antipeptide interactions have been reported. However, these results have been controversial due to logical contradictions and the inability to repeat some of the studies. These doubts are exacerbated by the low stability of peptide-antipeptide complexes, with most interacting complements possessing dissociation constants (K_{d}) in the milli- to micromolar range). Furthermore, the sites of many peptide-antipeptide interactions haven't been precisely evaluated with careful attention to important factors including secondary structure, adjacent peptide sequences, amino acid turns in given peptide sequences, protein folding, and composition/spacing of the complementary amino acid pairings. Therefore, it is currently impossible to conclude which of the three approaches outlined above is most effective in predicting peptide-antipeptide interactions. Although various computer programs and publications for designing complementary peptides based on the sense strand of DNA or the resultant amino acid sequence have shown their feasibility, none provides a highly reliable algorithm for designing complementary peptide sequence that can interact with a preselected target peptide sequence with high affinity and specificity, comparable to traditional animal-sourced antibodies. Thus, there is a need for systems and methods that can take advantage of more of the diversity of interactions between amino acids. The present disclosure provides methods of designing binding peptides that go far beyond the limited set of amino acid interactions that could be predicted using previous methods. Further, while methods exist for screening libraries of random peptides for binding to a target protein, none of these methods allows the targeting of a specific region of a target protein, such as a particular region, binding site, or secondary structure element. Therefore, there is a need for methods that can specifically target regions, subsequences, or subdomains of a target protein. Accordingly, there is a need for a method to provide a general amino acid pairing rule for designing polypeptide synthetic antibody (sAb) sequences to interact with a chosen polypeptide sequence in any given target protein.

### SUMMARY

Disclosed herein is a molecular complex comprising a polypeptide configured to interact with a known binding partner wherein said polypeptide has a polypeptide sequence of between 6 and 20 amino acids in length, wherein said polypeptide sequence is composed by the steps of identifying the sequence of a binding partner; identifying 20% or more of the residues in the sequence of said binding partner; and, for each of the identified residues within the binding partner sequence, selecting the residue at the corresponding position for inclusion in the sequence of said polypeptide sequence as follows: where the identified residue within the binding partner sequence is Phe, the residue at the corresponding position for inclusion in the sequence of said polypeptide sequence is Lys or Glu; where the identified residue within the binding partner sequence is Leu, the residue at the corresponding position for inclusion in the sequence of said polypeptide sequence is Gln, Lys, or Glu; where the identified residue within the binding partner sequence is Ser, the residue at the corresponding position for inclusion in the sequence of said polypeptide sequence is Arg, Gly, Thr, or Ala; where the identified residue within the binding partner sequence is Thr, the residue at the corresponding position for inclusion in the sequence of said polypeptide sequence is Ser, Gly, Cys, or Arg; where the identified residue within the binding partner sequence is Tyr, the residue at the corresponding position for inclusion in the sequence of said polypeptide sequence is Ile or Val; where the identified residue within the binding partner sequence is Cys, the residue at the corresponding position for inclusion in the sequence of said polypeptide sequence is Thr or Ala; where the identified residue within the binding partner sequence is Trp, the residue at the corresponding position for inclusion in the sequence of said polypeptide sequence is Pro; where the identified residue within the binding partner sequence is Ile, the residue at the corresponding position for inclusion in the sequence of said polypeptide sequence is Asn, Asp, or Tyr; where the identified residue within the binding partner sequence is Met, the residue at the corresponding position for inclusion in the sequence of said polypeptide sequence is His; where the identified residue within the binding partner sequence is Asn, the residue at the corresponding position for inclusion in the sequence of said polypeptide sequence is Ile or Val; where the identified residue within the binding partner sequence is Lys, the residue at the corresponding position for inclusion in the sequence of said polypeptide sequence is Phe or Leu; where the identified residue within the binding partner sequence is Arg, the residue at the corresponding position for inclusion in the sequence of said polypeptide sequence is Thr, Ala, Ser, or Pro; where the identified residue within the binding partner sequence is Pro, the residue at the corresponding position for inclusion in the sequence of said polypeptide sequence is Arg, Gly, or Trp; where the identified residue within the binding partner sequence is His, the residue at the corresponding position for inclusion in the sequence of said polypeptide sequence is Met or Val; where the identified residue within the binding partner sequence is Gln, the residue at the corresponding position for inclusion in the sequence of said polypeptide sequence is Leu; where the identified residue within the binding partner sequence is Val, the residue at the corresponding position for inclusion in the sequence of said polypeptide sequence is Asn, Asp, Tyr, or His; where the identified residue within the binding partner sequence is Ala, the residue at the corresponding position for inclusion in the sequence of said polypeptide sequence is Ser, Gly, Cys, or Arg; where the identified residue within the binding partner sequence is Asp, the residue at the corresponding position for inclusion in the sequence of said polypeptide sequence is Ile or Val; where the identified residue within the binding partner sequence is Glu, the residue at the corresponding position for inclusion in the sequence of said polypeptide sequence is Phe or Leu; and where the identified residue within the binding partner sequence is Gly, the residue at the corresponding position for inclusion in the sequence of said polypeptide sequence is Thr, Ala, Ser, or Pro.

Disclosed herein is a method of making a polypeptide configured to interact with a known binding partner wherein said polypeptide has a polypeptide sequence of between 6 and 20 amino acids in length; wherein said polypeptide sequence is assembled by the steps of: (a) identifying the sequence of said binding partner; (b) identifying 20% or more of the residues in said binding partner sequence; and, (c) for each of the identified residues within the binding partner sequence, selecting the corresponding residue for inclusion in the sequence of said polypeptide sequence according to the relationships disclosed herein.

According to the methods and compositions disclosed herein, the selected residues for inclusion in the polypeptide sequence may occur at one of every two positions in the polypeptide sequence, at every other position in the polypeptide sequence, at one of every three positions in the polypeptide sequence, at every third position in the polypeptide sequence, at two of every three positions in the polypeptide sequence, or at 1, 2, or 3 of every four residues in the polypeptide sequence.

Also disclosed herein are binding peptides made according to the methods described herein, and conjugates and fusions thereof. Such conjugates or fusions may comprise a functional moiety, which may comprise one or more of a polypeptide, a therapeutic molecule, a protein, a nucleic acid, or a diagnostic moiety. Said functional moiety may, for example, comprise one or more of a radiolabel, spin label, affinity tag, or fluorescent label, and may comprise a linker, which may be a peptide, and may have the sequence GSGS (SEQ ID NO: 1), (G)ₙ (SEQ ID NO: 2), (GS)ₙ (SEQ ID NO: 3), (GGSGG)ₙ (SEQ ID NO: 4), (GGGS)ₙ (SEQ ID NO: 5), CYPEN (SEQ ID NO: 6), or KTGEVNN (SEQ ID NO: 7) or the like. Binding peptides designed according to the methods and compositions of the present disclosure may comprise one or more of the sequences LEQIKRLF (SEQ ID NO: 8), LLQVDVILL (SEQ ID NO: 9), LLQVDVILLCYPENLEQIKIRLF (SEQ ID NO: 10), LLQVDVILLCYPENLEQIKIRLFGSGSHHHHHH (SEQ ID NO: 11), EDRLQSYDLD (SEQ ID NO: 12), EDRLQSYDLDGSGSHHHHHH (SEQ ID NO: 13), ELDKAGFIKRQL (SEQ ID NO: 14), LEERGVKDRQLQ (SEQ ID NO: 15), LEILRAKDLALE (SEQ ID NO: 16), LEQIKIRLF (SEQ ID NO: 17), LSGLNEQRTQ (SEQ ID NO: 18), YDVDAIVPQC (SEQ ID NO: 19), CLTYDSHYLQ (SEQ ID NO: 20), LVAHVTSRKC (SEQ ID NO: 21), EYRLYLRALC (SEQ ID NO: 22), IEIVRKKPIF (SEQ ID NO: 23), IEIVRKKPIFC (SEQ ID NO: 24), CEDRLQSYDLD (SEQ ID NO: 25), EKLYLYYLQ (SEQ ID NO: 26), EKLYLYYLQC (SEQ ID NO: 27), LEQIKIRLFGSGSHHHHHH (SEQ ID NO: 28), LSRAYLSYEGSGSHHHHHH (SEQ ID NO: 29), EYRLYLRALCYPENLSRAYLSYEGSGSHHHHHH (SEQ ID NO: 30), DLDYAQLRDKCYPENEDRLQSYDLDGSGSHHHHHH (SEQ ID NO: 31), GKPIPNPLLGLDST (SEQ ID NO: 32), ELDKAGFIKRQLC (SEQ ID NO: 33), LLQVDVILLHHHHHHLEQIKIRLF (SEQ ID NO: 34), and/or CFFDSLVKQ (SEQ ID NO: 35).

In some embodiments, the methods and compositions disclosed herein comprise a molecular complex comprising a binding polypeptide configured to interact with a known binding partner where the binding polypeptide has a sequence of between 6 and 30 amino acids in length; and, where the binding polypeptide sequence is composed by the steps of identifying the sequence of said binding partner; and, identifying 20% or more of the residues in said binding partner sequence; and, for each of the identified residues within the binding partner sequence, selecting the residue at the corresponding position for inclusion in the sequence of said polypeptide sequence as follows: where the identified residue within the binding partner sequence is Phe, the residue at the corresponding position for inclusion in the sequence of said polypeptide sequence is Lys or Glu; where the identified residue within the binding partner sequence is Leu, the residue at the corresponding position for inclusion in the sequence of said polypeptide sequence is Gln, Lys, or Glu; where the identified residue within the binding partner sequence is Ser, the residue at the corresponding position for inclusion in the sequence of said polypeptide sequence is Arg, Gly, Thr, or Ala; where the identified residue within the binding partner sequence is Thr, the residue at the corresponding position for inclusion in the sequence of said polypeptide sequence is Ser, Gly, Cys, or Arg; where the identified residue within the binding partner sequence is Tyr, the residue at the corresponding position for inclusion in the sequence of said polypeptide sequence is Ile or Val; where the identified residue within the binding partner sequence is Cys, the residue at the corresponding position for inclusion in the sequence of said polypeptide sequence is Thr or Ala; where the identified residue within the binding partner sequence is Trp, the residue at the corresponding position for inclusion in the sequence of said polypeptide sequence is Pro; where the identified residue within the binding partner sequence is Ile, the residue at the corresponding position for inclusion in the sequence of said polypeptide sequence is Asn, Asp, or Tyr; where the identified residue within the binding partner sequence is Met, the residue at the corresponding position for inclusion in the sequence of said polypeptide sequence is His; where the identified residue within the binding partner sequence is Asn, the residue at the corresponding position for inclusion in the sequence of said polypeptide sequence is Ile or Val; where the identified residue within the binding partner sequence is Lys, the residue at the corresponding position for inclusion in the sequence of said polypeptide sequence is Phe or Leu; where the identified residue within the binding partner sequence is Arg, the residue at the corresponding position for inclusion in the sequence of said polypeptide sequence is Thr, Ala, Ser, or Pro; where the identified residue within the binding partner sequence is Pro, the residue at the corresponding position for inclusion in the sequence of said polypeptide sequence is Arg, Gly, or Trp; where the identified residue within the binding partner sequence is His, the residue at the corresponding position for inclusion in the sequence of said polypeptide sequence is Met or Val; where the identified residue within the binding partner sequence is Gln, the residue at the corresponding position for inclusion in the sequence of said polypeptide sequence is Leu; where the identified residue within the binding partner sequence is Val, the residue at the corresponding position for inclusion in the sequence of said polypeptide sequence is Asn, Asp, Tyr, or His; where the identified residue within the binding partner sequence is Ala, the residue at the corresponding position for inclusion in the sequence of said polypeptide sequence is Ser, Gly, Cys, or Arg; where the identified residue within the binding partner sequence is Asp, the residue at the corresponding position for inclusion in the sequence of said polypeptide sequence is Ile or Val; where the identified residue within the binding partner sequence is Glu, the residue at the corresponding position for inclusion in the sequence of said polypeptide sequence is Phe or Leu; where the identified residue within the binding partner sequence is Gly, the residue at the corresponding position for inclusion in the sequence of said polypeptide sequence is Thr, Ala, Ser, or Pro; and where the binding polypeptide may comprise part of a larger polypeptide.

In some embodiments, the methods and compositions disclosed herein comprise a method of making a polypeptide configured to interact with a known binding partner where the binding polypeptide has a sequence of between 6 and 20 amino acids in length; and, where the binding polypeptide sequence is assembled by the steps of: identifying the sequence of said binding partner; and, identifying 20% or more of the residues in said binding partner sequence; and, for each of the identified residues within the binding partner sequence, selecting the corresponding residue for inclusion in the sequence of said binding polypeptide sequence as follows: where the identified residue within the binding partner sequence is Phe, the residue at the corresponding position for inclusion in the sequence of said polypeptide sequence is Lys or Glu; where the identified residue within the binding partner sequence is Leu, the residue at the corresponding position for inclusion in the sequence of said polypeptide sequence is Gln, Lys, or Glu; where the identified residue within the binding partner sequence is Ser, the residue at the corresponding position for inclusion in the sequence of said polypeptide sequence is Arg, Gly, Thr, or Ala; where the identified residue within the binding partner sequence is Thr, the residue at the corresponding position for inclusion in the sequence of said polypeptide sequence is Ser, Gly, Cys, or Arg; where the identified residue within the binding partner sequence is Tyr, the residue at the corresponding position for inclusion in the sequence of said polypeptide sequence is Ile or Val; where the identified residue within the binding partner sequence is Cys, the residue at the corresponding position for inclusion in the sequence of said polypeptide sequence is Thr or Ala; where the identified residue within the binding partner sequence is Trp, the residue at the corresponding position for inclusion in the sequence of said polypeptide sequence is Pro; where the identified residue within the binding partner sequence is Ile, the residue at the corresponding position for inclusion in the sequence of said polypeptide sequence is Asn, Asp, or Tyr; where the identified residue within the binding partner sequence is Met, the residue at the corresponding position for inclusion in the sequence of said polypeptide sequence is His; where the identified residue within the binding partner sequence is Asn, the residue at the corresponding position for inclusion in the sequence of said polypeptide sequence is Ile or Val; where the identified residue within the binding partner sequence is Lys, the residue at the corresponding position for inclusion in the sequence of said polypeptide sequence is Phe or Leu; where the identified residue within the binding partner sequence is Arg, the residue at the corresponding position for inclusion in the sequence of said polypeptide sequence is Thr, Ala, Ser, or Pro; where the identified residue within the binding partner sequence is Pro, the residue at the corresponding position for inclusion in the sequence of said polypeptide sequence is Arg, Gly, or Trp; where the identified residue within the binding partner sequence is His, the residue at the corresponding position for inclusion in the sequence of said polypeptide sequence is Met or Val; where the identified residue within the binding partner sequence is Gln, the residue at the corresponding position for inclusion in the sequence of said polypeptide sequence is Leu; where the identified residue within the binding partner sequence is Val, the residue at the corresponding position for inclusion in the sequence of said polypeptide sequence is Asn, Asp, Tyr, or His; where the identified residue within the binding partner sequence is Ala, the residue at the corresponding position for inclusion in the sequence of said polypeptide sequence is Ser, Gly, Cys, or Arg; where the identified residue within the binding partner sequence is Asp, the residue at the corresponding position for inclusion in the sequence of said polypeptide sequence is Ile or Val; where the identified residue within the binding partner sequence is Glu, the residue at the corresponding position for inclusion in the sequence of said polypeptide sequence is Phe or Leu; where the identified residue within the binding partner sequence is Gly, the residue at the corresponding position for inclusion in the sequence of said polypeptide sequence is Thr, Ala, Ser, or Pro; and where the binding polypeptide may comprise part of a larger polypeptide.

In some embodiments, the methods and compositions disclosed herein comprise a method as described herein, where the selected corresponding residues for inclusion in the binding polypeptide sequence occur at one of every two positions in the binding polypeptide sequence. In some embodiments, the methods and compositions disclosed herein comprise a method as described herein, where the selected corresponding residues for inclusion in the binding polypeptide sequence occur at every other position in the binding polypeptide sequence. In some embodiments, the methods and compositions disclosed herein comprise a method as described herein, where the selected corresponding residues for inclusion in the binding polypeptide sequence occur at one of every three positions in the binding polypeptide sequence. In some embodiments, the methods and compositions disclosed herein comprise a method as described herein, where the selected corresponding residues for inclusion in the binding polypeptide sequence occur at every third position in the binding polypeptide sequence. In some embodiments, the methods and compositions disclosed herein comprise a method as described herein, where the selected corresponding residues for inclusion in the binding polypeptide sequence occur at two of every three positions in the binding polypeptide sequence. In some embodiments, the methods and compositions disclosed herein comprise a composition as described herein, where the selected corresponding residues for inclusion in the binding polypeptide sequence occur at one of every two positions in the binding polypeptide sequence. In some embodiments, the methods and compositions disclosed herein comprise a composition as described herein, where the selected corresponding residues for inclusion in the binding polypeptide sequence occur at every other position in the binding polypeptide sequence. In some embodiments, the methods and compositions disclosed herein comprise a composition as described herein, where the selected corresponding residues for inclusion in the binding polypeptide sequence occur at one of every three positions in the binding polypeptide sequence. In some embodiments, the methods and compositions disclosed herein comprise a composition as described herein, where the selected corresponding residues for inclusion in the binding polypeptide sequence occur at every third position in the binding polypeptide sequence. In some embodiments, the methods and compositions disclosed herein comprise a composition as described herein, where the selected corresponding residues for inclusion in the binding polypeptide sequence occur at two of every three positions in the binding polypeptide sequence. In some embodiments, the methods and compositions disclosed herein comprise a polypeptide made according to the method as described herein. In some embodiments, the methods and compositions disclosed herein comprise a polypeptide as described herein, which comprises a functional moiety. In some embodiments, the methods and compositions disclosed herein comprise a polypeptide as described herein where the functional moiety comprises one or more of a polypeptide, a therapeutic molecule, a protein, a nucleic acid, or a diagnostic moiety. In some embodiments, the methods and compositions disclosed herein comprise a polypeptide as described herein where the functional moiety comprises one or more of a radiolabel, spin label, affinity tag, or fluorescent label. In some embodiments, the methods and compositions disclosed herein comprise a polypeptide as described herein which comprises a linker. In some embodiments, the methods and compositions disclosed herein comprise a polypeptide as described herein where a linker is a peptide. In some embodiments, the methods and compositions disclosed herein comprise a polypeptide as described herein where the peptide includes the sequence GSGS (SEQ ID NO: 1), (G)n (SEQ ID NO: 2), (GS)n (SEQ ID NO: 3), (GGSGG)n (SEQ ID NO: 4), (GGGS)n (SEQ ID NO: 5), CYPEN (SEQ ID NO: 6), or KTGEVNN (SEQ ID NO: 7),In some embodiments, the methods and compositions disclosed herein comprise a binding polypeptide as described herein, where the binding polypeptide contains residues configured to interact with a second and optionally a third target protein in addition to the first target protein. In some embodiments, the methods and compositions disclosed herein comprise a binding polypeptide generated as described herein, where the binding polypeptide contains residues configured to interact with a second and optionally a third target protein in addition to the first target protein. In some embodiments, the methods and compositions disclosed herein comprise a fusion polypeptide, where the fusion comprises one or more binding polypeptides made according to the methods described herein. In some embodiments, the methods and compositions disclosed herein comprise a fusion polypeptide as described herein, where the fusion comprises 2, 3, 4, 5, or 6 binding polypeptides. In some embodiments, the methods and compositions disclosed herein comprise a molecular complex as disclosed herein, where said binding polypeptide is incorporated within a fusion polypeptide, and where said fusion comprises may further comprise one or more additional binding polypeptides. In some embodiments, the methods and compositions disclosed herein comprise a molecular complex as described herein, where the fusion polypeptide comprises 2, 3, 4, 5, or 6 binding polypeptides. In some embodiments, the methods and compositions disclosed herein comprise a binding polypeptide as described herein, where the sequence of the polypeptide comprises one or more of sequence LEQIKRLF (SEQ ID NO: 8), LLQVDVILL (SEQ ID NO: 9), LLQVDVILLCYPENLEQIKIRLF (SEQ ID NO: 10), LLQVDVILLCYPENLEQIKIRLFGSGSHHHHHH (SEQ ID NO: 11), EDRLQSYDLD (SEQ ID NO: 12), EDRLQSYDLDGSGSHHHHHH (SEQ ID NO: 13), ELDKAGFIKRQL (SEQ ID NO: 14), LEERGVKDRQLQ (SEQ ID NO: 15), LEILRAKDLALE (SEQ ID NO: 16), LEQIKIRLF (SEQ ID NO: 17), LSGLNEQRTQ (SEQ ID NO: 18), YDVDAIVPQC (SEQ ID NO: 19), CLTYDSHYLQ (SEQ ID NO: 20), LVAHVTSRKC (SEQ ID NO: 21), EYRLYLRALC (SEQ ID NO: 22), IEIVRKKPIF (SEQ ID NO: 23), IEIVRKKPIFC (SEQ ID NO: 24), CEDRLQSYDLD (SEQ ID NO: 25), EKLYLYYLQ (SEQ ID NO: 26), EKLYLYYLQC (SEQ ID NO: 27), LEQIKIRLFGSGSHHHHHH (SEQ ID NO: 28), LSRAYLSYEGSGSHHHHHH (SEQ ID NO: 29), EYRLYLRALCYPENLSRAYLSYEGSGSHHHHHH (SEQ ID NO: 30), DLDYAQLRDKCYPENEDRLQSYDLDGSGSHHHHHH (SEQ ID NO: 31), GKPIPNPLLGLDST (SEQ ID NO: 32), ELDKAGFIKRQLC (SEQ ID NO: 33), LLQVDVILLHHHHHHLEQIKIRLF (SEQ ID NO: 34), and/or CFFDSLVKQ (SEQ ID NO: 35). In some embodiments, the methods and compositions disclosed herein comprise a binding polypeptide as described herein, or a nucleic acid encoding said binding peptide, where the sequence of said polypeptide comprises one or more of the sequences provided in Table 6. In some embodiments, the methods and compositions disclosed herein comprise such a binding peptide, or a nucleic acid encoding such a binding peptide, where the sequence of the nucleic acid comprises one or more of the sequences provided in Table 7. In some embodiments, the methods and compositions disclosed herein comprise a method of making a binding polypeptide configured to interact with a known binding partner where the binding polypeptide has a sequence of between 6 and 30 amino acids in length, where the binding polypeptide sequence is composed by the steps of identifying the sequence of said binding partner; and, identifying 20% or more of the residues in said binding partner sequence; and where, for each of the identified residues within the binding partner sequence, selecting the residue at the corresponding position for inclusion in the sequence of the polypeptide sequence according to the corresponding residues given in Table 10.

### BRIEF DESCRIPTION OF THE DRAWINGS

FIGS. 1A-D. The complementary amino acid pairing (CAAP) boxes are located in the protein-protein interaction domains of exemplary well-known leucine-zipper proteins: FIG. 1A: human c-Jun/c-Fos heterodimer [PDB_1FOS] (SEQ ID NO: 274, SEQ ID NO: 275); FIG. 1B: Human Myc/Max heterodimer [PDB_1NKP] (SEQ ID NO: 276, SEQ ID NO: 277); FIG. 1C: Arabidopsis thaliana Hy5/Hy5 homodimer [PDB_20QQ] (SEQ ID NO: 278); and FIG. 1D: Yeast GCN4/GCN4 homodimer [PDB_2DGC] (SEQ ID NO: 279). (a) Alignment for the leucine-zipper (Leucine residues for the leucine zipper are shaded). (b) Alignment for the CAAP. The CAAP residues are underlined. The CAAP box is a cluster of the CAAP residues in the box.

FIGS. 2A-C. The CAAP boxes are also found in the protein-protein interaction domains of exemplary non-leucine-zipper proteins. FIG. 2A: S. aureus Ylan/Ylan homodimer [PDB_2ODM] (SEQ ID NO: 280); FIG. 2B: D. melanogaster DSX/DSX homodimer [PDB_1ZV1] (SEQ ID NO: 282, SEQ ID NO: 283, SEQ ID NO: 284); and FIG. 2C: Human PALS-1-L27N/Mouse PATJ-L27 hetero dimer [PDB_1VF6] (SEQ ID NO: 285); (a) protein sequence (SEQ ID NO: 286); (b) Alignment for the CAAP (SEQ ID NO: 287, SEQ ID NO: 288). The CAAP residues are underlined. The CAAP box is a cluster of the CAAP residues in the box.

FIG. 3. Frequency of each amino acid pairing in all the CAAP boxes found in the exemplary 77 crystal structure data.

FIGS. 4A-B. Composition (FIG. 4A) and pairing frequencies (FIG. 4B) of amino acids in the CAAP boxes from the exemplary 77 crystal structure data. The data from the parallel interactions and the antiparallel interactions are shown in dark bars and light bars, respectively. The bar graphs for cysteine, methionine, proline, and glutamine are not included since they are rarely appearing.

FIG. 5. Flowchart detailing one embodiment of the disclosed method.

FIGS. 6A-C. Diagrams of embodiments of three different CAAP oligopeptide types (Dark Arrows) to detect the target protein sequence (Light Arrows). FIG. 6A: monomer for parallel or antiparallel alignment; FIG. 6B dimer for antiparallel-linker-parallel or parallel-linker-antiparallel alignments; and FIG. 6C tetramer for antiparallel-linker-parallel-linker-antiparallel-linker-parallel or parallel-linker-antiparallel-linker-parallel-linker-antiparallel alignments.

FIGS. 7A-C. Exemplary dot blot analysis to detect the Cas9 target sequence using the His-tagged synthetic CAAP oligopeptides. FIG. 7A synthetic His-tagged CAAP oligopeptide monomer (PTD13 (SEQ ID NO: 28)); FIG. 7B synthetic His-tagged CAAP oligopeptide dimer (PTD14 (SEQ ID NO: 11)); and FIG. 7C no peptide (control). The densitometry plot profiles are shown under the blots. The CAAP interactions are shown in asterisks.

FIGS. 8A-B. Exemplary SDS-PAGE of the purified CAAP oligopeptide-AP fusion proteins: FIG. 8A: C9-813-92P (monomer, parallel), C9-813-93P (monomer, antiparallel), C9-813-CAA2 (dimer, parallel-linker-antiparallel); FIG. 8B: C9-813-CAA2 (dimer, parallel-linker-antiparallel), and C9-813-CAA4 (tetramer, parallel-linker-antiparallel-linker-parallel-linker-antiparallel).

FIGS. 9A-C. Exemplary dot blot analysis to detect the Cas9 target sequence using the recombinant CAAP oligopeptides-AP fusion proteins as 1st Ab: (FIG. 9A) C9-813-92P (monomer, parallel) (SEQ ID NO: 290); (FIG. 9B) C9-813-93P (monomer, antiparallel) (SEQ ID NO: 291, SEQ ID NO: 292); and (FIG. 9C) C9-813-CAA2 (dimer, parallel-linker-antiparallel) (SEQ ID NO: 293). The densitometry plot profiles are shown under the blots. The CAAP interactions are shown in asterisks.

FIG. 10A-B. Exemplary dot blot analysis to detect the Cas9 target sequence using the recombinant CAAP oligopeptides-AP fusion proteins as 1st Ab: (FIG. 10A) C9-813-CAA2 (dimer, parallel-linker-antiparallel) (SEQ ID NO: 293) and (FIG. 10B) C9-813 -CAA4 (tetramer, parallel-linker-antiparallel-linker-parallel-linker-antiparallel) (SEQ ID NO: 294). The densitometry plot profiles are shown under the blots.

FIGS. 11A-C. Exemplary dot blot (A) and western blot (C) analyses to detect the Cas9 proteins using the His-tagged synthetic CAAP oligopeptides. FIG. 11Aa and FIG. 11 Cb: synthetic His-tagged CAAP oligopeptide monomer (PTD13 (SEQ ID NO: 28)); FIG. 11Ab and FIG. 11Cc: synthetic His-tagged CAAP oligopeptide dimer (PTD14 (SEQ ID NO: 11)); and (Ac and Cd) no peptide (negative control). The Anti-Cas9 Ab-HRP conjugate was used as positive control to detect Cas9 protein (FIG. 11Ca). Two different forms of Cas9 proteins, Cas9 (no tag) and His-tagged Cas9, were spotted on NC membrane for dot blots, and resolved in 4-20% SDS-PAGE gel for Coomassie staining (FIG. 11B) or western blot analysis FIG. 11(C).

FIGS. 12A-E. Western blot analysis to detect binders for the synthetic CAAP oligopeptides in the whole proteome of E. coli BL21 Star DE3. The whole cell lysate of E. coli BL21 Star DE3 was resolved in 4-20% SDS-PAGE gel, and subjected to Coomassie staining (FIG. 12A) and western blot analysis using four different binding peptides: (FIG. 12B) synthetic His-tagged CAAP oligopeptide monomer (PTD13 (SEQ ID NO: 28)); (FIG. 12C) synthetic His-tagged CAAP oligopeptide dimer (PTD14 (SEQ ID NO: 11)); (FIG. 12D) synthetic linker-His-tag oligopeptide; and (FIG. 12E) no peptide (negative control).

FIGS. 13A-C. Dot blot analysis to detect the alkaline phosphatase target sequence using the synthetic His-tagged oligopeptides: (FIG. 13A) synthetic His-tagged CAAP oligopeptide monomer (PTD15 (SEQ ID NO: 295)); (FIG. 13B) synthetic His-tagged CAAP oligopeptide dimer (PTD16 (SEQ ID NO: 30)); and (FIG. 13C) synthetic linker-His-tag oligopeptide (control). The synthetic oligopeptide PTD7 (SEQ ID NO: 20) was used as an unrelated target (negative control). The CAAP interactions are shown in asterisks.

FIGS. 14A-C. Dot blot analysis to detect the PDGF-β target sequence (PTD10 (SEQ ID NO: 24)) using the synthetic His-tagged oligopeptides as 1st Ab: (FIG. 14A) synthetic His-tagged CAAP oligopeptide monomer (PTD17 (SEQ ID NO: 13)); (FIG. 14B) synthetic His-tagged CAAP oligopeptide dimer (PTD18 (SEQ ID NO: 31)); and (FIG. 14C) synthetic linker-His-tag oligopeptide (control). The synthetic oligopeptide PTD6 (SEQ ID NO: 19) was used unrelated target (negative control). The CAAP interactions are shown in asterisks.

FIGS. 15A-C. The synthetic CAAP oligopeptide (PTD14 (SEQ ID NO: 11)) directs significant induction of the non-specific Cas9-DNA interaction. (FIG. 15A) Schematic depiction for the cleavage of the human AAV1 region (510 bp) at the gRNA binding site as shown (SEQ ID NO: 296) by the RNA-guided Cas9 nuclease. (FIG. 15B) Effect of PTD14 (SEQ ID NO: 11) in different concentration of Cas9. The synthetic peptide PTD16 (SEQ ID NO: 30) was used as unrelated peptide control. (FIG. 15C) Effect of PTD14 (SEQ ID NO: 11) in presence or absence of gRNA.

FIGS. 16A-C. Dual detection using a purified polypeptide V5C2-L-HRPC2 with two CAAP box dimer arms designed to interact with V5 epitope and HRP. (FIG. 16A) Schematic depiction for the V5C2-L-HRPC2 with dual CAAP dimers to detect V5 epitope and HRP. (FIG. 16B) Amino acid sequence of the V5C2-L-HRPC2 (SEQ ID NO: 299) and the CAAP interaction with the target amino acid sequences (HRP_C1A, SEQ ID NO: 297; V5 epitope SEQ ID NO: 298). The CAAP interactions are shown in asterisks. (FIG. 16C) Dot blot analysis using synthetic polypeptides, PTD1 (SEQ ID NO: 14) (unrelated, control) and PTD19 (SEQ ID NO: 32) (part of V5 epitope), as target molecules in presence or absence of V5C2-L-HRPC2. The first interaction between V5 epitope and V5C2-L-HRPC2 was assessed by the second interaction between V5C2-L-HRPC2 and purified HRP protein. The first interaction was visualized using a HRP chromogenic substrate.

FIG. 17. Complementary amino acid pairing (CAAP) for 20 amino acids. The codon-complementary codon (c-codon) pairings for all possible CAAP interactions are shown top or bottom of the corresponding amino acid. Physicochemical properties of amino acids are shown in gray (hydrophobic), black (hydrophilic), white box (nonpolar/neutral), dotted box (polar/neutral), striped box (polar/negatively charged, acidic), and gray box (polar/positively charged, basic). Groups of CAAP interactions (↔) between two amino acids are shown: \textcircled{1} to \textcircled{9}, grouping by side chain hydrophobicity and polarity; asterisk(s), favorable amino acid pairings in the antiparallel alignment only (*) or both parallel/antiparallel alignments (**); and √, probable amino acid pairings consistent with the bonding rules. MW, molecular weight.

FIG. 18. The CCAAP boxes are found in the protein-protein interaction (PPI) site(s) of the leucine-zipper proteins. Global alignment and CAAP alignments in the linear representation of the four leucine-zipper proteins: Saccharomyces cerevisiae GCN4/GCN4 homodimer [PDB_2ZTA], Mus musculus NF-k-B essential modulator (NEMO) Homodimer [PDB_4OWF], Homo sapiens c-Jun/c-Fos heterodimer [PDB_1FOS], and Rattus norvegicus C/EBPA Homodimer [PDB_1NWQ]. Corresponding helical wheel representation is shown at the right-hand side of each CAAP alignment. In the linear representation, leucine residues for the leucine-zipper are indicated by Italic letters. The CAAP residues are highlighted with gray. The CCAAP boxes enclosing a cluster of the CAAP interactions are indicated by the gray boxes. The PPI sites are identified by a cluster of residues (asterisks) that have intermolecular interaction(s) in <3.6 Å distance, and indicated by gray bars on the top of the linear alignments. In the helical wheel representation, the new CAAP residues (that could not be identified in the linear representations) are underlined. Conversely, the CAAP residues (in the linear representations) losing the CAAP configuration in the helical wheel representation are indicated by dotted underline. The CAAP interactions in the helical wheel representation are indicated by gray lines. Hydrophobic and charged interactions are indicated by gray-dotted and gray-dashed lines, respectively. The possible CAAP interactions in the global alignments are indicated by letters (X, /, or \) between two molecules.

FIGS. 19A-B. The CCAAP boxes are found in the protein-protein interaction (PPI) site(s) of the non-leucine-zipper proteins. Global alignment and CAAP alignments in the linear representation of the five non-leucine-zipper proteins, three helix-helix (FIG. 19A) and two β-sheet-β-sheet (FIG. 19B) interactions: Saccharomyces cerevisiae Put3 Homodimer [PDB_1AJY], Salmonella enterica serovar Typhimurium TarH Homodimer [PDB_1VLT], Mus musculus E47-NeuroD1 Heterodimer [PDB_2QL2], Arenicola marina (lugworm) Arenicin-2 Homodimer [PDB_2L8X], and Laticauda semifasciata Erabutoxin Homodimer [PDB_1QKD]. Corresponding helical wheel representation is shown at the right-hand side of each CAAP alignment. In the linear representation, leucine residues for the leucine-zipper are indicated by Italic letters. The CAAP residues are highlighted with gray. The CCAAP boxes enclosing a cluster of the CAAP interactions are indicated by the gray boxes. The PPI sites are identified by a cluster of residues (asterisks) that have intermolecular interaction(s) in <3.6 Å distance, and indicated by gray bars on the top of the linear alignments. In the helical wheel representation, the new CAAP residues (that could not be identified in the linear representations) are underlined. Conversely, the CAAP residues (in the linear representations) losing the CAAP configuration in the helical wheel representation are indicated by dotted underline. The CAAP interactions in the helical wheel representation are indicated by gray lines. Hydrophobic and charged interactions are indicated by gray-dotted and gray-dashed lines, respectively. The possible CAAP interactions in the global alignments are indicated by letters (X or /) between two molecules. The PDB structure data also revealed some regional interactions that do not appear in the linear alignments: gray-arrow bars in PDB_1VLT and gray- and white-arrow bars in PDB_2QL2.

FIG. 20. The clustered appearance of the CAAP interactions in the PPI sites is statistically significant (♦♦♦♦♦, p<0.00001). Abundance of the CAAP interactions in the PPI and non-PPI sites was calculated by averaging % CAAP interactions from the CAAP alignment samples in FIGS. 18 and 19A-B (Table 9). The p value was obtained using a one-way ANOVA.

FIGS. 21A-D. CCAAP-based sAbs and rAbs can interact with the preselected peptide sequences of the target proteins. FIG. 21A: Dot blot analysis to detect the Cas9 target sequence using the His-tagged synthetic CCAAP oligopeptides (sAbs) as 1st Abs: synthetic His-tagged CCAAP sAb monomer (PTD13) and synthetic His-tagged CCAAP sAb dimer (PTD14). No peptide used for the negative control. CAAP interactions are shown in asterisks. FIG. 21B: Dot blot analysis to detect the Cas9 target sequence using the recombinant CCAAP oligopeptides-alkaline phosphatase (AP) fusion proteins (rAbs) as 1st Abs: C9-813-92P (monomer, parallel), C9-813-93P (monomer, antiparallel), and C9-813-CAA2 (dimer, parallel-linker-antiparallel). CAAP interactions are shown in asterisks. FIG. 21C: Dot blot and western blot analyses to detect the whole Cas9 proteins using the His-tagged CCAAP oligopeptide synthetic antibodies (sAbs). The CCAAP sAb monomer (PTD13) and dimer (PTD14) were used as 1st Abs. No 1st Ab was used for the negative control. The Anti-Cas9 Ab-HRP conjugate was used as positive control 1st Ab to detect Cas9 protein. The purified Cas9 protein (2 μg) was spotted on NC membrane for dot blots, and resolved in 4-20% SDS-PAGE gel for Coomassie staining or western blot analysis. FIG. 21D: Dot blot analysis to detect preselected target sequences in 7 additional target proteins using synthetic and recombinant antibodies (sAbs and rAbs). The rAbs are CCAAP oligopeptide Ab-AP fusion proteins. For the dot blots, the synthetic control peptide (5 μg) and target peptide (5 μg) were spotted on NC membrane. The dot blot images are original (uncropped) images from independent experiments. The dot blot images in the comparison group were obtained from the same experiment set. The blots in panels (a), (b), and (c) were incubated with the chromogenic substrates for 15 minutes to visualize the CCAAP sAb-Cas9 interaction. The dot blots in panel (d) were incubated with the chromogenic substrates for various lengths of incubation time (expose length) to obtain a sufficient intensity of the blot images. The Selected images are representing similar results from three independent experiments. The p values for the densitometry data were obtained using a one-way ANOVA.

### DETAILED DESCRIPTION

In one aspect, the present disclosure relates to methods for producing peptides, and especially peptides that can engage in interactions with other peptide sequences. In some embodiments, the present disclosure relates to the making of peptide-peptide or peptide-protein complexes, wherein a peptide is designed to interact with a known protein or a protein of known structure or sequence. In some aspects, the present disclosure relates to small peptides that are capable of interacting with other peptides or with proteins, said peptides being designed according to the methods and compositions described herein.

In some embodiments according to the methods and compositions disclosed herein, peptides can be designed to interact with one or more peptides or proteins of known structure or sequence by identifying the sequence of the target protein and, identifying the sequence of the binding peptide according to the following:

where the identified residue within the binding partner sequence is Phe, the residue at the corresponding position for inclusion in the binding peptide sequence is Lys or Glu; where the identified residue within the binding partner sequence is Leu, the residue at the corresponding position for inclusion in the binding peptide sequence is Gln, Lys, or Glu; where the identified residue within the binding partner sequence is Ser, the residue at the corresponding position for inclusion in the binding peptide sequence is Arg, Gly, Thr, or Ala; where the identified residue within the binding partner sequence is Thr, the residue at the corresponding position for inclusion in the binding peptide sequence is Ser, Gly, Cys, or Arg; where the identified residue within the binding partner sequence is Tyr, the residue at the corresponding position for inclusion in the binding peptide sequence is Ile or Val; where the identified residue within the binding partner sequence is Cys, the residue at the corresponding position for inclusion in the binding peptide sequence is Thr or Ala; where the identified residue within the binding partner sequence is Trp, the residue at the corresponding position for inclusion in the binding peptide sequence is Pro; where the identified residue within the binding partner sequence is Ile, the residue at the corresponding position for inclusion in the binding peptide sequence is Asn, Asp, or Tyr; where the identified residue within the binding partner sequence is Met, the residue at the corresponding position for inclusion in the binding peptide sequence is His; where the identified residue within the binding partner sequence is Asn, the residue at the corresponding position for inclusion in the binding peptide sequence is Ile or Val; where the identified residue within the binding partner sequence is Lys, the residue at the corresponding position for inclusion in the binding peptide sequence is Phe or Leu; where the identified residue within the binding partner sequence is Arg, the residue at the corresponding position for inclusion in the binding peptide sequence is Thr, Ala, Ser, or Pro; where the identified residue within the binding partner sequence is Pro, the residue at the corresponding position for inclusion in the binding peptide sequence is Arg, Gly, or Trp; where the identified residue within the binding partner sequence is His, the residue at the corresponding position for inclusion in the binding peptide sequence is Met or Val; where the identified residue within the binding partner sequence is Gln, the residue at the corresponding position for inclusion in the binding peptide sequence is Leu; where the identified residue within the binding partner sequence is Val, the residue at the corresponding position for inclusion in the binding peptide sequence is Asn, Asp, Tyr, or His; where the identified residue within the binding partner sequence is Ala, the residue at the corresponding position for inclusion in the binding peptide sequence is Ser, Gly, Cys, or Arg; where the identified residue within the binding partner sequence is Asp, the residue at the corresponding position for inclusion in the binding peptide sequence is Ile or Val; where the identified residue within the binding partner sequence is Glu, the residue at the corresponding position for inclusion in the binding peptide sequence is Phe or Leu; and where the identified residue within the binding partner sequence is Gly, the residue at the corresponding position for inclusion in the binding peptide sequence is Thr, Ala, Ser, or Pro. In some embodiments, not all of the residues of the binding peptide will be determined according to the relationships disclosed herein. In some embodiments, for example, every other residue, every third residue, or two of every three residues will be determined according to the disclosed relationships.

“Subject” as used herein, has its customary and ordinary meaning as understood by one of skill in the art in view of this disclosure. It refers to a human or a non-human animal, for example selected or identified for a diagnosis, treatment, inhibition, amelioration of a disease, disorder, condition, or symptom. “Subject suspected of having” has its customary and ordinary meaning as understood by one of skill in the art in view of this disclosure. It refers to a subject exhibiting one or more indicators of a disease or condition. In certain embodiments, the disease or condition may comprise one or more of a disease, disorder, condition, or symptom.

“Administering” has its customary and ordinary meaning as understood by one of skill in the art in view of this disclosure. It refers to providing a substance, for example a pharmaceutical agent, dietary supplement, or composition, to a subject, and includes, but is not limited to, administering by a medical professional and self-administration. Administration of the compounds disclosed herein or the pharmaceutically acceptable salts thereof can be via any of the accepted modes of administration for agents that serve similar utilities such as are consistent with the formulation of said compounds. Oral administrations are customary in administering the compositions that are the subject of the preferred embodiments. In some embodiments, administration of the compounds may occur outside the body, for example, by apheresis or dialysis.

In some embodiments, the methods of the present disclosure contemplate the administration of one or more compositions useful for the amelioration or treatment of one or more disorders, diseases, conditions, or symptoms.

Standard pharmaceutical and/or dietary supplement formulation techniques are used, such as those disclosed in Remington's The Science and Practice of Pharmacy, 21st Ed., Lippincott Williams & Wilkins (2005), incorporated herein by reference in its entirety. Accordingly, some embodiments include pharmaceutical and/or dietary supplement compositions comprising, consisting of, or consisting essentially of: (a) a safe and therapeutically effective amount of one or more compounds described herein, or pharmaceutically acceptable salts thereof; and (b) a pharmaceutically acceptable carrier, diluent, excipient or combination thereof.

The term “pharmaceutically acceptable carrier” or “pharmaceutically acceptable excipient” has its customary and ordinary meaning as understood by one of skill in the art in view of this disclosure. It includes any and all appropriate solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations of the compounds disclosed herein. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

The choice of a pharmaceutically-acceptable carrier to be used in conjunction with the one or more compounds for administration as described herein can be determined by the way the compound is to be administered.

In some embodiments, the methods of the present disclosure contemplate topical or localized administration. In some embodiments, the methods of the present disclosure contemplate systemically or parenterally, such as subcutaneously, intraperitoneally, intravenously, intraarterially, orally, enterically, subdermally, transdermally, sublingually, transbuccally, rectally, or vaginally.

The present disclosure describes binding peptides that interact with proteins or peptides of known structure or sequence. In certain embodiments according to the methods and compositions disclosed herein, said binding peptides may comprise, consist of, or consist essentially of, one or more sequences determined by the steps of: identifying the sequence of the target protein or peptide; and for each residue of the target protein or polypeptide, placing a corresponding residue in the sequence of the binding peptide according to the following relationships: where the identified residue within the binding partner sequence is Phe, the residue at the corresponding position for inclusion in the binding peptide sequence is Lys or Glu; where the identified residue within the binding partner sequence is Leu, the residue at the corresponding position for inclusion in the binding peptide sequence is Gln, Lys, or Glu; where the identified residue within the binding partner sequence is Ser, the residue at the corresponding position for inclusion in the binding peptide sequence is Arg, Gly, Thr, or Ala; where the identified residue within the binding partner sequence is Thr, the residue at the corresponding position for inclusion in the binding peptide sequence is Ser, Gly, Cys, or Arg; where the identified residue within the binding partner sequence is Tyr, the residue at the corresponding position for inclusion in the binding peptide sequence is Ile or Val; where the identified residue within the binding partner sequence is Cys, the residue at the corresponding position for inclusion in the binding peptide sequence is Thr or Ala; where the identified residue within the binding partner sequence is Trp, the residue at the corresponding position for inclusion in the binding peptide sequence is Pro; where the identified residue within the binding partner sequence is Ile, the residue at the corresponding position for inclusion in the binding peptide sequence is Asn, Asp, or Tyr; where the identified residue within the binding partner sequence is Met, the residue at the corresponding position for inclusion in the binding peptide sequence is His; where the identified residue within the binding partner sequence is Asn, the residue at the corresponding position for inclusion in the binding peptide sequence is Ile or Val; where the identified residue within the binding partner sequence is Lys, the residue at the corresponding position for inclusion in the binding peptide sequence is Phe or Leu; where the identified residue within the binding partner sequence is Arg, the residue at the corresponding position for inclusion in the binding peptide sequence is Thr, Ala, Ser, or Pro; where the identified residue within the binding partner sequence is Pro, the residue at the corresponding position for inclusion in the binding peptide sequence is Arg, Gly, or Trp; where the identified residue within the binding partner sequence is His, the residue at the corresponding position for inclusion in the binding peptide sequence is Met or Val; where the identified residue within the binding partner sequence is Gln, the residue at the corresponding position for inclusion in the binding peptide sequence is Leu; where the identified residue within the binding partner sequence is Val, the residue at the corresponding position for inclusion in the binding peptide sequence is Asn, Asp, Tyr, or His; where the identified residue within the binding partner sequence is Ala, the residue at the corresponding position for inclusion in the binding peptide sequence is Ser, Gly, Cys, or Arg; where the identified residue within the binding partner sequence is Asp, the residue at the corresponding position for inclusion in the binding peptide sequence is Ile or Val; where the identified residue within the binding partner sequence is Glu, the residue at the corresponding position for inclusion in the binding peptide sequence is Phe or Leu; and where the identified residue within the binding partner sequence is Gly, the residue at the corresponding position for inclusion in the binding peptide sequence is Thr, Ala, Ser, or Pro.

In certain embodiments according to the methods and compositions disclosed herein, said binding peptide sequence may be designed to be parallel to the direction of the target sequence (i.e., with the identified residues in the binding peptide sequence placed from N terminal to C-terminal, corresponding to the residues of the target peptide in their N-terminal to C-terminal orientation) or may be designed to be antiparallel to the direction of the target sequence (i.e., with the identified residues in the binding peptide sequence placed from N terminal to C-terminal, corresponding to the residues of the target peptide in their C-terminal to N-terminal orientation). In some embodiments, a portion, but not all, of the residues of the binding peptide will be determined according to the disclosed relationships. In some embodiments, for example, every other residue, every third residue, one of every three residues, two of every three residues, or one, two, or three out of every four residues will be determined according to the disclosed relationships. In some embodiments, the residues to be determined according to the disclosed relationships will follow a pattern such as [OOXOOOXOO]ₙ, [OOOXOXOOO]ₙ, and [OOOOOXOOOO]ₙ (Where “O” represents a residue determined according to the disclosed relationships, “X” represents any residue, and n represents any integer). In some embodiments, the residues to be determined according to the disclosed relationships will follow a pattern such as [OOO′OOOO′OO]ₙ, [OOOO′OO′OOO]ₙ, and [OOOOOO′OOOO]ₙ (Where “O” represents a residue determined according to the disclosed relationships with respect to a first target protein or peptide, and “O′” a residue determined according to the disclosed relationships with respect to a second target protein or peptide, and n represents any integer).

In some embodiments, without respect to their specific placement within the sequence of the binding peptide, all of the residues of the binding peptide will be selected according to the relationships given herein. In some embodiments, without respect to their specific placement within the sequence of the binding peptide, less than all of the residues of the binding peptide will be selected according to the relationships given herein. In some embodiments, without respect to their specific placement within the sequence of the binding peptide, the percentage of residues within the binding peptide sequence that are selected according to the relationships given herein is 10-30%. In some embodiments, without respect to their specific placement within the sequence of the binding peptide, the percentage of residues within the binding peptide sequence that are selected according to the relationships given herein is between 20-40%, 30-50%, 40-60%, 50-70%, 60-80%, 70-90%, 20-90%, 30-90%, or 30-80%. In some embodiments, without respect to their specific placement within the sequence of the binding peptide, the percentage of residues within the binding peptide sequence that are selected according to the relationships given herein is greater than 90%. In some embodiments, without respect to their specific placement within the sequence of the binding peptide, the percentage of residues within the binding peptide sequence that are selected according to the relationships given herein is, or is at least, 20%, 30%, 40%, 50%, 60%, 70%, 80%, 90%, 95%, or 100%, or a range selected from any two of the preceding values.

In some embodiments according to the methods and compositions described herein, a library of binding peptides may be developed according to the relationships and criteria described herein. Said libraries may be screened, such as by surface plasmon resonance spectroscopy, nuclear magnetic resonance spectroscopy, fluorescence resonance energy transfer, fluorescence quenching, Raman spectroscopy, ELISA, western blotting, or dot blot or other methods as are known to those of skill in the art, for binding to the selected target sequence or protein. Sequences identified as having desirable binding properties or other desirable properties may optionally be subjected to another round of design, such as by placing alternate residues still in compliance with the relationships described herein for the design of binding peptides, or by altering the location or register of one or more of the residues selected according to the criteria described herein. Additional rounds of screening and optimization may follow.

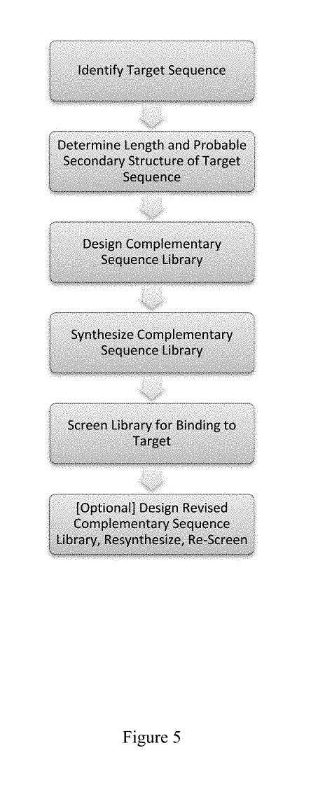

In some embodiments, the method is structured according to the steps shown in FIG. 5. In the first box, a target sequence is identified, and may comprise any segment of the sequence of a target protein or peptide. Exemplary target sequences may be between 2 and 100 amino acids, 2 and 50 amino acids, between 2 and 25 amino acids, between 5 and 20 amino acids, or between 5 and 15 amino acids in length. Optionally, said target sequence may be identified based on examination of the three-dimensional structure of the target protein or peptide. Optionally, said target sequence may be identified based on sequence analysis, sequence alignment, or structure prediction based on the sequence of the target protein or peptide.

The next box illustrates an additional step according to some embodiments of the present method, wherein the length and probable secondary structure of the target sequence can be determined. This may be done according to such criteria as are suitable for the target protein, such as by observing the boundaries of secondary structure elements (e.g. Beta strands, alpha helices, loops, knots, pseudoknots, beta hairpins, 310 helices, and the like) within the three dimensional structure of the target protein or peptide, or by predicting the secondary structures within the target protein using sequence alignments or sequence analysis tools such as are known in the art. Target sequences may be of any length appropriate for the interaction of the binding peptide with the target protein, and as noted herein, exemplary target sequences may be between 2 and 100 amino acids, 2 and 50 amino acids, between 2 and 25 amino acids, between 5 and 20 amino acids, or between 5 and 15 amino acids in length.

The third box depicts a step according to some embodiments of the present method, wherein a binding peptide is designed according to the relationships and design criteria described herein. For example, where the target sequence is primarily alpha helical, CAAP residues corresponding to the residues of the target sequence according to the relationships disclosed herein may be placed at one or two of every three positions within the designed sequence, or when the target sequence comprises significant beta strand character, CAAP residues corresponding to the residues of the target sequence according to the relationships disclosed herein may be placed at every other position within the designed sequence. Likewise, one of skill in the art may determine proper placement of CAAP residues in order to interact with other secondary structure elements, including but not limited to loops, knots, pseudoknots, beta-hairpins, and 3₁₀ helices. In some embodiments, the size of the binding peptide may be commensurate with the size of the target sequence, and exemplary binding peptide sequences may be between 2 and 100 amino acids, 2 and 50 amino acids, between 2 and 25 amino acids, between 5 and 20 amino acids, or between 5 and 15 amino acids in length. The contemplated size of the binding peptide, or the binding portion of a protein, is, is about, is at least, or is not more than, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, or 100 amino acids long, or a range defined by any two of the preceding values.

Optionally, multiple binding sequences may be designed, for example incorporating alternate CAAP residues as disclosed herein and shown in Table 1 or having a different number or placement of the CAAP residues. Exemplary libraries may comprise more than one peptide sequences, between 1 and 5 peptide sequences, between 2 and 10 peptide sequences, 12 or fewer peptide sequences, 24 or fewer peptide sequences, 48 or fewer peptide sequences, 96 or fewer peptide sequences, 192 or fewer peptide sequences, 384 or fewer peptide sequences, 1536 or fewer peptide sequences, or greater than 1536 peptide sequences, or a range between any of the preceding values. Such a library has considerable advantages over conventional library screening methods. For example, while a fully random library of 10-mer peptides would comprise 10¹³ peptides, an amount which could not reasonably be screened with specificity, by applying the methods described herein, library size and complexity can be reduced by 10⁹-10¹⁰-fold, reducing the size of the library to one in which each peptide can reasonably be individually screened.

The next box depicts a step according to some embodiments of the present method, wherein a library of designed binding sequences is synthesized or produced, for example by heterologous gene expression. In some embodiments, DNA sequences corresponding to the sequences of the designed binding peptides can be obtained and transformed into appropriate organisms for expression using such methods as are known in the art (see, for example, Green, M. R. and Sambrook, J., Molecular Cloning: A Laboratory Manual, 4^{th} ed. Volume 3, Cold Spring Harbor Laboratory Press (2012); and Greenfield, E.A., ed., which is hereby incorporated by reference for purposes of its description of genetic modification of organisms and heterologuous protein production). Purification of expressed peptides may be carried out by such methods as are known in the art and may optionally include high performance liquid chromatography, precipitation, and/or affinity purification such as, for example, metal affinity purification, glutathione-S-transferase affinity purification, protein A affinity purification, or Ig-Fc affinity purification. Binding peptides may be synthesized using for example solid phase or liquid phase methods, for example, those described in Jensen, K. J. et al., eds. Peptide Synthesis and Applications, 2n^{d} ed., Humana Press (2013), which is hereby incorporated by reference with respect to its disclosure of methods for the synthesis, purification, and characterization of peptides.

The next box in the figure depicts a step according to some embodiments of the present method, wherein and as noted herein, binding peptide libraries are screened for binding to the target protein using such methods as or known in the art and/or are described herein.

The final box depicts a step wherein optionally, sequences screened may be revised, for example by designing new peptides retaining residues shown to be important to binding, and by varying the position and or composition of the remaining CAAP residues utilizing the relationships disclosed herein and in Table 4. A redesigned library may then be produced or synthesized, and screened, as described, in order to identify peptides with optimal binding activity.

In some embodiments, the binding peptide may comprise one part of a larger fusion peptide. Such a fusion polypeptide may comprise, for example, one or more binding peptides and optionally, an effector peptide. In some embodiments, an effector peptide may comprise a therapeutic or diagnostic peptide, an affinity tag, an antibody, a signaling protein, an enzyme, an inhibitor, or any such peptide moiety as may be desired to be bound to the target protein via the binding peptide. In some embodiments, a fusion peptide comprises a linker as described herein or as known to one of skill in the art. In some embodiments, the binding peptide may comprise the full length of a given fusion polypeptide sequence. In some embodiments, the binding peptide may comprise less than the full length of a given fusion polypeptide sequence. In some embodiments, the binding peptide may comprise between 10% and 100% of the length of a given fusion polypeptide sequence. In some embodiments the binding peptide may comprise between 20% and 90% of the length of a given fusion polypeptide sequence. In some embodiments, the binding peptide may comprise less than 90%, less than 80%, less than 70%, less than 60%, less than 50%, less than 40%, less than 30%, less than 20%, less than 10%, or less than 5% of the length of a given fusion polypeptide sequence. In some embodiments, a fusion polypeptide may comprise one, two, three, four, or more than four binding peptides. In some embodiments, a fusion polypeptide may be from 10 to 600 amino acids in length. In some embodiments, a fusion polypeptide may be from 10 to 500 amino acids in length. In some embodiments, a fusion polypeptide may be from 20 to 400 amino acids, from 30 to 300 amino acids, from 40 to 200 amino acids, from 50 to 100 amino acids, from 10 to 100 amino acids, from 20 to 100 amino acids, from 10 to 200 amino acids, or from 20 to 200 amino acids in length, or a range defined by any two of the preceding values (e.g. 20 to 600 amino acids).

In some embodiments, the binding peptide may be linked to, or may comprise, an affinity tag or an enzyme. Exemplary tags or enzymes include but are not limited to metal affinity tags such as His₆, glutathione-S-transferase, protein A, lectins, immunoglobulin constant regions, fluorescent proteins such as the Green Fluorescent Protein and the like, and/or horseradish peroxidase.

In some embodiments, a sequence may be designed to bind to multiple targets. For example, a sequence may have 50% of its residues selected according to the relationships described herein with respect to the sequence of one target sequence, and 50% of its residues selected according to the relationships described herein with respect to the sequence of a second binding target. The second binding target may be a second target protein or may be a second sequence within a single target protein. The division of residues may be more or less than 50%-50%, for example, from 70-90% to from 10-30%, from 60-80% to from 20-40%, from 50-70% to from 30-50%, from 40-60% to from 40-60%, from 30-50% to from 50-70%, from 20-40% to from 60-80%, or from 10-30% to from 70-90%. Likewise, in some embodiments a sequence may be designed to bind to three or more sequences by allocating a percentage of the residues in the binding peptide sequence to interact according to the relationships described herein with the sequences of three or more target sequences.

In certain embodiments, said binding peptides may exist in single copies. In certain other embodiments, said binding peptides may be fused to other binding peptides. In some embodiments, said binding peptides may be present as dimers, trimers, tetramer, pentamers, hexamers, or the like. In some embodiments, said binding peptides may be fused to identical binding peptides. In some embodiments, two or more different binding peptides may be fused together. In some embodiments said binding peptides may be fused in the same orientation (i.e., C terminus to N terminus). In some embodiments, said peptides may be fused in the opposite orientation (i.e., N terminus to N terminus, or C terminus to C terminus). In some embodiments, said binding peptides may be linked together by a peptide linker. In some embodiments, said peptide linker may comprise, consist of, or consist essentially of, one or more sequences such as (G)ₙ (SEQ ID NO: 2), (GS)ₙ (SEQ ID NO: 3), (GGSGG)ₙ (SEQ ID NO: 4), (GGGS)ₙ (SEQ ID NO: 5), CYPEN (SEQ ID NO: 6), or KTGEVNN (SEQ ID NO: 7) or the like. In some embodiments, said binding peptides may be linked together by a nonpeptide linker. Exemplary nonpeptide linkers include but are not limited to polyethylene glycol, polypropylene glycol, polyols, polysaccharides or hydrocarbons. In some embodiments, each binding peptide within the fusion binds to the same target. In some embodiments, the binding peptides within the fusion bind to different targets.

In some embodiments, the present disclosure describes peptides that interact with target proteins. In some embodiments, said target proteins may comprise, consist of, or consist essentially of, one or more of human c-Jun/c-Fos heterodimer; Human Myc/Max heterodimer; Arabidopsis thaliana Hy5/Hy5 homodimer; Yeast GCN4/GCN4 homodimer; Ylan/Ylan homodimer; Drosophila melanogaster DSX/DSX homodimer; human PALS-1-L27N/Mouse PATJ-L27 heterodimer; Staphylococcus pyogenes Cas9; Escherichia coli alkaline phosphatase (AP); and Human Platelet-Derived Growth Factor (PDGF)/PDGF Receptor (PDGFR) complex. In some embodiments, the binding peptides comprise, consist of, or consist essentially of, one or more of the sequences ELDKAGFIKRQL (SEQ ID NO: 14), LEERGVKDRQLQ (SEQ ID NO: 15), LEILRAKDLALE (SEQ ID NO: 16), LEQIKIRLF (SEQ ID NO: 17), LSGLNEQRTQ (SEQ ID NO: 18), YDVDAIVPQC (SEQ ID NO: 19), CLTYDSHYLQ (SEQ ID NO: 20), LVAHVTSRKC (SEQ ID NO: 21), EYRLYLRALC (SEQ ID NO: 22), IEIVRKKPIF (SEQ ID NO: 23), IEIVRKKPIFC (SEQ ID NO: 24), CEDRLQSYDLD (SEQ ID NO: 25), EKLYLYYLQ (SEQ ID NO: 26), EKLYLYYLQC (SEQ ID NO: 27), LEQIKIRLFGSGSHHHHHH (SEQ ID NO: 28), LLQVDVILLCYPENLEQIKIRLFGSGSHHHHHH (SEQ ID NO: 11), LSRAYLSYEGSGSHHHHHH (SEQ ID NO: 29), EYRLYLRALCYPENLSRAYLSYEGSGSHHHHHH (SEQ ID NO: 30), EDRLQSYDLDGSGSHHHHHH (SEQ ID NO: 13), DLDYAQLRDKCYPENEDRLQSYDLDGSGSHHHHHH (SEQ ID NO: 31), GKPIPNPLLGLDST (SEQ ID NO: 32), ELDKAGFIKRQLC (SEQ ID NO: 33), LLQVDVILLHHHHHHLEQIKIRLF (SEQ ID NO: 34), and/or CFFDSLVKQ (SEQ ID NO: 35), or any combination or derivative thereof.

In some embodiments, binding peptides according to the methods and compositions as disclosed herein may be conjugated to a therapeutic moiety. Exemplary therapeutic moieties include but are not limited to, antibacterial agents, antifungal agents, chemotherapeutic agents, and biologics. In some embodiments the binding peptides according to the methods and compositions disclosed herein may be conjugated to a detectable moiety, including, for example, a fluorescent label, a radiolabel, an enzyme, a colorimetric label, a spin label, a metal ion binding moiety, a nucleic acid, a polysaccharide, or a polypeptide. In some embodiments, binding peptides as disclosed herein or made according to the methods described herein bind to or interact with biomarkers of human or animal diseases, disorders, conditions, or symptoms. It is contemplated that such peptides could be attached to a detectable moiety as described herein to provide for diagnosis, prognosis, or identification of said human or animal diseases, disorders, conditions, or symptoms.

Also contemplated herein are methods of treating diseases or disorders in a subject by administering the peptides as disclosed herein, including administering peptides designed and/or made according to the methods described herein, to a subject in need thereof. The present disclosure contemplates the making of peptide-protein complexes wherein said complex may occur in vivo or wherein said complexes are made by contacting the binding peptides disclosed herein or made by the methods as disclosed herein with a target protein or peptide, and wherein said contacting occurs in vivo. The making of said complexes or the contacting of said binding peptides with said target protein or peptide in vitro or ex vivo is also contemplated. Some embodiments according to the methods and compositions of the present disclosure provide for a composition comprising, consisting of, or consisting essentially of, one or more of the binding peptides as disclosed herein or made according to the methods disclosed herein, and optionally one or more excipients as described herein. Said composition may be prepared according to methods known in the art for delivery to the body of a subject, for example by parenteral, topical, subcutaneous, intramuscular, intraocular, intracerebral, intravenous, intraarterial, oral, ocular, intranasal, or transdermal delivery.

Specific targeting of a protein area by pre-selected sequence would be extremely useful for many branches of biotechnological sciences including medical diagnostics, disease prevention/eradication, biomedical engineering, and metabolic engineering. Antibodies are the present workhorse for detecting target proteins because they recognize epitopes with high affinity and specificity. Currently, however, production of antibodies for the pre-selected target sequence is tedious, time-consuming, and expensive. In addition, it is difficult to produce antibodies in very large quantities. As a large protein with disulfide bonds, moreover, antibodies are relatively fragile and unsuitable for certain applications such as delivery into live cells and very small biological environments. Therefore, it is an important goal to develop small biopolymers that retain the favorable molecular recognition characteristics of antibodies but that can be easily synthesized in large amounts. In the present study, we provide a new concept for the protein detection that has a potential to at least in part replace antibodies for protein targeting. Certain embodiments of the methods and compositions described herein are illustrated by the following non-limiting examples.

### Example 1

### Development of the Design Principles

We summarized pairings of amino acids in Table 1. This pairing is named “complementary amino acid pairing (CAAP)”. Using the hydrophobicity grouping of amino acids [Kyte J, and Doolittle RF (1982) J Mol Biol 157: 105-132], we found that there are four different types of pairing relationships between the CAAP residues: hydrophilic-hydrophobic (44%), hydrophilic-neutral (20%), neutral-hydrophobic (13%), and neutral-neutral (23%). There are no hydrophilic-hydrophilic and hydrophobic-hydrophobic relationships. Interestingly, 38% of the CAAP interactions (shaded in Table 1) belong to the acceptable amino acid pairings [Root-Bernstein, R. S. J Theor Biol. 1982 Feb. 21; 94(4):885-94]. In addition, the most CAAP interactions have a good stereochemical arrangement: the high molecular weight (bulky) side chains are pairing with the low molecular weight (small) side chains, and vice versa. These observations led us to postulate that the physicochemical and stereochemical natures of the CAAP relationships between two polypeptide chains may provide an attractive environment for protein-protein interaction.

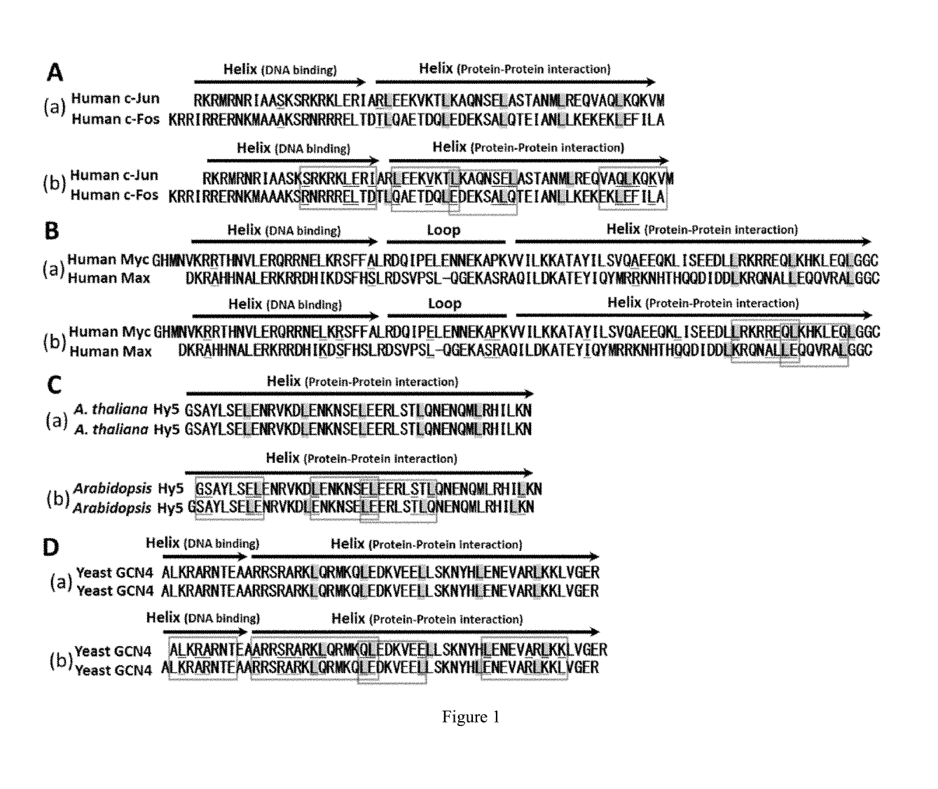

We first focused on finding the CAAP interactions in the protein-protein interaction structure database from the protein data bank (PDB). We first examined the well-known leucine zipper proteins: human c-Jun/c-Fos heterodimer [PDB_1FOS]; Human Myc/Max heterodimer [PDB_1NKP]; Arabidopsis thaliana Hy5/Hy5 homodimer [PDB_20QQ]; and Yeast GCN4/GCN4 homodimer [PDB_2DGC]. As shown in FIG. 1A-D, we do not see CAAP residues in the leucine-zipper alignment. However, many CAAP interactions are revealed in the alignment with one amino acid shift. Remarkably, 80% (52 out of 65 pairings) of the CAAP residues are clustered in the protein-protein interaction domains. Clusters of CAAP residues are indicated by the box called “CAAP box”. The cut-off criteria for a CAAP box was at least 8 or more amino acid pairings and 37.5% or more of them must be CAAPs. We found 11 CAAP boxes in the protein-protein interaction domains and 2 CAAP boxes in the DNA binding domains (FIG. 1Ab-1Bb-1Cb-1Db). Interestingly, 90% of leucine residues for the leucine zippers are linked with the CAAP interactions (FIG. 1Ab-1Bb-1Cb-1Db). In fact, 60% of leucine residues for the leucine zippers directly contributed to the CAAP interactions (FIG. 1Ab-1Bb-1Cb-1Db). These features could be an additional explanation of how the leucine zipper form a strong α-helical dimer.

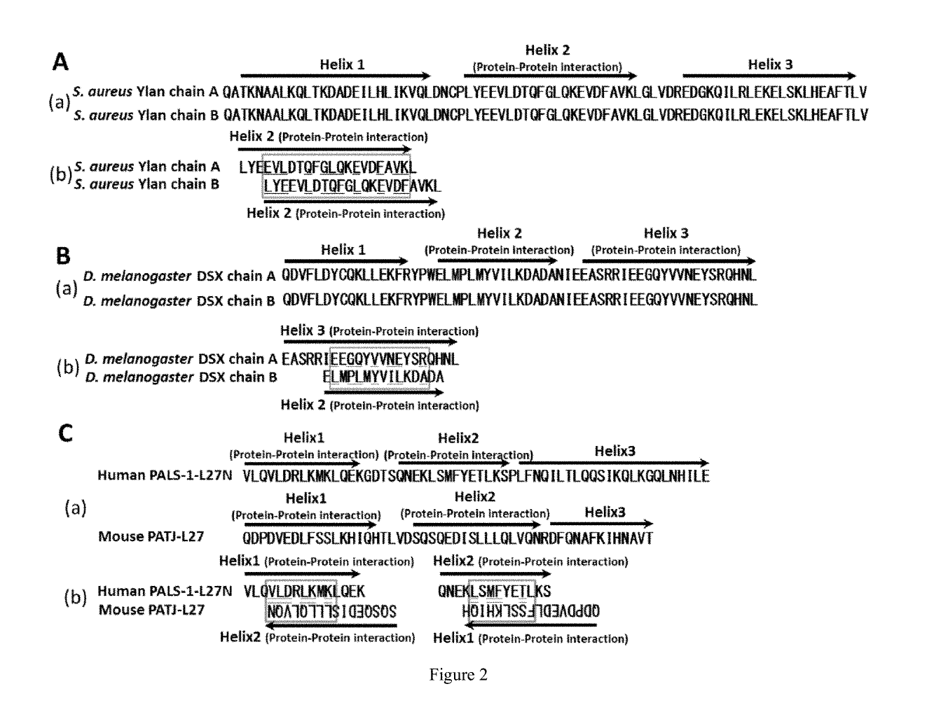

Next, we expanded the search for the CAAP boxes into some non-leucine-zipper proteins: Staphylococcus aureus Ylan/Ylan homodimer [PDB_20DM]; Drosophila melanogaster DSX/DSX homodimer [PDB_1ZV1]; and human PALS-1-L27N/Mouse PATJ-L27 hetero dimer [PDB_1VF6]. The CAAP boxes are also found in all protein-protein interaction domains of the non-leucine-zipper proteins (FIG. 2Ab-2Bb-2Cb). We have examined a total 77 protein structures (See Table 4) which were selected for their relatively simple protein-protein interaction structure and clear alignment of side chains in order to limit the involvement of any potential parameters. We found CAAP boxes in all protein-protein interaction domains in 76 of the 77 proteins examined. The only exception was the homodimer of Pseudopleuronectes americanus Type I antifreeze protein [PDB_4KE2]. This protein has a very unusual polypeptide sequence [121 (62%) alanine residues in total 196 amino acids], thus no CAAP box is found in the homodimer structural alignment. We found 63 CAAP boxes in parallel alignments and 43 CAAP boxes in antiparallel alignments in the protein-protein interaction domains of the 83 protein structures.

### Designing Polypeptide Sequence to Target Pre-Selected Polypeptide Sequence

We assessed the composition of all amino acid pairings in the CAAP boxes to obtain information on pairing preference and how the CAAPs were spaced out. First, we wrote a simple computational program to count all amino acid pairings in two different sets, parallel alignment and antiparallel alignment.

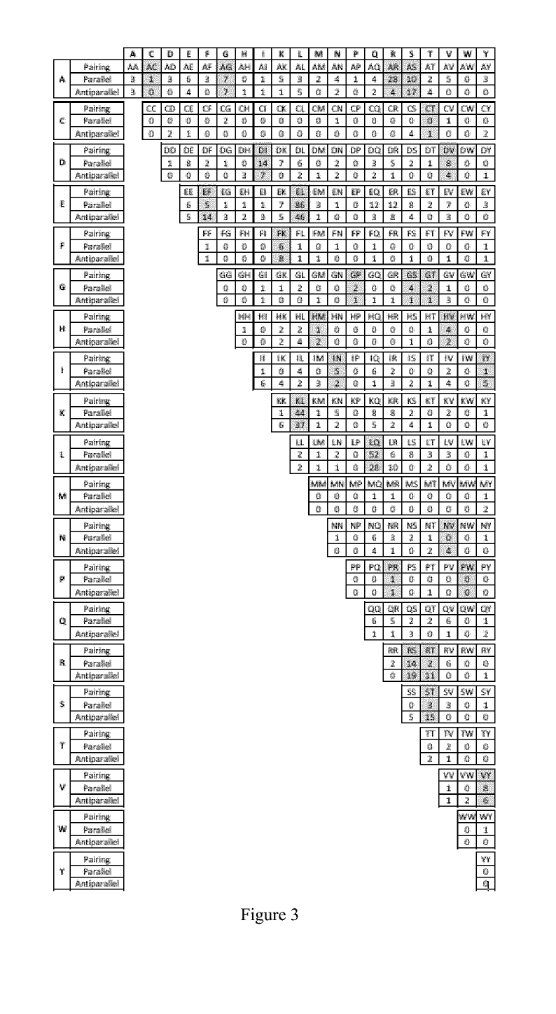

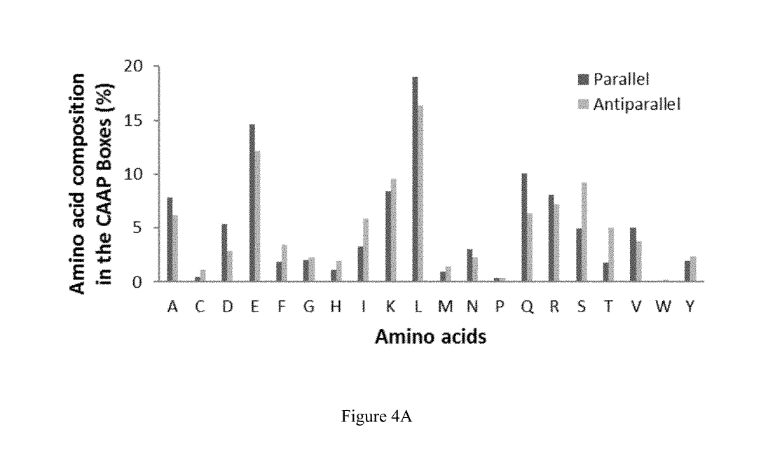

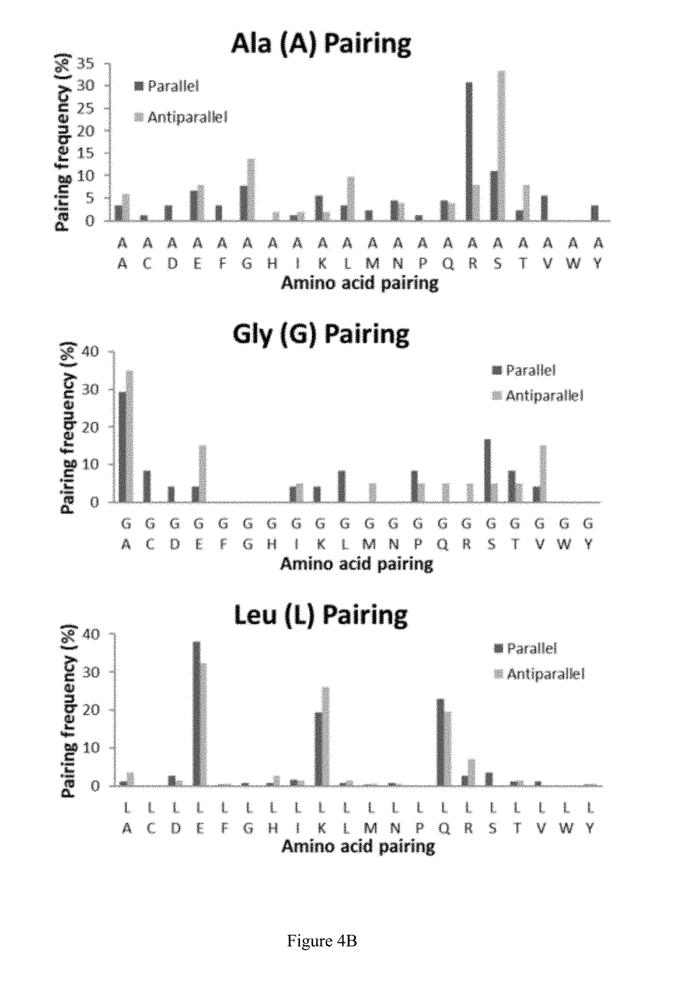

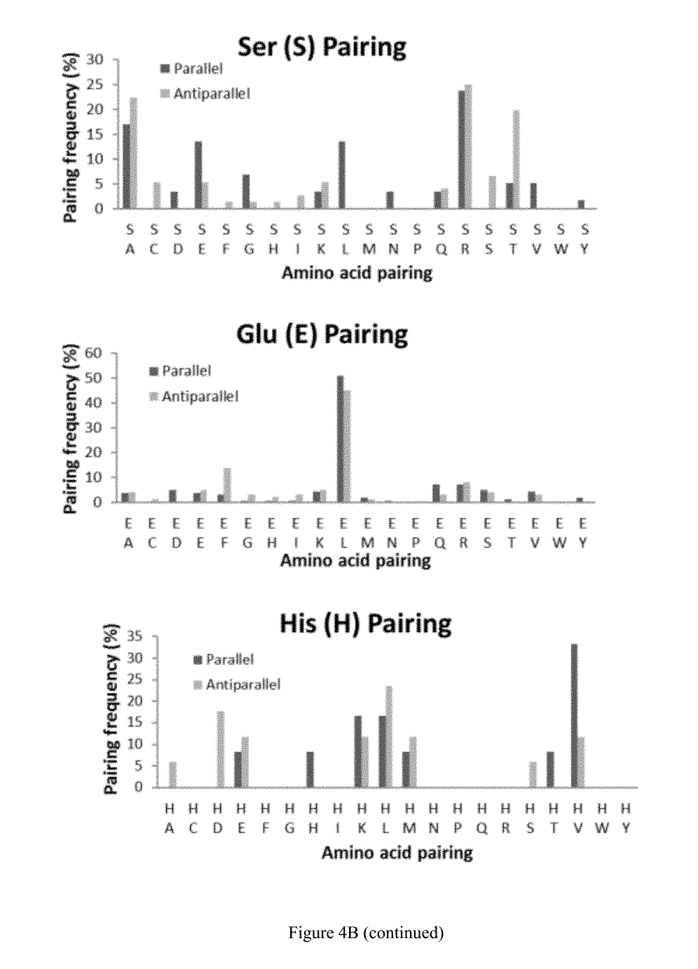

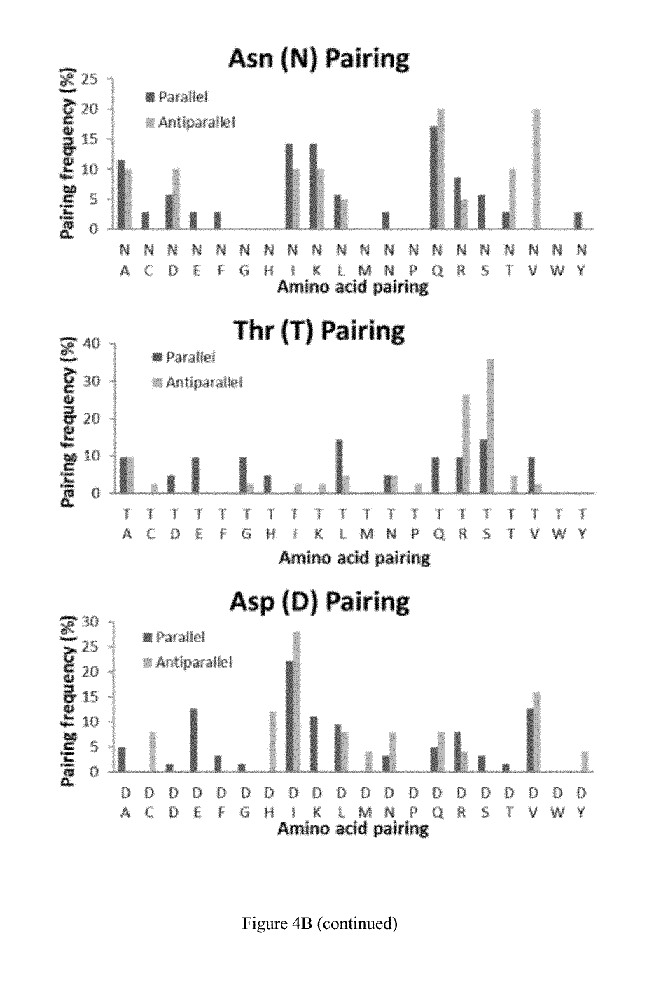

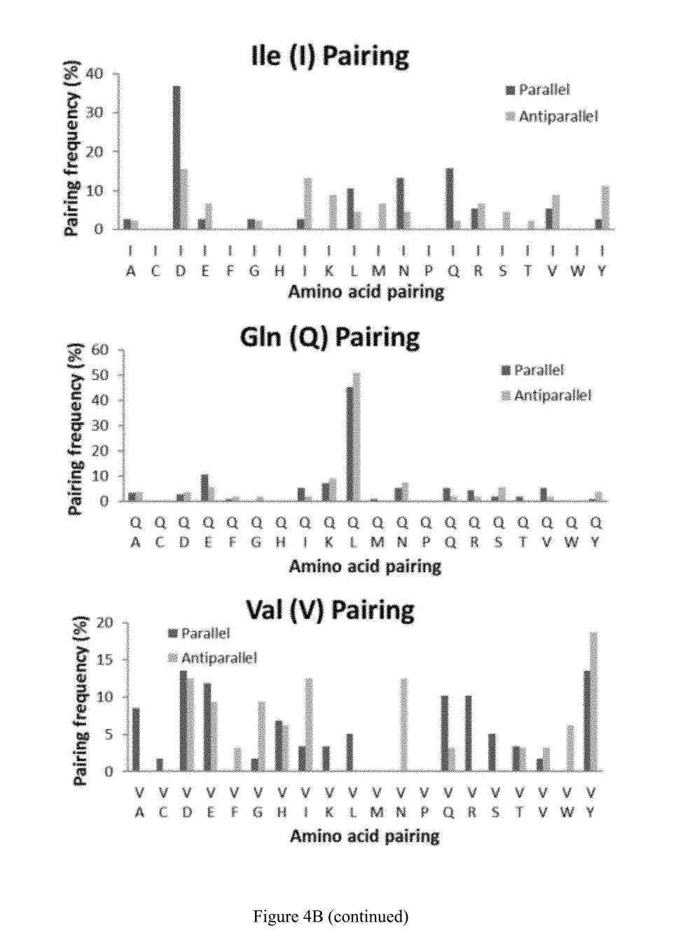

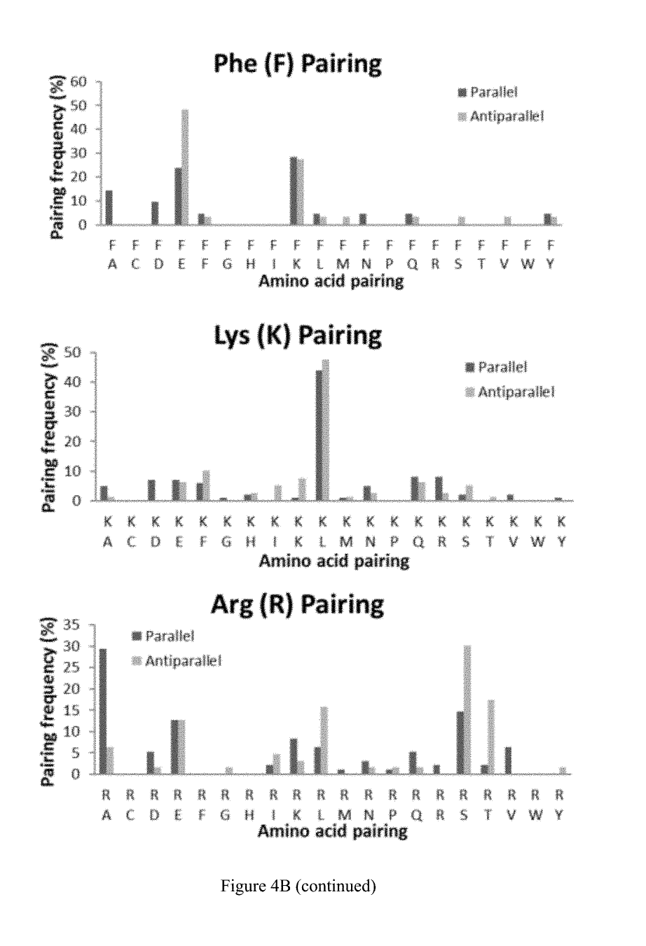

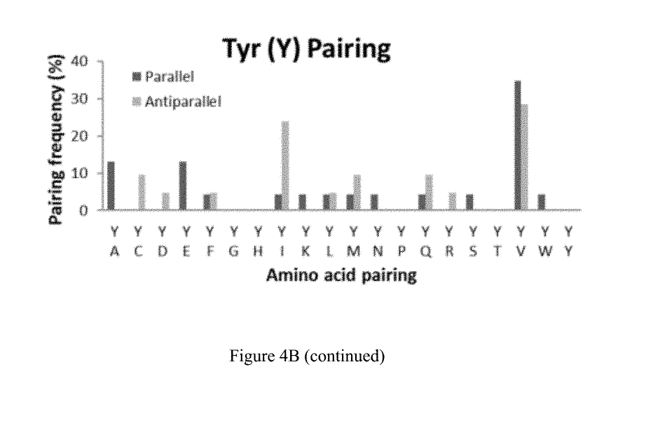

The numbers are shown in FIG. 3 and FIG. 4. This data was then used for designing oligopeptide sequences to target a pre-selected polypeptide sequence from a oligopeptide or protein. In a window with 9 or 10 pairings, we tried to mimic the natural spacing examples observed from the collected data: OOXOOOXOO, OOOXOXOOO, and OOOOOXOOOO [where O is CAAP interaction and X is non-CAAP interaction]. For each designated CAAP or non-CAAP, in general, we selected the most frequent pairing partner according to the data in FIG. 3 and FIG. 4A-B.

### The Synthetic CAAP Oligopeptide Interacts with the Pre-Selected Target Protein Sequence

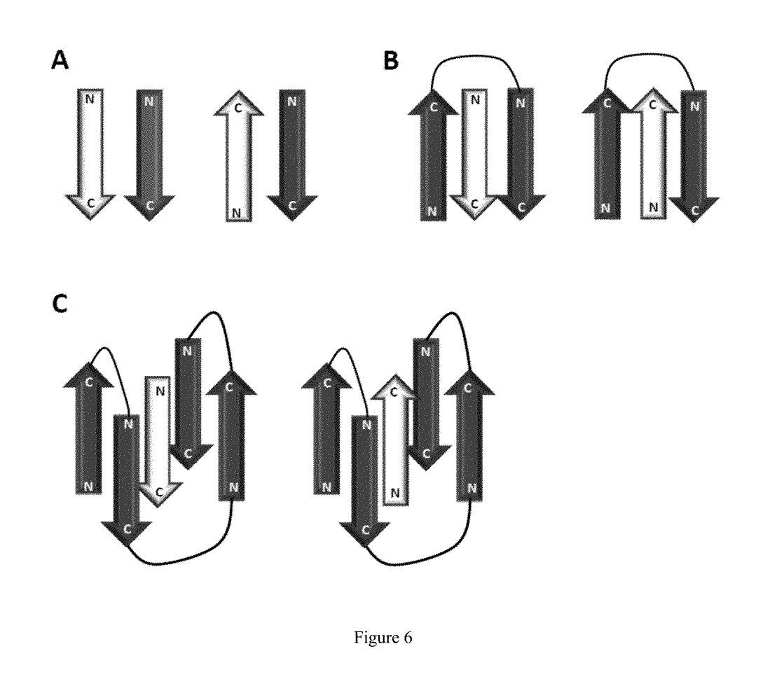

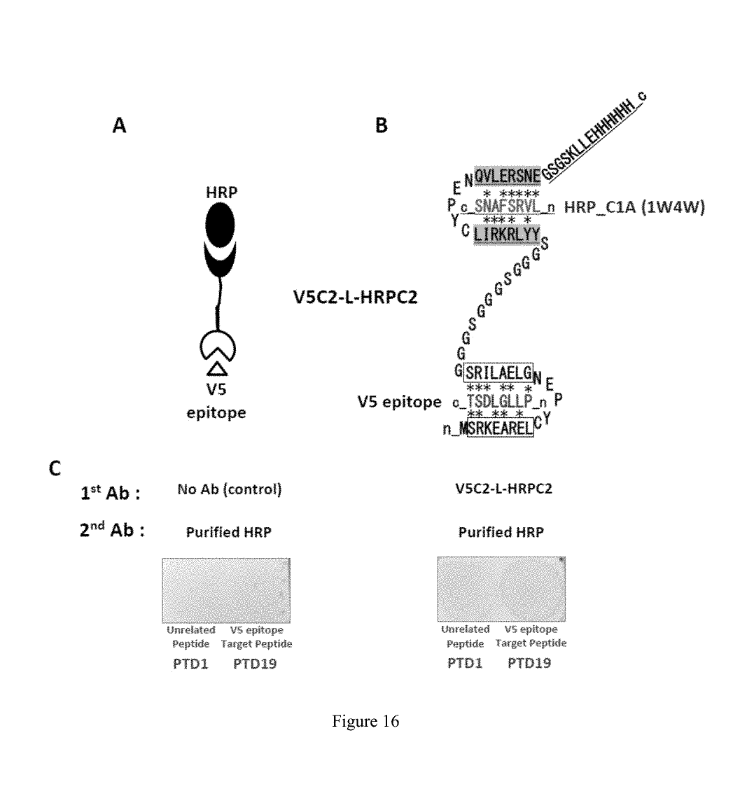

To test our CAAP design system, we selected target sequences in the three different proteins: Streptococcus pyogenes Cas9 [PDB_5B2R]; Escherichia coli alkaline phosphatase (AP) [PDB_3TG0]; Human Platelet-Derived Growth Factor (PDGF)/PDGF Receptor (PDGFR) complex [PDB_3MJG], and Horseradish Peroxidase plus V5 epitope (FIG. 16A-B). S. pyogenes CRISPR-Cas9 system has been broadly applied to edit the genome of bacterial and eukaryotic cells. PDGF/PDGFR is known as an important target for antitumor and antiangiogenic therapy. The target sequences for the Cas9, AP, and PDGF-B proteins are n_EKLYLYYLQ_c (SEQ ID NO: 26) (Helix: E813 to Q821), n_LVAHVTSRKC_c (SEQ ID NO: 21) (coil-beta sheet-coil: E159 to C168), and n_IEIVRKKPIF_c (SEQ ID NO: 23) (beta sheet: 1136 to F145), respectively. We designed four different types (monomer, dimer, and tetramer) of oligopeptides to detect the target protein sequences (FIG. 6A-C, FIG. 16A-B).

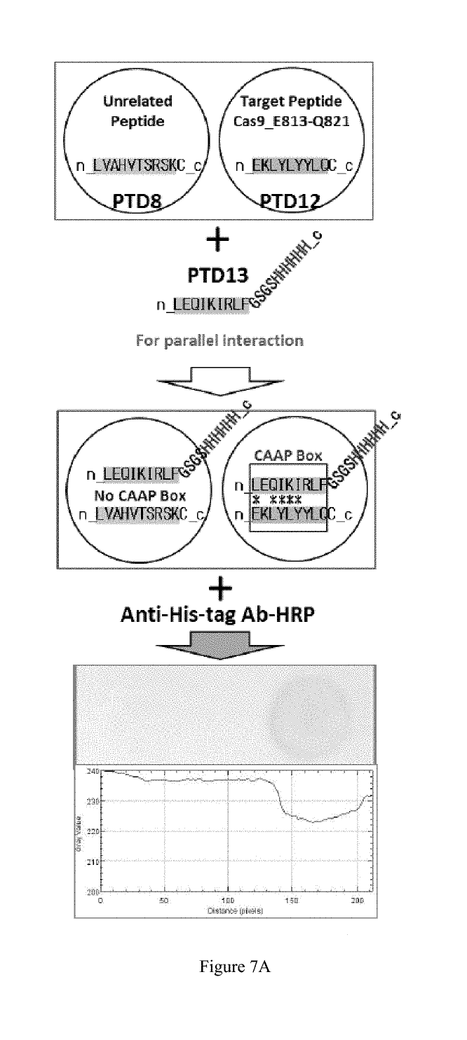

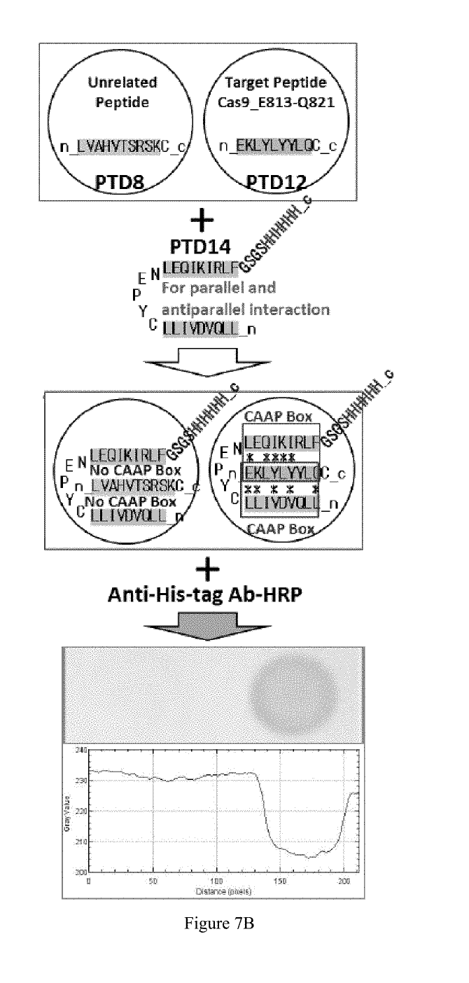

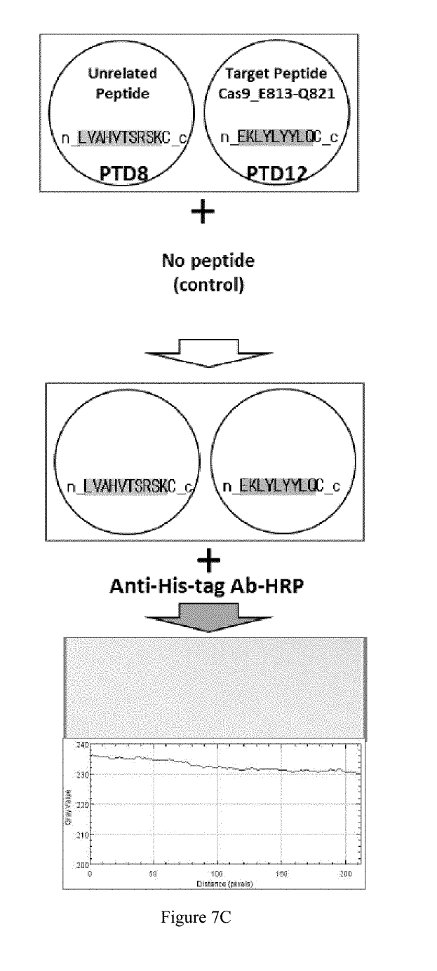

First, we performed a dot blot experiment to detect a Cas9 target sequence (PTD12 (SEQ ID NO: 27)) using the His-tagged CAAP oligopeptides, PTD13 (SEQ ID NO: 28) and PTD14 (SEQ ID NO: 11), (FIG. 7A-C). PTD8 (SEQ ID NO: 21) was used as an unrelated target (negative control). The synthetic CAAP oligopeptides, monomer (PTD13 (SEQ ID NO: 28)) and dimer (PTD14 (SEQ ID NO: 11)), could interact with the target peptide (PTD12 (SEQ IDNO: 27)), but no interaction with the control peptide (PTD8 (SEQ ID NO: 21)) was detected (FIG. 6A-6B). No signal was detected from the no peptide control (FIG. 7C). Remarkably, the CAAP oligopeptide dimer (PTD14 (SEQ ID NO: 11)) showed a stronger (two-fold) interaction than that of the monomer PTD13 (SEQ ID NO: 28).

Dual detection using a purified polypeptide V5C2-L-HRPC2 with two CAAP box dimer arms designed to interact with V5 epitope and HRP was also achieved. The V5C2-L-HRPC2 was designed with dual CAAP dimers to detect V5 epitope and HRP. Dot blot analysis using synthetic polypeptides, PTD1 (SEQ ID NO: 14) (unrelated, control) and immobilized PTD19 (SEQ ID NO: 32) (part of V5 epitope), as target molecules in presence or absence of V5C2-L-HRPC2 showed that the first interaction between immobilized V5 epitope and V5C2-L-HRPC2 was required for the second interaction between V5C2-L-HRPC2 and purified HRP protein. The interactions were visualized using a HRP chromogenic substrate (FIG. 16C).

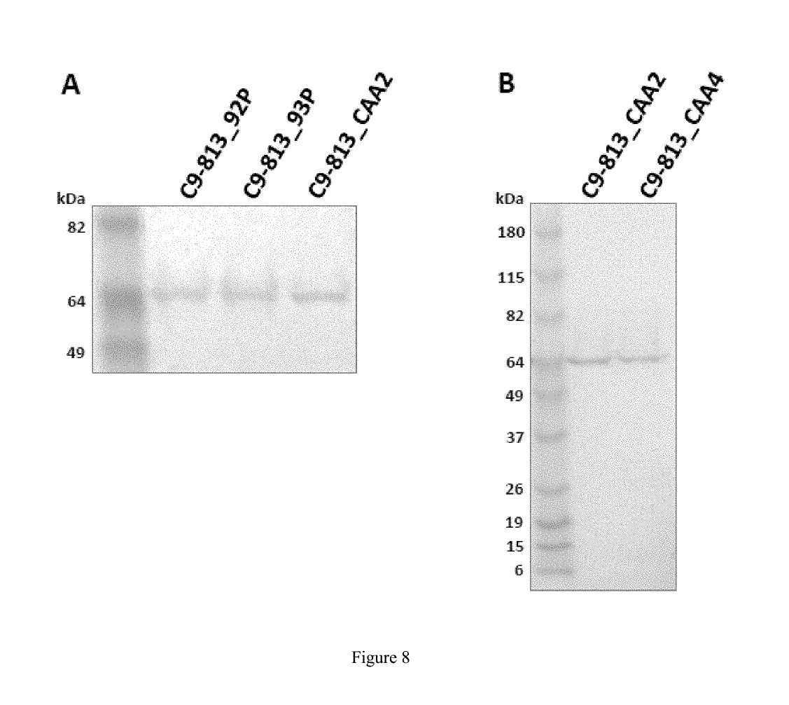

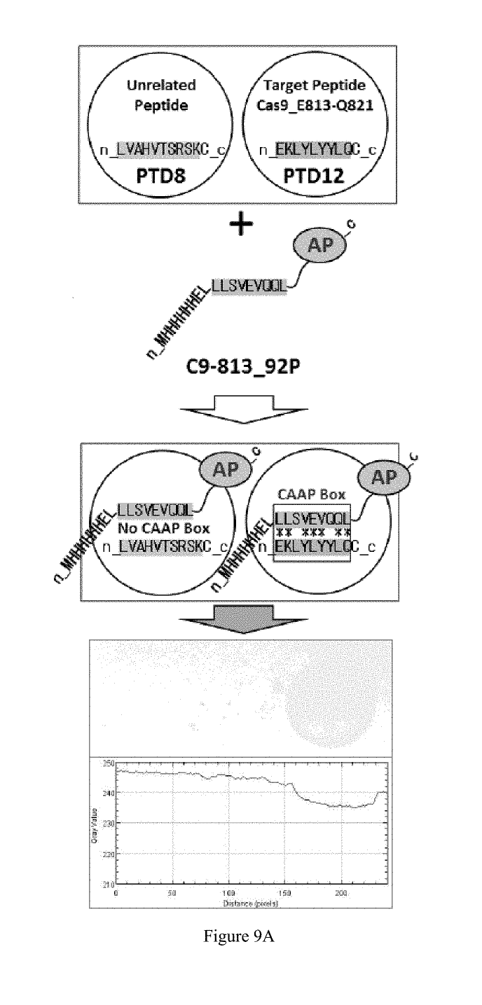

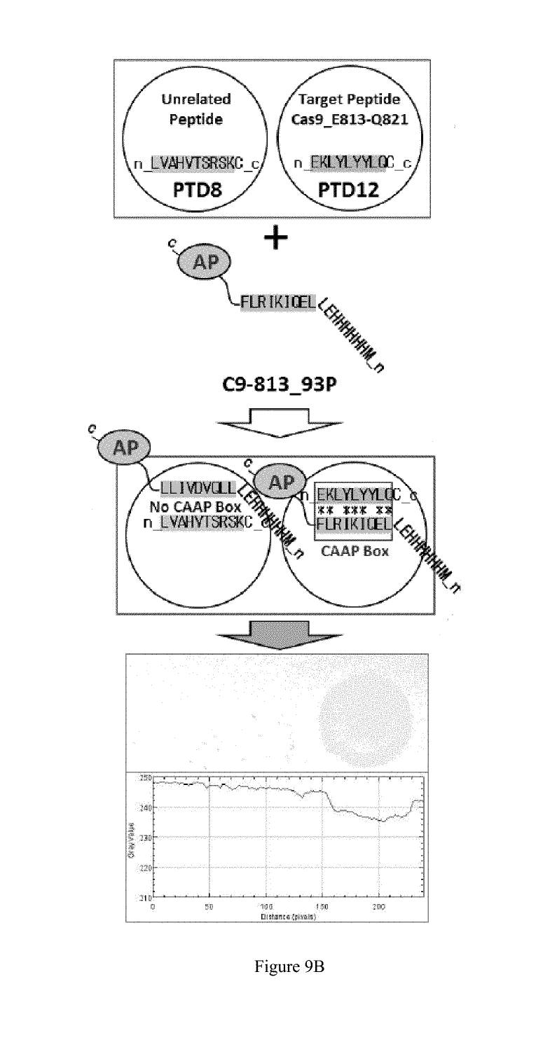

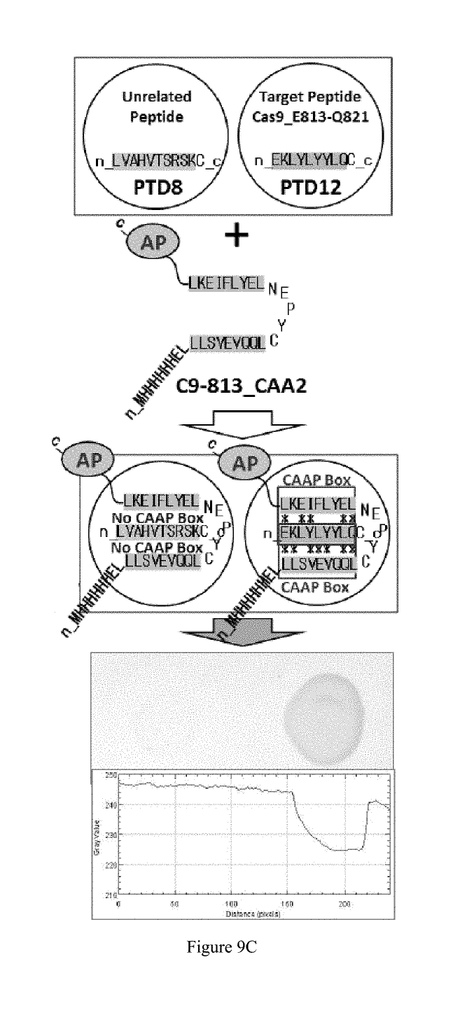

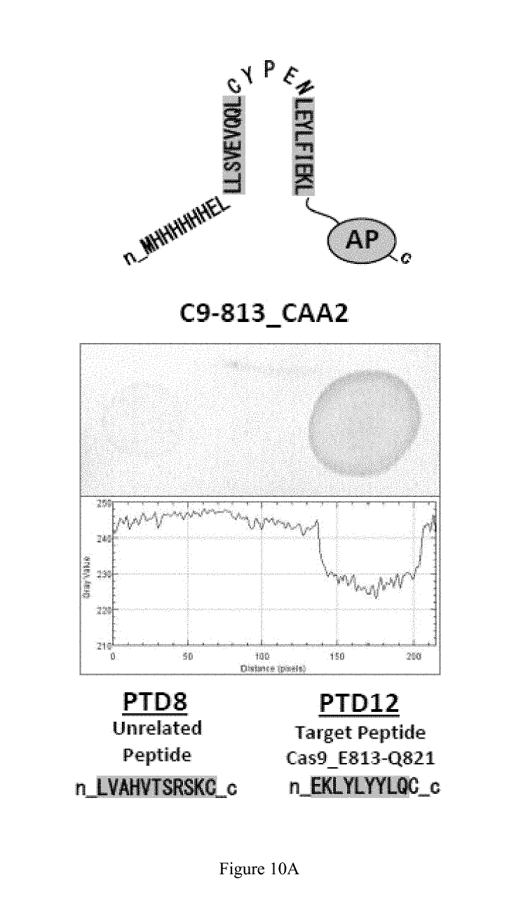

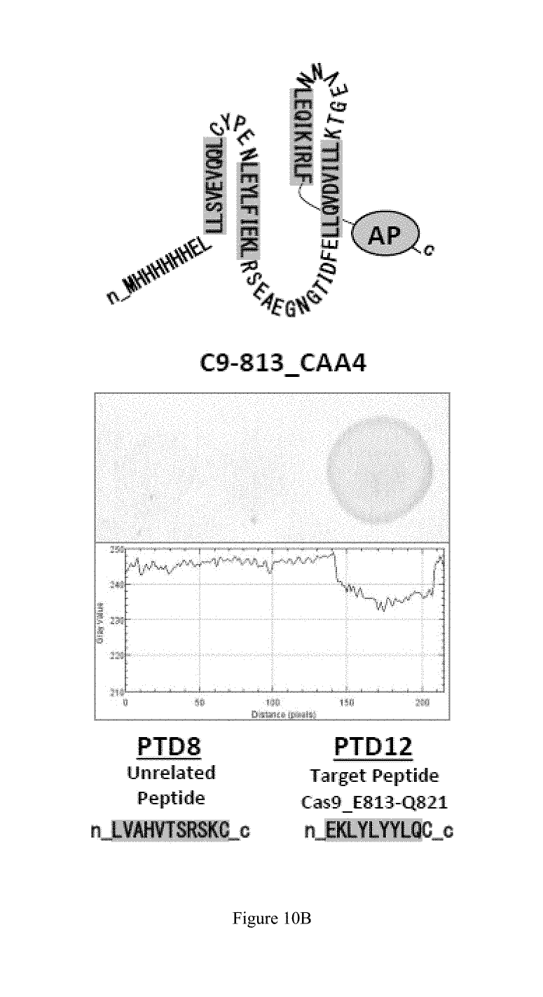

To verify these results, we produced three recombinant fusion proteins, C9-813-92P (monomer, parallel), C9-813-93P (monomer, antiparallel), and C9-813-CAA2 (dimer, antiparallel and parallel), that consist of the N-terminal His-tag (for purification), CAAP oligopeptide, and alkaline phosphatase (AP). Then the same amount of the purified proteins (FIG. 8A) was used for the dot blot experiments. All three CAAP oligopeptide-AP fusion proteins bound to the target peptide (PTD12 (SEQ ID NO: 27)), whereas none of them interacted with the unrelated control peptide (PTD8 (SEQ ID NO: 21)) (FIG. 9A-C). We confirmed that the dimer construct C9-813-CAA2 has stronger (2.5-fold) interaction with the Cas9 target sequence (PTD12 (SEQ ID NO: 27)) than the C9-813-92P (monomer, parallel) or C9-813-93P (monomer, antiparallel). We also compared the binding strength of the C9-813-CAA2 (dimer) and C9-813-CAA4 (tetramer) (FIG. 10A-B). Again, the same amount of the purified proteins (FIG. 8A-B) was used. Interestingly, the dimer interaction was 1.5-fold stronger than the tetramer interaction. Although the tetramer interaction was 1.5-fold weaker than the dimer interaction, it was still 1.5-fold stronger than the monomer interactions (FIG. 9A-B).

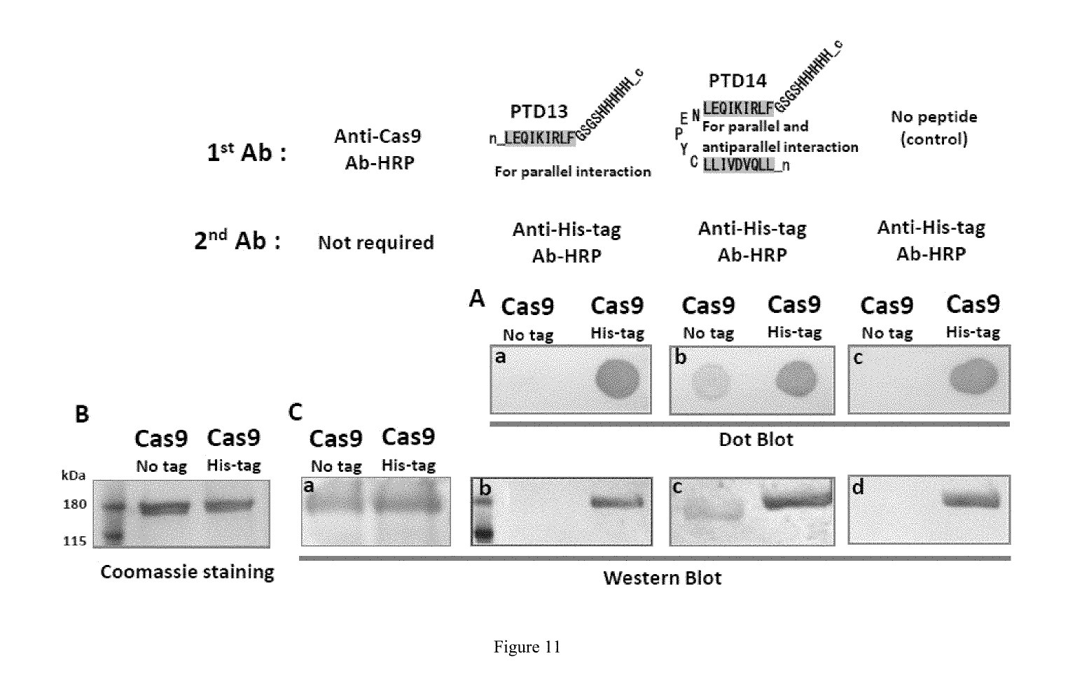

Finally, we further examined the performance of the CAAP oligopeptides to detect the whole Cas9 protein in both non-denatured (dot blot) and denatured (western blot) conditions. We used two different forms of the Cas9 protein: the Cas9 protein without any tag (no tag) as an actual target and the His-tagged Cas9 protein as a positive control. The purified Cas9 proteins are shown in FIG. 11B. We tested two synthetic His-tagged CAAP oligopeptides, monomer (PTD13 (SEQ ID NO: 28)) and dimer (PTD14 (SEQ ID NO: 11)), to detect Cas9 protein. No peptide (buffer) was used as negative control in both dot blot (FIG. 11Ac) and western blot experiments (FIG. 11Cd). The anti-Cas9 Ab-HRP conjugate was used as positive control in the western blot experiment (FIG. 11Ca). The synthetic His-tagged oligopeptide dimer (PTD14 (SEQ ID NO: 11)) was able to detect the Cas9 (no tag) protein in both the dot blot and western blot, while the monomer and the no peptide (negative control) were unable to detect the Cas9 (no tag) protein, suggesting that in at least some cases dimeric CAAP oligopeptides may be preferred.

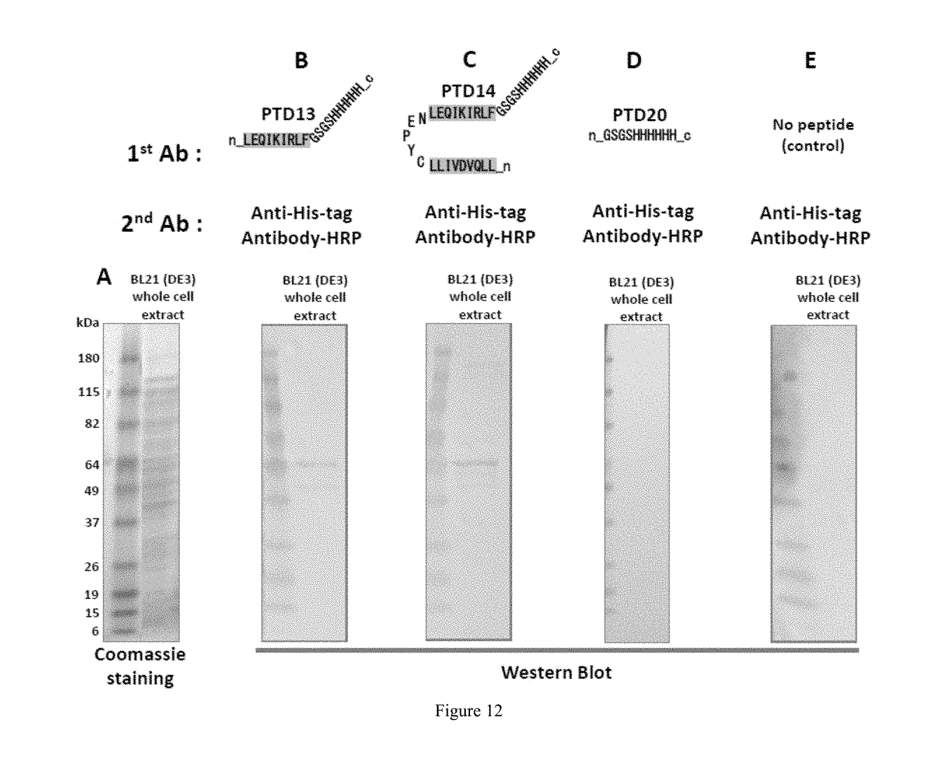

To evaluate the specificity of the synthetic CAAP oligopeptides, PTD13 (SEQ ID NO: 28) and PTD14 (SEQ ID NO: 11), we used them to detect any potential target in the whole proteome of E. coli BL21 Star DE3 (FIG. 12). The BL21 (DE3) strain has 4156 proteins (1,298,178 amino acids) according to UniProt [www.uniprot.org]. In our pilot search for CAAP boxes in BL21 proteins using a program developed in this study, we found multiple potential CAAP boxes. In the western blot experiment, however, both PTD13 (SEQ ID NO: 28) and PTD14 (SEQ ID NO: 11) detected only one major band and 6 minor bands (2 by PTD13 (SEQ ID NO: 28), 4 by PTD14 (SEQ ID NO: 11)) (FIG. 12). We believe that this is due to the large variation in the quality of the CAAP box, which we established to be having the most favorable CAAP and spacing according to our data (FIGS. 3 and 4A-B). In nature, thus, the probability of making a perfect CAAP box with 8 pairs of amino acids is very low. Therefore, a peptide having a CAAP box with 8 pairs of amino acids or more is unlikely to occur in nature.

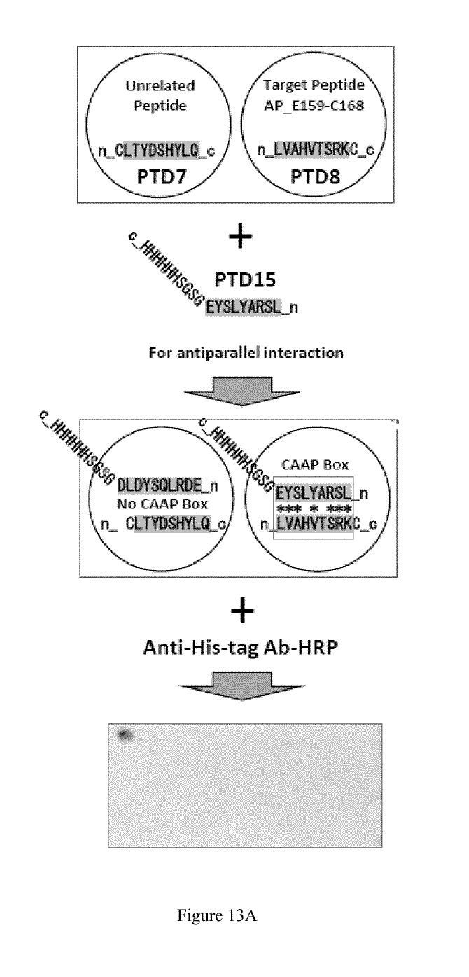

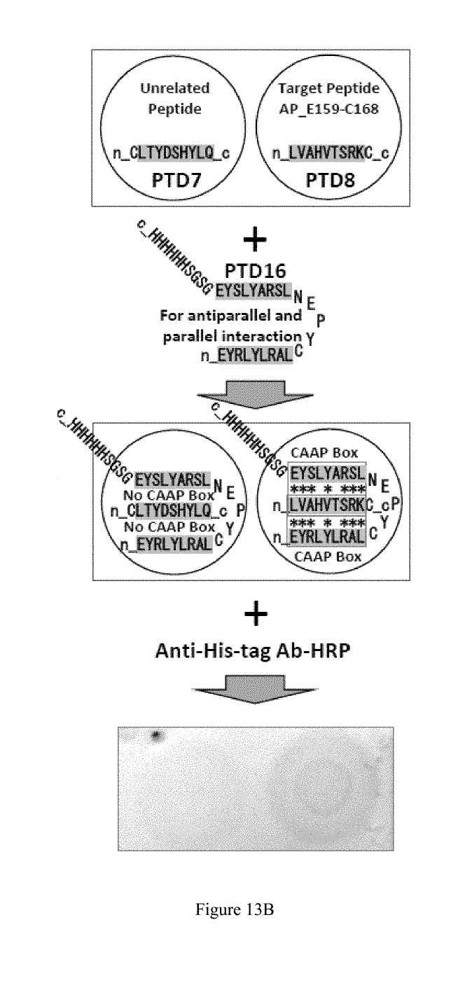

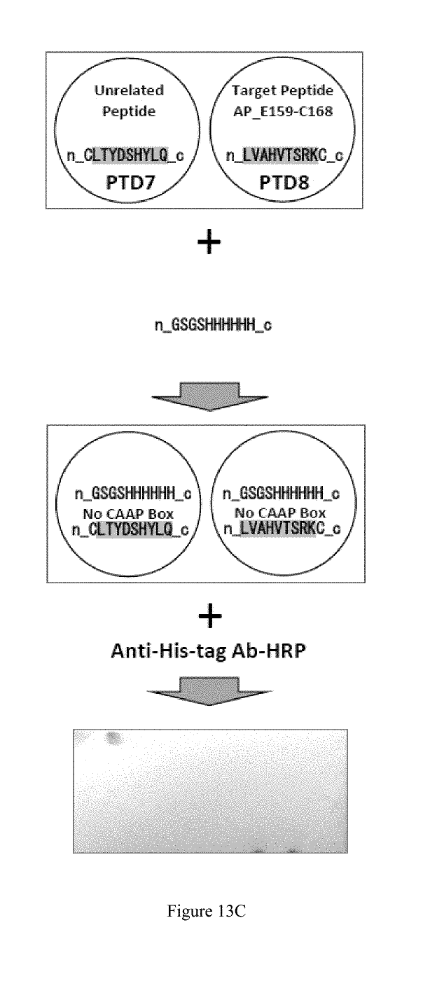

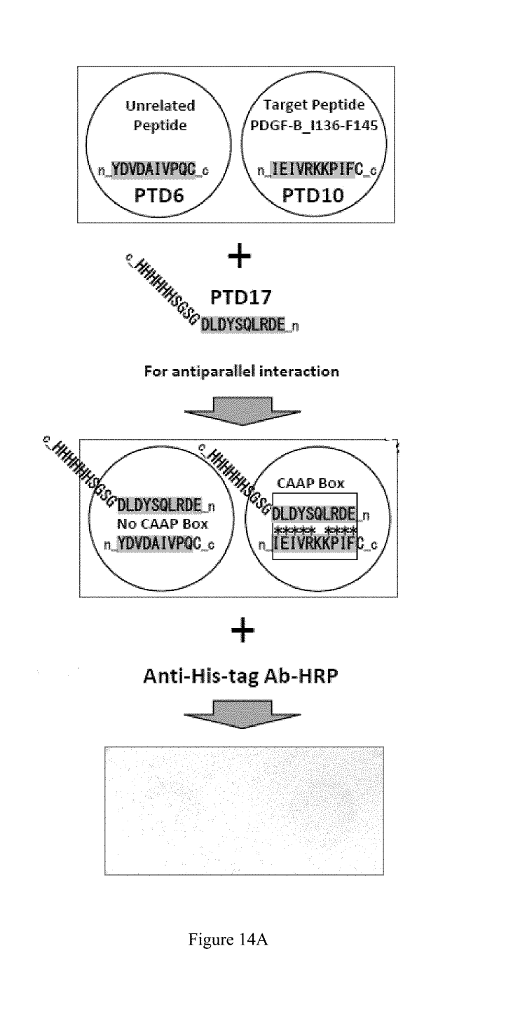

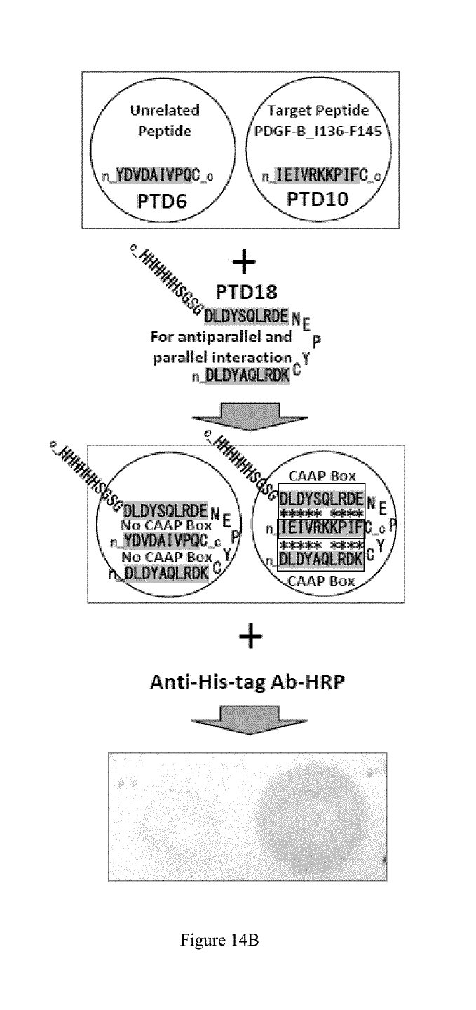

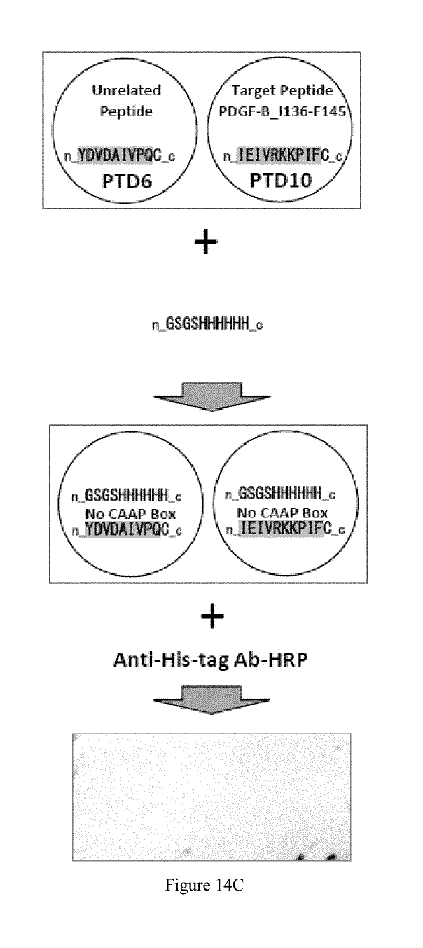

To investigate whether the CAAP-base protein interaction might be applicable for detecting the β-sheet structure, we designed CAAP oligopeptides to interact with two more target oligopeptide sequences: n_LVAHVTSRKC_c (SEQ ID NO: 21) (PTD8 (SEQ ID NO: 21), coil-beta sheet-coil) in the AP and n_IEIVRKKPIF_c (SEQ ID NO: 23) (PTD10 (SEQ ID NO: 24), beta sheet) in the PDGF-β. We first tested two synthetic His-tagged CAAP oligopeptides, PTD15 (SEQ ID NO: 29) (monomer, antiparallel) and PTD16 (SEQ ID NO: 30) (dimer, parallel and antiparallel), to detect the synthetic oligopeptide PTD8 (SEQ ID NO: 21) (FIG. 13A-C). The PTD7 (SEQ ID NO: 20) was used as an unrelated target peptide, which should not have a CAAP interaction with the PTD15 (SEQ ID NO: 29) or PTD16 (SEQ ID NO: 30). The PTD20 (SEQ ID NO: 289) (linker-His-tag only) was used as negative control. The PTD16 (SEQ ID NO: 30) (dimer) bound to the target (FIG. 13B), but the PTD15 (SEQ ID NO: 29) (monomer) and PTD20 (SEQ ID NO: 289) showed no detectable interaction with the target (FIG. 13A-C). Next we tested two synthetic His-tagged CAAP oligopeptides, PTD17 (SEQ ID NO: 13) (monomer, antiparallel) and PTD18 (SEQ ID NO: 31) (dimer, parallel and antiparallel), to detect the synthetic oligopeptide PTD10 (SEQ ID NO: 24) (FIG. 14A-C). The PTD6 (SEQ ID NO: 19) was used as unrelated target peptide, which cannot have CAAP interaction with the PTD17 (SEQ ID NO: 13) or PTD18 (SEQ ID NO: 31). The PTD18 (SEQ ID NO: 31) (dimer) bound to the target (FIG. 14B), but the PTD17 (SEQ ID NO: 13) (monomer) and PTD20 (SEQ ID NO: 289) (negative control) showed no detectable interaction with the target (FIG. 14A-C).

The CAAP oligopeptide PTD14 induces non-specific DNA binding activity of the Cas9 nuclease

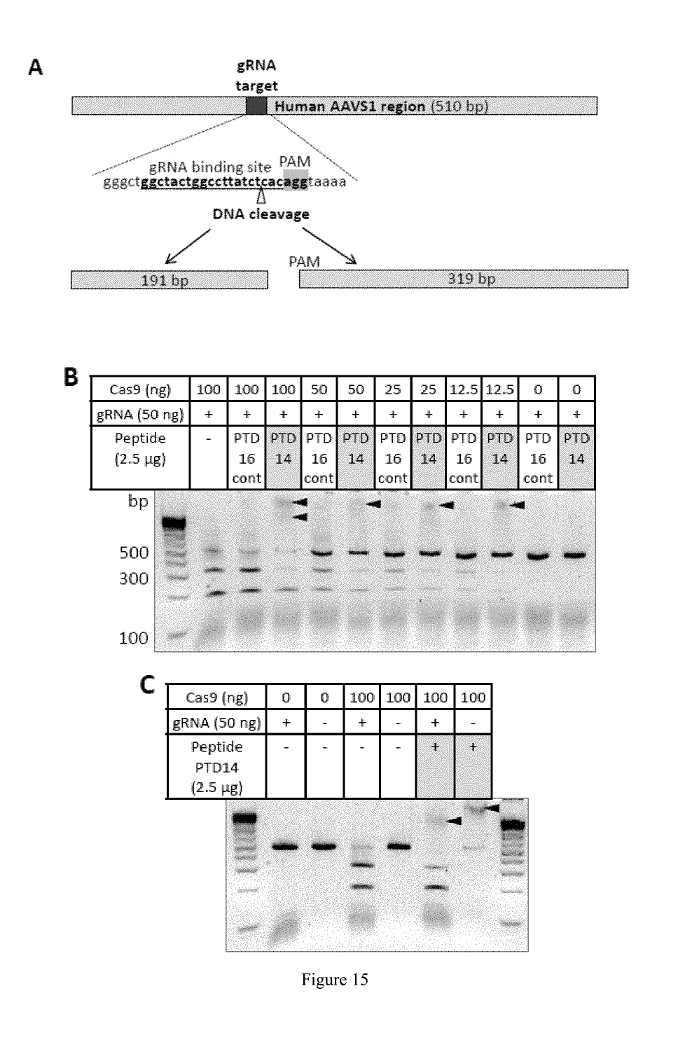

The PTD14 (SEQ ID NO: 11) target site [E813 to Q821] in the Cas9 protein is located in the HNH domain, which is important for DNA binding and DNA cleavage by conformational change. Thus we first tested the effect of the PTD14-Cas9 (SEQ ID NO: 11) interaction on the RNA-guided DNA cleavage by Cas9 nuclease. The PTD16 (SEQ ID NO: 30) was used as negative control. We used a 510 bp human AAV1 region as a target DNA and in vitro transcribed gRNA. We designed a gRNA specific for the AAVS1 to produce 191bp and 319 bp DNA cleavage products (FIG. 15A). Interestingly, although PTD14 (SEQ ID NO: 11) showed no significant effect on DNA cleavage, it directed very strong non-specific DNA binding activity of the Cas9 protein (FIG. 15B-C).

### Materials and Methods

### Oligonucleotides, Synthetic DNA, Synthetic Peptides, and Enzymes

Oligonucleotides were obtained from Integrated DNA Technologies (IDT) and Thermo Fisher Scientific, and listed in Table 1. Synthetic DNA fragments were obtained from IDT DNA, and listed in Table 1. Synthetic peptides were purchased from Peptide 2.0 and listed in Table 1. Restriction enzymes and DNA modifying enzymes were purchased from New England Biolabs (NEB) and Thermo Fisher Scientific. The purified horseradish peroxidase (HRP) was obtained from PROSPEC.

### Generation of Expression Vectors for the Recombinant Proteins

The bacterial expression vector, pET-21b, was obtained from EMD Millipore (catalog # 69741-3). All plasmids were constructed by assembling two linear DNA fragments, vector and insert, with overlapping ends using a seamless DNA assembly method following the manufacturer's protocol [Thermo Fisher Scientific, GeneArt™ Seamless Cloning and Assembly Enzyme Mix, catalog # A14606]. Briefly, the pET-21b vector was digested with SwaI/XhoI, and assembled with a 143 bp DNA fragment, 92_6HNLS to produce vector pC9-813-92 or 93_6HNLS to produce vector pC9-813-93. The DNA fragments correspond to the parallel CAAP box and antiparallel CAAP box used to detect the Cas9 protein, respectively. The pC9-813-92 and pC9-813-93 vectors were digested with BamHI, and assembled with a 1501 bp DNA fragment 92P or 93P, corresponding to the E. coli alkaline phosphatase (AP) fusion, to generate pC9-813-92P and pC9-813-93P, respectively. The pC9-813-92P vector was digested with BgIII, assembled with a 204 bp synthetic DNA fragment Sp-C9_813-821_CAA, corresponding to the CAAP box tetramer used to detect Cas9, to generate pC9-813-CAA4. The pC9-813-CAA4 vector was digested with BgIII, and self-ligated (to remove 117 bp DNA fragment encoding two CAAP boxes), producing pC9-813-CAA2 which corresponds to the CAAP box dimer to used detect Cas9. A 258 bp synthetic DNA fragment V5C2-L-HRPC2, corresponding to the dual CAAP box dimer arms used to detect both V5 epitope and HRP, was assembled with the SwaI/XhoI-digested pET-21b to generate pV5C2-L-HRPC2.

For production of the recombinant Cas9 proteins, the pET-Spy-Cas9_6His and pET-Spy-Cas9_d6H vectors were constructed by assembling five parts with overlapping DNA ends using the seamless DNA assembly kit. Briefly, four insert parts [a 1000 bp Spy-Cas9_1, a 1030 bp Spy-Cas9_2, a 1030 bp Spy-Cas9_3, and a 1300 bp Spy-Cas9_4, corresponding to the His-tagged Cas9] and the SwaI/XhoI-digested pET-21b were assembled, to create pET-Spy-Cas9_6His. Similarly, four insert parts [a 1000 bp Spy-Cas9_1, a 1030 bp Spy-Cas9_2, a 1030 bp Spy-Cas9_3, and a 1303 bp Spy-Cas9_5, corresponding to the tagless Cas9] and the SwaI/XhoI-digested pET-21b were assembled, to create pET-Spy-Cas9_d6H.

### Bacterial strains

The E. coli strain DH10B T1 [Thermo Fisher Scientific, catalog # 12331013] was used as a cloning host. The E. coli strain BL21 Star (DE3) [Thermo Fisher Scientific, catalog # C601003] was used for production of the recombinant proteins.

### Protein Purification

For the recombinant protein production, the BL21 Star (DE3) cells harboring an expression vector were grown to mid-log phase (optical density at 600 nm [0D600] of 0.6) in LB medium [ampicillin (Amp), 100 μg/ml] at 28° C. and induced with 1 mM IPTG (isopropyl-β-D-thiogalactopyranoside) for 5 h. Cells were harvested by centrifugation at 3000 rpm for 10 min. The harvested cells were disrupted by using a chemical lysis method following the manufacturer's protocol [Thermo Fisher Scientific, B-PER™ Complete Bacterial Protein Extraction Reagent, catalog # 89821]. Cell debris and insoluble proteins in the lysate were separated by centrifugation at 16,000×g for 5 minutes. The His-tagged recombinant proteins were purified by a metal-affinity chromatography using the Dynabeads™ His-Tag Isolation and Pulldown beads following the manufacturer's protocol [Thermo Fisher Scientific, catalog # 10103D].

The recombinant Cas9 proteins were purified using the HiTrap heparin HP column [GE Healthcare, catalog # 17-0406-01] as previously described (Karvelis et al., 2015).

### CRISPR-Cas9 Single Guide RNA (sgRNA) Synthesis

The sgRNA targeting human AAVS1 region (target sequence GGCTACTGGCCTTATCTCACAGG (SEQ ID NO: 36), PAM sequence underlined) was synthesized by in vitro transcription using a 118 bp PCR-assembled DNA fragment AAVS1_T23826 as template, following the manufacturer's protocol [Thermo Fisher Scientific, TranscriptAid T7 High Yield Transcription Kit, catalog # K0441]. The sgRNA product was purified using the GeneJET RNA Purification Micro Column [Thermo Fisher Scientific, catalog # K0841].

### Dot Blot and Western Blot Analysis

For dot blot analysis, 1 μl (2.5 μg) or 2 μl (5 μg) of samples were spotted onto the nitrocellulose (NC) membrane and dried completely. Then, non-specific sites were blocked by soaking the membrane in the blocking solution made for NC membranes [Thermo Fisher Scientific, WesternBreeze™ Blocker/Diluent (Part A and B), catalog # WB7050]. The membrane was washed twice with water (1 ml per cm² membrane), and incubated with the 1^{st} antibody (Ab) in a binding/wash (BW) buffer [50 mM sodiumphosphate, pH 8.0, 300 mM NaCl, and 0.01% Tween 20] for 1 h. The membrane was washed 4 times (for 2 minutes per wash) with the wash buffer [Thermo Fisher Scientific, WesternBreeze™ Wash Solution, catalog # WB7003]. If the 1^{st} oligopeptide was Anti-Cas9 Ab-HRP conjugate [Thermo Fisher Scientific, catalog # MAC133P] or the peptide-AP fusions, the membrane was washed twice with water, and incubated with the chromogenic substrates, Chromogenic Substrate (TMB) [Thermo Fisher Scientific, catalog # WP20004] for HRP and NBT/BCIP substrate solution for AP [Thermo Fisher Scientific, catalog # 34042]. Otherwise, the membrane was incubated with in the blocking solution for 1 h. To detect His-tagged peptide and proteins, the Anti-6His Ab-HRP conjugate [Thermo Fisher Scientific, catalog 46-0707] was used. Then the membrane was washed four times with the wash buffer and two times with water. Finally, the blot was incubated with the chromogenic substrates.

For the western blot analysis, the protein samples were resolved in 4-20% gradient SDS-PAGE gel, transferred to NC membrane, and subjected to the western blot analysis using the same method for the dot blot analysis.

### Cas9 Activity Assay In Vitro

A 510 bp human AAVS1 region was amplified from HEK293 genomic DNA by PCR using a primer set (CH1161 and CH1162) and used as a target DNA for the in vitro CRISPR/Cas9 assay. Performance of the Cas9 protein was assessed in various concentrations of Cas9 [100, 50, 25, 12.5, and 0 ng] in presence or absence of sgRNA and peptides (PTD14 (SEQ ID NO: 11) and PTD16 (SEQ ID NO: 30)) in the 1×buffer K [20 mM Tris-HCl, pH 8.5, 10 mM MgCl2, 1 mM Dithiothreitol (DTT), and 100 mM KCl]. The PTD16 (SEQ ID NO: 30) was used as an unrelated peptide control. The reaction mixture was incubated at 37° C. for 15 minutes. The reaction was stopped by adding a stop buffer [1 mM Tris-HCl (pH 7.5), 10 mM EDTA, 6.5% (w/v) Sucrose, 0.03% (w/v) Bromophenol Blue] and heat inactivated at 75° C. for 5 minutes. The reaction samples were resolved in 4% agarose gel.

TABLE 1

| Target Amino Acid | Corresponding Amino Acid for Binding Peptide |
|---|---|
| N | I, V |
| Y | I, V |
| C | T, A |
| S | R, G, T, A |
| T | S, G, C, R |
| Q | L |
| W | P |
| I | N, D, Y |
| M | H |
| P | R, G, W |
| F | K, E |
| G | T, A, S, P |
| A | S, G, C, R |
| V | N, D, Y, H |
| L | Q, K, E |
| H | M, V |
| E | F, L |
| R | T, A, S, P |
| K | F, L |
| D | I, V |

TABLE 2
Primers used in this study

| | | Related DNA |
|---|---|---|
| Name | Sequence (5′ to 3′) | fragment(s) |
| CH1149 | taatacgactcactatagggctactggccttat (SEQ ID NO: 37) | AAVS1_T23826 |
| CH1150 | TTCTAGCTCTAAAACgtgagataaggccagtagcc (SEQ ID NO: 38) | AAVS1_T23826 |
| CH1161 | ggaggaatatgtcccagatag (SEQ ID NO: 39) | AAVS1 |
| CH1162 | AAGGTTTGCTTACGATGGAG (SEQ ID NO: 40) | AAVS1 |
| CH1389 | ccctctagaatagaaggagatttaaatgcaccatcaccaccatcacGAGCTC (SEQ ID | 92_6HNLS and |
| | NO: 41) | 93_6HNLS |
| CH1392 | TCAGGATCCTTACAGCTGCTGAACTTCAACGCTCAGCAGGAGC | 92_6HNLS |
| | TCGTGATGGTGGTGATG (SEQ ID NO: 42) | |
| CH1393 | TCAGGATCCTTAAAACAGACGGATTTTAATCTGCTCTAAGAGC | 93_6HNLS |
| | TCGTGATGGTGGTGATG (SEQ ID NO: 43) | |
| CH1405 | GGACTTTGCGTTTCTTTTTCGGATC (SEQ ID NO: 44) | 92P and 93P |
| CH1424 | agcgttgaagttcagcagctgagatctgtgaaacaaagcactattg (SEQ ID NO: 45) | 92P |
| CH1425 | cagattaaaatccgtctgtttagatctgtgaaacaaagcactattg (SEQ ID NO: 46) | 93P |
| CH1496 | agccggatctcagtggtggtggtggtggtgctcgaggactttgcgtttctttttcggatcctta (SEQ ID | 92_6HNLS and |
| | NO: 47) | 93_6HNLS |
| CH1497 | AAAAGCACCGACTCGGTG (SEQ ID NO: 48) | AAVS1_T23826 |

TABLE 3
DNA fragments used in this study

| Name | Sequence (5′ to 3′) | Production |
|---|---|---|
| 92_6HNLS | ccctctagaatagaaggagatttaaatgcacCATCACCACCATCACGAGCTCCTGCT | PCR |
| | GAGCGTTGAAGTTCAGCAGCTGTAAGGATCCgaaaaagaaacgcaaagtcctc | |
| | gagcaccaccaccaccaccactgagatccggct (SEQ ID NO: 49) | |
| 93_6HNLS | ccctctagaatagaaggagatttaaatgcacCATCACCACCATCACGAGCTCTTAGA | PCR |
| | GCAGATTAAAATCCGTCTGTTTTAAGGATCCgaaaaagaaacgcaaagtcctc | |
| | gagcaccaccaccaccaccactgagatccggct (SEQ ID NO: 50) | |
| Sp-C9_813- | AGCGTTGAAGTTCAGCAGCTGTGCTATCCGGAAAACCTCGAATAC | Synthetic |
| 821_CAA | CTGTTTATTGAAAAATTAAGATCTGAAGCCGAAGGCAACGGCACT | |
| | ATAGACTTCGAGCTCCTGTTACAGGTGGATGTGATTCTGCTCAAA | |
| | ACCGGTGAAGTCAACAACTTAGAGCAGATTAAAATCCGTCTGTTT | |
| | AGATCTGTGAAACAAAGCACTATT (SEQ ID NO: 51) | |
| 92P | agcgttgaagttcagcagctgagatctgtgaaacaaagcactattgcactggcactcttaccgttactgtttacc | PCR |
| | cctgtgacaaaagcccggacaccagaaatgcctgttctggaaaaccgggctgctcagggcgatattactgca | |
| | cccggcggtgctcgccgtttaacgggtgatcagactgccgctctgcgtgattctcttagcgataaacctgcaaa | |
| | aaatattattttgctgattggcgatgggatgggggactcggaaattactgccgcacgtaattatgccgaaggtgc | |
| | gggcggcttttttaaaggtatagatgcctcaccgcttaccgggcaatacactcactatgcgctgaataaaaaaa | |
| | ccggcaaaccggactacgtcaccgactcggctgcatcagcaaccgcctggtcaaccggtgtcaaaacctat | |
| | aacggcgcgctgggcgtcgatattcacgaaaaagatcacccaacgattctggaaatggcaaaagccgcagg | |
| | tctggcgaccggtaacgtttctaccgcagagttgcaggatgccacgcccgctgcgctggtggcacatgtgac | |
| | ctcgcgcaaatgctacggtccgagcgcgaccagtgaaaaatgtccgggtaacgctctggaaaaaggcgga | |
| | aaaggatcgattaccgaacagctgcttaacgctcgtgccgacgttacgcttggcggcggcgcaaaaacctttg | |
| | ctgaaacggcaaccgctggtgaatggcagggaaaaacgctgcgtgaacaggcacaggcgcgtggttatca | |
| | gttggtgagcgatgctgcctcactgaattcggtgacggaagcgaatcagcaaaaacccctgcttggcctgttt | |
| | gctgacggcaatatgccagtgcgctggctaggaccgaaagcaacgtaccatggcaatatcgataagcccgc | |
| | agtcacctgtacgccaaatccgcaacgtaatgacagtgtaccaaccctggcgcagatgaccgacaaagccat | |
| | tgaattgttgagtaaaaatgagaaaggctttttcctgcaagttgaaggtgcgtcaatcgataaacaggatcatgc | |
| | tgcgaatccttgtgggcaaattggcgagacggtcgatctcgatgaagccgtacaacgggcgctggaattcgct | |
| | aaaaaggagggtaacacgctggtcatagtcaccgctgatcacgcccacgccagccagattgttgcgccgga | |
| | taccaaagctccgggcctcacccaggcgctaaataccaaagatggcgcagtgatggtgatgagttacggga | |
| | actccgaagaggattcacaagaacataccggcagtcagttgcgtattgcggcgtatggcccgcatgccgcca | |
| | atgttgttggactgaccgaccagaccgatctcttctacaccatgaaagccgctctggggctgaaagcttccgg | |
| | ctctagccatcaccatcaccatcacggttcatctgcggatccgaaaaagaaacgcaaagtcctcgagcacca | |
| | ccaccaccaccactga (SEQ ID NO: 52) | |
| 93P | cagattaaaatccgtctgtttagatctgtgaaacaaagcactattgcactggcactcttaccgttactgtttacccc | PCR |
| | tgtgacaaaagcccggacaccagaaatgcctgttctggaaaaccgggctgctcagggcgatattactgcacc | |
| | cggcggtgctcgccgtttaacgggtgatcagactgccgctctgcgtgattctcttagcgataaacctgcaaaaa | |
| | atattattttgctgattggcgatgggatgggggactcggaaattactgccgcacgtaattatgccgaaggtgcg | |
| | ggcggcttttttaaaggtatagatgcctcaccgcttaccgggcaatacactcactatgcgctgaataaaaaaac | |
| | cggcaaaccggactacgtcaccgactcggctgcatcagcaaccgcctggtcaaccggtgtcaaaacctata | |
| | acggcgcgctgggcgtcgatattcacgaaaaagatcacccaacgattctggaaatggcaaaagccgcaggt | |
| | ctggcgaccggtaacgtttctaccgcagagttgcaggatgccacgcccgctgcgctggtggcacatgtgacc | |
| | tcgcgcaaatgctacggtccgagcgcgaccagtgaaaaatgtccgggtaacgctctggaaaaaggcggaa | |
| | aaggatcgattaccgaacagctgcttaacgctcgtgccgacgttacgcttggcggcggcgcaaaaacctttgc | |
| | tgaaacggcaaccgctggtgaatggcagggaaaaacgctgcgtgaacaggcacaggcgcgtggttatcagt | |
| | tggtgagcgatgctgcctcactgaattcggtgacggaagcgaatcagcaaaaacccctgcttggcctgtttgct | |
| | gacggcaatatgccagtgcgctggctaggaccgaaagcaacgtaccatggcaatatcgataagcccgcagt | |
| | cacctgtacgccaaatccgcaacgtaatgacagtgtaccaaccctggcgcagatgaccgacaaagccattga | |
| | attgttgagtaaaaatgagaaaggctttttcctgcaagttgaaggtgcgtcaatcgataaacaggatcatgctgc | |
| | gaatccttgtgggcaaattggcgagacggtcgatctcgatgaagccgtacaacgggcgctggaattcgctaa | |
| | aaaggagggtaacacgctggtcatagtcaccgctgatcacgcccacgccagccagattgttgcgccggata | |
| | ccaaagctccgggcctcacccaggcgctaaataccaaagatggcgcagtgatggtgatgagttacgggaac | |
| | tccgaagaggattcacaagaacataccggcagtcagttgcgtattgcggcgtatggcccgcatgccgccaat | |
| | gttgttggactgaccgaccagaccgatctcttctacaccatgaaagccgctctggggctgaaagcttccggct | |
| | ctagccatcaccatcaccatcacggttcatctgcggatccgaaaaagaaacgcaaagtcctcgagcaccacc | |
| | accaccaccactga (SEQ ID NO: 53) | |
| Spy-Cas9_1 | ccctctagaatagaaggagatttaaatggataagaaatacagcattggtttggacattggtacgaatagcgttg | Synthetic |
| | gttgggcagtcattaccgacgagtacaaggtgccgagcaagaagtttaaagtattgggtaacacggaccgtc | |
| | acagcattaagaaaaacctgattggtgcactgctgtttgacagcggtgaaactgcagaggcgactcgcctgaa | |
| | gcgtaccgcgcgtcgccgctatactcgtcgtaaaaaccgtatctgctatctgcaggagatctttagcaacgaga | |
| | tggcgaaggttgatgacagcttctttcaccgtctggaagaaagcttcctggtcgaagaggacaaaaagcacg | |
| | agcgccatccgatcttcggcaacattgtggacgaagtggcttatcatgaaaagtatccgaccatttatcatctgc | |
| | gtaagaagctggttgatagcaccgataaagcggatctgcgtctgatttacctggcactggcccacatgatcaa | |
| | gtttcgcggccactttctgatcgagggtgatctgaatccggacaatagcgacgttgacaagctgttcatccaact | |
| | ggtccaaacgtacaaccagctgttcgaagaaaacccgatcaacgcgagcggtgtggatgcaaaagctattct | |
| | gagcgcgcgtctgagcaagagccgtcgtttggagaatctgatcgcgcaattgccgggtgagaagaaaaatg | |
| | gcctgttcggtaatctgattgcactgtccctgggcctgacgccgaacttcaaaagcaattttgatctggcagaag | |
| | atgcgaagctgcaactgagcaaagatacttatgatgacgacctggacaatctgttggcacaaatcggtgacca | |
| | gtatgcagatctgtttctggcggcaaagaacctgtccgatgcgatcctgctgagcgacattctgcgcgtgaaca | |
| | cggaaattaccaaggctccgctgagcgcgagcatgattaagcgttac (SEQ ID NO: 54) | |
| Spy-Cas9_2 | ccgctgagcgcgagcatgattaagcgttacgatgagcaccaccaggatctgaccctgctgaaggcgctggtc | Synthetic |
| | cgtcagcaactgccggaaaagtacaaagagattttctttgaccagagcaagaatggctacgcgggctatatcg | |
| | atggtggcgctagccaagaagagttctacaagtttatcaagccgattttggagaaaatggatggtaccgaaga | |
| | gttgctggttaaactgaatcgtgaagatctgctgcgtaagcaacgcacctttgataatggcagcattccgcatca | |
| | aattcacctgggtgagttgcatgctatcctgcgccgtcaagaggatttctacccgtttctgaaagacaaccgtga | |
| | gaagatcgagaaaattctgactttccgcatcccgtattacgtcggtccgctggcgcgtggtaacagccgtttcg | |
| | catggatgacccgtaagagcgaagaaaccatcaccccatggaacttcgaagaggttgtggataagggtgcat | |
| | ccgcgcaaagcttcatcgagcgtatgacgaattttgacaagaatctgccgaatgaaaaagtgctgccgaagc | |
| | acagcctgctgtacgaatactttaccgtctataacgagctgaccaaagtcaaatacgtcaccgagggtatgcgt | |
| | aaaccggcgttcctgagcggcgagcagaagaaggcgattgtcgatctgctgttcaaaacgaatcgtaaagtt | |
| | acggttaagcaactgaaagaggactacttcaagaaaattgaatgtttcgactctgtcgagattagcggtgttgaa | |
| | gatcgcttcaatgcgagcttgggtacctatcatgatctgctgaagatcatcaaagacaaagatttcctggataat | |
| | gaagagaacgaggacattctggaagatatcgttttgacgctgaccttgttcgaagatcgtgagatgatcgaag | |
| | aacgcctgaaaacgtatgcgcacctgtttgatgataaagtgatgaaacaactgaagcgtcgccgttataccggt | |
| | t (SEQ ID NO: 55) | |
| Spy-Cas9_3 | aacaactgaagcgtcgccgttataccggttggggtcgtctgagccgtaagctgatcaacggcattcgtgataa | Synthetic |
| | acagtccggtaagacgatcctggattttctgaaaagcgacggcttcgcaaaccgtaatttcatgcagctgattc | |
| | acgacgacagcttgaccttcaaagaggacatccagaaagcacaagttagcggtcaaggcgatagcctgcat | |
| | gagcacattgcaaatttggcgggtagcccagcgatcaagaagggtattctgcagaccgttaaagtggttgatg | |
| | aactggtgaaagttatgggccgtcacaagcctgaaaacatcgtcattgagatggcgcgtgaaaatcagacca | |
| | cgcaaaagggccagaagaatagccgtgaacgcatgaaacgtatcgaagagggcattaaagaactgggctc | |
| | ccaaatcctgaaagagcatccggtggagaatactcaactgcagaatgaaaagctgtacctgtactatctgcaa | |
| | aacggtcgcgatatgtacgtcgaccaggagctggacatcaaccgcctgtccgactatgacgttgatcacattg | |
| | tcccgcagagcttcctgaaagatgacagcatcgacaacaaggtcctgacccgtagcgataagaatcgcggta | |
| | aaagcgataacgtgccaagcgaagaagtggtgaagaagatgaaaaactattggcgtcaactgttgaacgcta | |
| | aattgattacgcaacgtaagttcgacaacctgaccaaggcggaacgtggtggcctgagcgaactggacaaa | |
| | gcgggtttcatcaagcgccaactggtggaaacccgtcagattacgaaacatgtcgcccaaattctggacagc | |
| | cgtatgaacacgaagtacgatgaaaacgataaactgattcgtgaagtcaaagttatcacgctgaaaagcaagc | |
| | tggtgagcgacttccgtaaggattttcagttttacaaagtccgtgaaatcaacaactaccaccatgcgcacgatg | |
| | cctatctgaacgctgt (SEQ ID NO: 56) | |
| Spy-Cas9_4 | ccatgcgcacgatgcctatctgaacgctgtggtgggtaccgcgctgattaagaagtatccgaaactggaaag | Synthetic |
| | cgagttcgtgtacggtgattacaaggtttacgatgttcgtaagatgatcgcgaagtccgaacaagaaatcggca | |
| | aagcgaccgctaagtatttcttttactccaacattatgaactttttcaaaaccgagatcaccctggcaaacggtga | |
| | gatccgcaaacgtccgctgatcgagactaatggcgagactggcgaaatcgtgtgggacaaaggtcgtgactt | |
| | cgccaccgtccgtaaggtattgagcatgccgcaagtcaatattgttaagaaaaccgaagttcaaaccggtggtt | |
| | tcagcaaagagagcattctgcctaagcgcaactccgacaaactgattgcccgtaagaaggattgggacccga | |
| | aaaagtatggcggtttcgatagcccaactgtggcatacagcgtgctggtggttgccaaagtggagaaaggtaa | |
| | gtccaagaagctgaaatctgtcaaagagctgctgggcatcaccattatggagcgcagcagctttgagaaaaat | |
| | ccaatcgacttcctggaagcgaagggctacaaagaggtcaagaaagacctgatcatcaagttgccaaagtac | |
| | agcctgttcgagctggagaatggtcgtaagcgcatgctggcctctgccggtgaactgcaaaagggtaacgaa | |
| | ctggcgctgccgtcgaaatacgttaactttctgtacctggcatcccactacgagaaactgaaaggcagccctg | |
| | aagataacgagcaaaaacaactgtttgttgagcagcacaaacactatctggatgagatcattgaacagattag | |
| | cgaattcagcaagcgtgtgatcctggcggacgcgaacctggacaaagtcctgtccgcgtacaataaacatcg | |
| | cgacaaaccgattcgtgagcaggcggaaaacattatccacctgtttaccctgacgaatctgggtgcccctgcg | |
| | gcgtttaagtactttgacactactatcgatcgtaaacgttatacgagcaccaaagaggttctggatgcgaccctg | |
| | attcaccagagcattaccggcctgtatgaaacgcgtatcgacctgagccaattgggtggtgaccgctctcgtg | |
| | cagatccgaaaaagaaacgcaaagtcgatccgaagaagaagcgcaaggtggacccgaagaaaaagcgta | |
| | aagtcggctctaccggtagccgtggctctggttcgctcgagcaccaccaccaccaccactga (SEQ ID | |
| | NO: 57) | |
| Spy-Cas9_5 | ccatgcgcacgatgcctatctgaacgctgtggtgggtaccgcgctgattaagaagtatccgaaactggaaag | Synthetic |
| | cgagttcgtgtacggtgattacaaggtttacgatgttcgtaagatgatcgcgaagtccgaacaagaaatcggca | |
| | aagcgaccgctaagtatttcttttactccaacattatgaactttttcaaaaccgagatcaccctggcaaacggtga | |
| | gatccgcaaacgtccgctgatcgagactaatggcgagactggcgaaatcgtgtgggacaaaggtcgtgactt | |
| | cgccaccgtccgtaaggtattgagcatgccgcaagtcaatattgttaagaaaaccgaagttcaaaccggtggtt | |
| | tcagcaaagagagcattctgcctaagcgcaactccgacaaactgattgcccgtaagaaggattgggacccga | |
| | aaaagtatggcggtttcgatagcccaactgtggcatacagcgtgctggtggttgccaaagtggagaaaggtaa | |
| | gtccaagaagctgaaatctgtcaaagagctgctgggcatcaccattatggagcgcagcagctttgagaaaaat | |
| | ccaatcgacttcctggaagcgaagggctacaaagaggtcaagaaagacctgatcatcaagttgccaaagtac | |
| | agcctgttcgagctggagaatggtcgtaagcgcatgctggcctctgccggtgaactgcaaaagggtaacgaa | |
| | ctggcgctgccgtcgaaatacgttaactttctgtacctggcatcccactacgagaaactgaaaggcagccctg | |
| | aagataacgagcaaaaacaactgtttgttgagcagcacaaacactatctggatgagatcattgaacagattag | |
| | cgaattcagcaagcgtgtgatcctggcggacgcgaacctggacaaagtcctgtccgcgtacaataaacatcg | |
| | cgacaaaccgattcgtgagcaggcggaaaacattatccacctgtttaccctgacgaatctgggtgcccctgcg | |
| | gcgtttaagtactttgacactactatcgatcgtaaacgttatacgagcaccaaagaggttctggatgcgaccctg | |
| | attcaccagagcattaccggcctgtatgaaacgcgtatcgacctgagccaattgggtggtgaccgctctcgtg | |
| | cagatccgaaaaagaaacgcaaagtcgatccgaagaagaagcgcaaggtggacccgaagaaaaagcgta | |
| | aagtcggctctaccggtagccgtggctctggttcgTAActcgagcaccaccaccaccaccactga | |
| | (SEQ ID NO: 58) | |
| AAVS1_ | TAATACGACTCACTATAGGGCTACTGGCCTTATCTCACGTTTTAGA | PCR |
| T23826 | GCTAGAAATAGCAAGTTAAAATAAGGCTAGTCCGTTATCAACTTG | |
| | AAAAAGTGGCACCGAGTCGGTGCTTTT (SEQ ID NO: 59) | |
| V5C2-L- | gcggataacaattcccctctagaatagaaggagatttaaatgagccgtaaagaagcacgcgagctctgttacc | Synthetic |
| HRPC2 | cggagaatggtctggaagcactgattagatctggaggtggaggttcaggtggaggtggatccggtggtggag | |
| | gatcatattatctgcgtaaacgtattctgtgctacccggaaaatcaggttctggaacgtagcaatgaaggtagtg | |
| | gtagcaagcttctcgagcaccaccaccaccaccactga (SEQ ID NO: 60) | |
| AAVS1 | ggaggaatatgtcccagatagcactggggactctttaaggaaagaaggatggagaaagagaaagggagta | PCR |
| | gaggcggccacgacctggtgaacacctaggacgcaccattctcacaaagggagttttccacacggacaccc | |
| | ccctcctcaccacagccctgccaggacggggctggctactggccttatctcacaggtaaaactgacgcac | |
| | ggaggaacaatataaattggggactagaaaggtgaagagccaaagttagaactcaggaccaacttattctgat | |
| | tttgtttttccaaactgcttctcctcttgggaagtgtaaggaagctgcagcaccaggatcagtgaaacgcaccag | |
| | acggccgcgtcagagcagctcaggttctgggagagggtagcgcagggtggccactgagaaccgggcagg | |
| | tcacgcatcccccccttccctcccaccccctgccaagctctccctcccaggatcctctctggctccatcgtaag | |
| | caaacctt (SEQ ID NO: 61) | |

TABLE 4

| | | | Complementary amino | | |
|---|---|---|---|---|---|
| | Inter- | PDB | acid pairing (CAAP, | Pairing | |
| Protein (chain_structure) | action | ID | underlined) Box | Orientation | Source |
| Amyloid Precursor E2 (chain | Homo | 3NYL | KAKERLEA (SEQ ID | Antiparallel | Homo |
| A_helix 2) | dimer | | NO: 62) | | sapiens |
| Amyloid Precursor E2 (chain | | | FHKLTHQR (SEQ ID | | |
| B_helix 4) | | | NO: 63) | | |
| Amyloid Precursor E2 (chain | Homo | 3NYL | ERQQLVET (SEQ ID | Antiparallel | Homo |
| A_helix 3) | dimer | | NO: 64) | | sapiens |
| Amyloid Precursor E2 (chain | | | LSLSQNMR (SEQ ID | | |
| B_helix 5) | | | NO: 65) | | |
| APPL1-BAR (chain A_helix 2) | Homo | 2Z0N | ELSAATHL (SEQ ID | Antiparallel | Homo |
| APPL1-BAR (chain B_helix 2) | dimer | | NO: 66) | | sapiens |
| | | | LHTAASLE (SEQ ID | | |
| | | | NO: 67) | | |
| APPL1-BAR (chain A_helix 7) | Homo | 2Z0N | TSVQNVRR (SEQ ID | Antiparallel | Homo |
| APPL1-BAR (chain B_helix 5) | dimer | | NO: 68) | | sapiens |
| | | | RSTYVDET (SEQ ID | | |
| | | | NO: 69) | | |
| C.esp1396i (chain A_helix 4) | Homo | 3G5G | FEMLIKEILK (SEQ | Antiparallel | Enterobacter |
| C.esp1396i (chain B_helix 4) | dimer | | ID NO: 70) | | sp. RFL1396 |
| | | | KLIEKILMEF (SEQ | | |
| | | | ID NO: 71) | | |
| Cagl (chain A_helix 2) | Homo | 4CII | IGGTASLITASQ | Antiparallel | Helicobacter |
| Cagl (chain B_helix 2) | dimer | | (SEQ ID NO: 72) | | pylori 26695 |
| | | | YQRKSQELSREL | | |
| | | | (SEQ ID NO: 73) | | |
| Cagl (chain A_helix 2) | Homo | 4CII | LEELDALERSLEQS | Antiparallel | Helicobacter |
| Cagl (chain B_helix 2) | dimer | | KR | | pylori 26695 |
| | | | (SEQ ID NO: 74) | | |
| | | | KLSEVLTQSATILSA | | |
| | | | T | | |
| | | | (SEQ ID NO: 75) | | |
| Cce_0567 (chain A_helix 1) | Homo | 3CSX | LKKKVRKL (SEQ ID | Antiparallel | Cyanobacterium |
| Cce_0567 (chain B_helix 1) | dimer | | NO: 76) | | Cyanothece |
| | | | KKKLQDLE (SEQ ID | | |
| | | | NO: 77) | | |
| Csor (chain A_helix 2) | Homo | 2HH7 | QSSLERAN (SEQ ID | Antiparallel | Mycobacterium |
| Csor (chain B_helix 2) | dimer | | NO: 78) | | tuberculosis |
| | | | NARELSSQ (SEQ ID | | |
| | | | NO: 79) | | |
| Cytochrome C (chain A_helix 1) | Homo | 1BBH | AGLSPEEQ (SEQ ID | Antiparallel | Allochromatium |
| Cytochrome C (chain B_helix 1) | dimer | | NO: 80) | | vinosum |
| | | | GAQRTEIQ (SEQ ID | | |
| | | | NO: 81) | | |
| Cytochrome C (chain A_helix 2) | Homo | 1BBH | IAAIANSG (SEQ ID | Antiparallel | Allochromatium |
| Cytochrome C (chain B_helix 2) | dimer | | NO: 82) | | vinosum |
| | | | MGSNAIAA (SEQ ID | | |
| | | | NO: 83) | | |
| DD_Ribeta_PKA (chain | Homo | 4F9K | LREHFEKLEK (SEQ | Antiparallel | Homo |
| A_he1ix3) | dimer | | ID NO: 84) | | sapiens |
| DD_Ribeta_PKA (chain | | | KELKEFHERL (SEQ | | |
| B_he1ix3) | | | ID NO: 85) | | |
| Endothelin-1 (chain A_beta sheet) | Homo | 1T7H | KRCSCSSL (SEQ ID | Antiparallel | Homo |
| Endothelin-1 (chain B_beta sheet) | dimer | | NO: 86) | | sapiens |
| | | | LSSCSCRK (SEQ ID | | |
| | | | NO: 87) | | |
| Fkbp22 (chain A_helix 1) | Homo | 3B09 | SYGVGRQG (SEQ ID | Antiparallel | Shewanella |
| Fkbp22 (chain B_helix 3) | dimer | | NO: 88) | | sp. SIB1 |
| | | | RRSIETFA (SEQ ID | | |
| | | | NO: 89) | | |
| Gp7-Myh7-EB1 (chain A_helix 3) | Homo | 4XA1 | LEKEKSEFKLEL | Antiparallel | Homo |
| Gp7-Myh7-EB1 (chain B_helix 3) | dimer | | (SEQ ID NO: 90) | | sapiens |
| | | | KLEKEKSEFKLE | | |
| | | | (SEQ ID NO: 91) | | |
| HDAg (chain A_helix 1) | Homo | 1A92 | KLEELERDLRKL | Antiparallel | Hepatitis |
| HDAg (chain B_helix 1) | octamer | | (SEQ ID NO: 92) | | delta virus |
| | | | LKRLDRELEELK | | |
| | | | (SEQ ID NO: 93) | | |
| Hi0947 (chain A_helix 2) | Homo | 2JUZ | ASNLLTTS (SEQ ID | Antiparallel | Haemophilus |
| Hi0947 (chain B_helix 2) | dimer | | NO: 94) | | influenzae |
| | | | STTLLNSA (SEQ ID | | |
| | | | NO: 95) | | |
| Hi0947 (chain A_helix 3) | Homo | 2JUZ | SLINAVKT (SEQ ID | Antiparallel | Haemophilus |
| Hi0947 (chain B_helix 3) | dimer | | NO: 96) | | influenzae |
| | | | TKVANILS (SEQ ID | | |
| | | | NO: 97) | | |
| Hp0062 (chain A_helix 1) | Homo | 3FX7 | LERFKELL (SEQ ID | Antiparallel | Helicobacter |
| Hp0062 (chain B_helix 1) | dimer | | NO: 98) | | pylori |
| | | | RLLEKFRE (SEQ ID | | |
| | | | NO: 99) | | |
| Hp0062 (chain A_helix 2) | Homo | 3FX7 | DKFSEVLDNLKSTF | Antiparallel | Helicobacter |
| Hp0062 (chain B_helix 2) | dimer | | NEFDEAAQEQIAWL | | pylori |
| | | | KERI (SEQ ID | | |
| | | | NO: 100) | | |
| | | | IREKLWAIQEQAAE | | |
| | | | DFENFTSKLNDLVE | | |
| | | | SFKD (SEQ ID | | |
| | | | NO: 101) | | |
| If1 (chain A_helix 1) | Homo | 1GMJ | QSIKKLKQS (SEQ ID | Antiparallel | Bos taurus |
| If1 (chain B_helix 1) | dimer | | NO: 102) | | |
| | | | LAALQEKAR (SEQ | | |
| | | | ID NO: 103) | | |
| Jip3 (chain A_helix 1) | Homo | 4PXJ | LSGEQEVLRGELEA | Antiparallel | Homo |
| Jip3 (chain B_helix 1) | dimer | | AK | | sapiens |
| | | | (SEQ ID NO: 104) | | |
| | | | KAAELEGRLVEQE | | |
| | | | GSL | | |
| | | | (SEQ ID NO: 105) | | |
| Lambda CRO Repressor (chain | Homo | 1D1L | MEQRITLK (SEQ ID | Antiparallel | Bacteriophage |
| A_beta sheet 1) | dimer | | NO: 106) | | Lambda |
| Lambda CRO Repressor (chain | | | DKLTIRQE (SEQ ID | | |
| B_beta sheet 1) | | | NO: 107) | | |
| Rev (chain A_helix 1) | Homo | 3LPH | RLIKFLYQS (SEQ ID | Antiparallel | HIV type 1 |
| Rev (chain B_helix 1) | dimer | | NO: 108) | | (HXB3 |
| | | | SQYLFKILR (SEQ ID | | ISOLATE) |
| | | | NO: 109) | | |
| Rev (chain A_helix 2) | Homo | 3LPH | SERIRSTYLGR (SEQ | Antiparallel | HIV type 1 |
| Rev (chain B_helix 2) | dimer | | ID NO: 110) | | (HXB3 |
| | | | RGLYTSRIRES (SEQ | | ISOLATE) |
| | | | ID NO: 111) | | |
| ROM (chain A_helix 1) | Homo | 2IJK | FIRSQTLT (SEQ ID | Antiparallel | Escherichia |
| ROM (chain B_helix 1) | dimer | | NO: 112) | | coli |
| | | | ELLTLTQS (SEQ ID | | |
| | | | NO: 113) | | |
| ROM (chain A_helix 2) | Homo | 2IJK | ESLHDHADEL (SEQ | Antiparallel | Escherichia |
| ROM (chain B_helix 2) | dimer | | ID NO: 114) | | coli |
| | | | FRALCSRYLE (SEQ | | |
| | | | ID NO: 115) | | |
| Trim25 (chain A_helix1) | Homo | 4LTB | SLSQASADL (SEQ ID | Antiparallel | Homo |
| Trim25 (chain B_helix1) | dimer | | NO: 116) | | sapiens |
| | | | RKTLSQEIE (SEQ ID | | |
| | | | NO: 117) | | |
| Trim25 (chain A_he1ix3) | Homo | 4LTB | QSTIDLKN (SEQ ID | Antiparallel | Homo |
| Trim25 (chain B_he1ix3) | dimer | | NO: 118) | | sapiens |
| | | | LRGICQKL (SEQ ID | | |
| | | | NO: 119) | | |
| Usp8 (chain A_helix 1) | Homo | 2A9U | KSYVHSALKIFKTA | Antiparallel | Homo |
| Usp8 (chain B_helix 1) | dimer | | EECRL | | sapiens |
| | | | (SEQ ID NO: 120) | | |
| | | | LRCEEATKFIKLAS | | |
| | | | HVYSK | | |
| | | | (SEQ ID NO: 121) | | |
| Usp8 (chain A_helix 2) | Homo | 2A9U | YVLYMKYV (SEQ ID | Antiparallel | Homo |
| Usp8 (chain B_helix 2) | dimer | | NO: 122) | | sapiens |
| | | | VYKMYLVY (SEQ ID | | |
| | | | NO: 123) | | |
| Xcl1 (chain A_beta sheet 3) | Homo | 2N54 | RCVIFITF (SEQ ID | Antiparallel | Homo |
| Xcl1 (chain B_beta sheet 2) | dimer | | NO: 124) | | sapiens |
| | | | ITYTKIRS (SEQ ID | | |
| | | | NO: 125) | | |
| Gemin6 (chain A_beta sheet 5) | Hetero | 1Y96 | GSMSVTGI (SEQ ID | Antiparallel | Homo |
| Gemin7 (chain B_beta sheet 7) | dimer | | NO: 126) | | sapiens |
| | | | PKFTYSII (SEQ ID | | |
| | | | NO: 127) | | |
| Lin-7 (chain A_helix 1) | Hetero | 1ZL8 | QRILELMEHV (SEQ | Antiparallel | Caenorhabditis |
| Lin-2 (chain B_helix 2) | dimer | | ID NO: 128) | | elegans |
| | | | LIRKLEKADN (SEQ | | Homo |
| | | | ID NO: 129) | | sapiens |
| Lin-7 (chain A_helix 2) | Hetero | 1ZL8 | ASLQQVLQ (SEQ ID | Antiparallel | Caenorhabditis |
| Lin-2 (chain B_helix 1) | dimer | | NO: 130) | | elegans |
| | | | SIEELVEK (SEQ ID | | Homo |
| | | | NO: 131) | | sapiens |
| Med7 (chain A_helix 1) | Hetero | lYKH | IQELRKLL (SEQ ID | Antiparallel | Saccharomyces |
| Srb7 (chain B_helix 2) | dimer | | NO: 132) | | cerevisiae |
| | | | DILKNIQR (SEQ ID | | |
| | | | NO: 133) | | |
| Mst1 (chain A_helix) | Hetero | 40H8 | LQKRLLALDP (SEQ | Antiparallel | Homo |
| Rassf5 Sarah (chain B_helix) | dimer | | ID NO: 134) | | sapiens |
| | | | ERLAEELKQR (SEQ | | |
| | | | ID NO: 135) | | |
| PALS-1-L27N (chain A_helix 1) | Hetero | 1VF6 | VLDRLKMK (SEQ ID | Antiparallel | Homo |
| PATJ-L27 (chain B_helix 2) | dimer | | NO: 136) | | sapiens |
| | | | NQVLQLLL (SEQ ID | | Mus |
| | | | NO: 137) | | musculus |
| PALS-1-L27N (chain A_helix 2) | Hetero | 1VF6 | LSMFYETL (SEQ ID | Antiparallel | Homo |
| PATJ-L27 (chain B_helix 1) | dimer | | NO: 138) | | sapiens |
| | | | QIHKLSSF (SEQ ID | | Mus |
| | | | NO: 139) | | musculus |
| TAF(II)-18 (chain A_helix 1) | Hetero | 1BH8 | LFSKELRC (SEQ ID | Antiparallel | Homo |
| TAF(II)-28 (chain B_helix 1) | dimer | | NO: 140) | | sapiens |
| | | | EYRNLQEE (SEQ ID | | |
| | | | NO: 141) | | |
| TAF(II)-18 (chain A_helix 2) | Hetero | 1BH8 | LEDLVIEFITEMTH | Antiparallel | Homo |
| TAF(II)-28 (chain B_helix 3) | dimer | | (SEQ ID NO: 142) | | sapiens |
| | | | EVVEGVFVKSIGSM | | |
| | | | (SEQ ID NO: 143) | | |
| Type I Antifreeze Protein (chain | Homo | 4KE2 | No CAAP Box | Antiparallel | Pseudopleuro |
| A_helix) | dimer | | | | nectes |
| Type I Antifreeze Protein (chain | | | | | americanus |
| B_helix) | | | | | |
| Swi5 (chain B_helix) | Homo | 3VIR | VQKHIDLLHTYNEI | Antiparallel | Schizosaccharomyces |
| Swi5(chain A_helix) | tetramer | | (SEQ ID NO: 144) | | pombe |
| | | | HLLDIHKQVTQKA | | |
| | | | D | | |
| | | | (SEQ ID NO: 145) | | |
| Swi5 (chain C_helix) | Homo | 3VIR | EQQKEQLESSLQ | Antiparallel | Schizosaccharomyces |
| Swi5(chain A_helix) | tetramer | | (SEQ ID NO: 146) | | pombe |
| | | | LKALADQLSSEL | | |
| | | | (SEQ ID NO: 147) | | |
| Arenicin-2 (chain A_beta sheet 1) | Homo | 2L8X | VYAYVRIR (SEQ ID | Parallel | Arenicola |
| Arenicin-2 (chain B_beta sheet 1) | dimer | | NO: 148) | | marina |
| | | | RWCVYAYV (SEQ ID | | (lugworm) |
| | | | NO: 149) | | |
| Beta-myosin S2 (chain A_helix 1) | Homo | 2FXO | EALEKSEARRKELE | Parallel | Homo |
| Beta-myosin S2 (chain B_helix 1) | dimer | | E | | sapiens |
| | | | (SEQ ID NO: 150) | | |
| | | | LKEALEKSEARRKE | | |
| | | | L | | |
| | | | (SEQ ID NO: 151) | | |
| Beta-myosin S2 (chain A_helix 2) | Homo | 2FXO | EKNDLQLQVQ (SEQ | Parallel | Homo |
| Beta-myosin S2 (chain B_helix 2) | dimer | | ID NO: 152) | | sapiens |
| | | | LLQEKNDLQL (SEQ | | |
| | | | ID NO: 153) | | |
| Beta-myosin S2 (chain A_helix 3) | Homo | 2FXO | ELKRDIDDLE (SEQ | Parallel | Homo |
| Beta-myosin S2 (chain B_helix 3) | dimer | | ID NO: 154) | | sapiens |
| | | | LKRDIDDLEL (SEQ | | |
| | | | ID NO: 155) | | |
| Cc1-fha (chain A_helix 1) | Homo | 5DJO | LKEKLEES (SEQ ID | Parallel | Mus |
| Cc1-fha (chain B_helix 1) | dimer | | NO: 156) | | musculus |
| | | | ELKEKLEE (SEQ ID | | |
| | | | NO: 157) | | |
| Cc2-LZ (chain A_helix 1) | Homo | 4BWN | LEDLKQQLQ (SEQ | Parallel | Homo |
| Cc2-LZ (chain B_helix 1) | dimer | | ID NO: 158) | | sapiens |
| | | | QLEDLKQQL (SEQ | | |
| | | | ID NO: 159) | | |
| Cc2-LZ (chain A_helix 2) | Homo | 4BWN | LLQEQLEQLQ (SEQ | Parallel | Homo |
| Cc2-LZ (chain B_helix 2) | dimer | | ID NO: 160) | | sapiens |
| | | | ELLQEQLEQL (SEQ | | |
| | | | ID NO: 161) | | |
| Cenp-b (chain A_helix 1) | Homo | 1UFI | AYFAMVKR (SEQ ID | Parallel | Homo |
| Cenp-b (chain B_helix 1) | dimer | | NO: 162) | | sapiens |
| | | | GEAMAYFA (SEQ ID | | |
| | | | NO: 163) | | |
| Cenp-b (chain A_helix 2) | Homo | 1UFI | HLEHDLVH (SEQ ID | Parallel | Homo |
| Cenp-b (chain B_helix 2) | dimer | | NO: 164) | | sapiens |
| | | | VQSHILHL (SEQ ID | | |
| | | | NO: 165) | | |
| cGMP-dependent protein kinase | Homo | 1ZXA | LEKRLSEK (SEQ ID | Parallel | Homo |
| (chain A_helix) | dimer | | NO: 166) | | sapiens |
| cGMP-dependent protein kinase | | | KELEKRLS (SEQ ID | | |
| (chain B_helix) | | | NO: 167) | | |
| DSX (chain A_helix 3) | Homo | 1ZV1 | EEGQYVVNEYSR | Parallel | Drosophila |
| DSX (chain B_helix 2) | dimer | | (SEQ ID NO: 168) | | melanogaster |
| | | | LMPLMYVILKDA | | |
| | | | (SEQ ID NO: 169) | | |
| Ferritin (chain A_helix 1) | Homo | 1LB3 | VEAAVNRL (SEQ ID | Parallel | Mus |
| Ferritin (chain B_helix 2) | 24 mer | | NO: 170) | | musculus |
| | | | HFFRELAE (SEQ ID | | |
| | | | NO: 171) | | |
| FGFR3 (chain A_helix 1) | Homo | 2LZL | AGSVYAGI (SEQ ID | Parallel | Homo |
| FGFR3 (chain B_helix 1) | dimer | | NO: 172) | | sapiens |
| | | | EAGSVYAG (SEQ ID | | |
| | | | NO: 173) | | |
| Fkbp22 (chain A_helix 1) | Homo | 3B09 | GVGRQGEQ (SEQ ID | Parallel | Shewanella |
| Fkbp22 (chain B_helix 2) | dimer | | NO: 174) | | sp. SIB1 |
| | | | AGLADAFA (SEQ ID | | |
| | | | NO: 175) | | |
| Gal4 (chain A_helix 1) | Homo | 1HBW | RLERLEQL (SEQ ID | Parallel | Saccharomyces |
| Gal4 (chain B_helix 1) | dimer | | NO: 176) | | cerevisiae |
| | | | SRLERLEQ (SEQ ID | | |
| | | | NO: 177) | | |
| GCN4 (chain A_helix 2) | Homo | 2DGC | RRSRARKLQRMKQ | Parallel | Saccharomyces |
| GCN4 (chain B_helix 2) | dimer | | LE | | cerevisiae |
| | | | (SEQ ID NO: 178) | | |
| | | | ARRSRARKLQRMK | | |
| | | | QL | | |
| | | | (SEQ ID NO: 179) | | |
| Gld1 (chain A_helix 1) | Homo | 3K6T | ADLVKEKK (SEQ ID | Parallel | Caenorhabditis |
| Gld1 (chain B_helix 2) | dimer | | NO: 180) | | elegans |
| | | | NVERLLDD (SEQ ID | | |
| | | | NO: 181) | | |
| Gld1 (chain A_helix 2) | Homo | 3K6T | SNVERLLD (SEQ ID | Parallel | Caenorhabditis |
| Gld1 (chain B_helix 1) | dimer | | NO: 182) | | elegans |
| | | | LADLVKEK (SEQ ID | | |
| | | | NO: 183) | | |
| Hmfa (chain A_helix 2) | Homo | 1HTA | SDDARIAL (SEQ ID | Parallel | Methanobacterium |
| Hmfa (chain B_helix 1) | dimer | | NO: 184) | | fervidus |
| | | | RIIKNAGA (SEQ ID | | |
| | | | NO: 185) | | |
| Hnf-1alpha (chain A_helix 1) | Homo | 1JB6 | LSQLQTEL (SEQ ID | Parallel | Mus |
| Hnf-1alpha (chain B_helix 1) | dimer | | NO: 186) | | musculus |
| | | | KLSQLQTE (SEQ ID | | |
| | | | NO: 187) | | |
| Hnf-1alpha (chain A_helix 1) | Homo | 1JB6 | LSQLQTEL (SEQ ID | Parallel | Mus |
| Hnf-1alpha (chain B_helix 2) | dimer | | NO: 188) | | musculus |
| | | | EALIQALG (SEQ ID | | |
| | | | NO: 189) | | |
| Hv1 (chain A_helix 1) | Homo | 3VMX | LNKLLKQN (SEQ ID | Parallel | Mus |
| Hv1 (chain B_helix 1) | dimer | | NO: 190) | | musculus |
| | | | ERLNKLLK (SEQ ID | | |
| | | | NO: 191) | | |
| Hy5 (chain A_helix) | Homo | 20QQ | SAYLSELE (SEQ ID | Parallel | Arabidopsis |
| Hy5 (chain B_helix) | dimer | | NO: 192) | | thaliana |
| | | | GSAYLSEL (SEQ ID | | |
| | | | NO: 193) | | |
| Interleukin-10 (chain A_helix 4) | Homo | 1ILK | ALSEMIQF (SEQ ID | Parallel | Homo |
| Interleukin-10 (chain B_helix 6) | dimer | | NO: 194) | | sapiens |
| | | | SKAVEQVK (SEQ ID | | |
| | | | NO: 195) | | |
| Lamin Coil 2B (chain A_helix 1) | Homo | 1X8Y | LARERDTSRRLLAE | Parallel | Homo |
| Lamin Coil 2B (chain B_helix 1) | dimer | | KEREMA | | sapiens |
| | | | (SEQ ID NO: 196) | | |
| | | | EDSLARERDTSRRL | | |
| | | | LAEKER | | |
| | | | (SEQ ID NO: 197) | | |
| Max (chain A_helix 1) | Homo | 1R05 | DSFHSLRD (SEQ ID | Parallel | Homo |
| Max (chain B_helix 1) | dimer | | NO: 198) | | sapiens |
| | | | IQYMRRKV (SEQ ID | | |
| | | | NO: 199) | | |
| Max (chain A_helix 1) | Homo | 1R05 | RALEGSGC (SEQ ID | Parallel | Homo |
| Max (chain B_helix 1) | dimer | | NO: 200) | | sapiens |
| | | | VRALEGSG (SEQ ID | | |
| | | | NO: 201) | | |
| Myosin X (chain A_helix 2) | Homo | 5HMO | KQVEEILR (SEQ ID | Parallel | Bos taurus |
| Myosin X (chain C_helix 3) | dimer | | NO: 202) | | |
| | | | LQQLRDEE (SEQ ID | | |
| | | | NO: 203) | | |
| Myosin X (chain A_helix 3) | Homo | 5HMO | LQKLQQLRD (SEQ | Parallel | Bos taurus |
| Myosin X (chain C_helix 2) | dimer | | ID NO: 204) | | |
| | | | EILRLEKEI (SEQ ID | | |
| | | | NO: 205) | | |
| NEMO(chain A_helix 1) | Homo | 4OWF | LRQQLQQA (SEQ ID | Parallel | Mus |
| NEMO (chain B_helix 1) | dimer | | NO: 206) | | musculus |
| | | | EDLRQQLQ (SEQ ID | | |
| | | | NO: 207) | | |
| NEMO(chain A_helix 3) | Homo | 4OWF | QEQLEQLQREF | Parallel | Mus |
| NEMO (chain B_helix 3) | dimer | | (SEQ ID NO: 208) | | musculus |
| | | | LQEQLEQLQRE | | |
| | | | (SEQ ID NO: 209) | | |
| Nsp3 (chain A_helix 1) | Homo | 1LJ2 | LQVYNNKLE (SEQ | Parallel | Simian |
| Nsp3 (chain B_helix 3) | dimer | | ID NO: 210) | | rotavirus |
| | | | ELQVYNNKL (SEQ | | A/SA11 |
| | | | ID NO: 211) | | |
| Nsp3 (chain A_helix 1) | Homo | 1LJ2 | NKIGSLTS (SEQ ID | Parallel | Simian |
| Nsp3 (chain B_helix 3) | dimer | | NO: 212) | | rotavirus |
| | | | AFDDLESV (SEQ ID | | A/SA12 |
| | | | NO: 213) | | |
| p53LZ2 (chain A_helix) | Homo | 4OWI | ELEVARLKKL (SEQ | Parallel | Synthetic |
| p53LZ2 (chain B_helix) | dimer | | ID NO: 214) | | construct |
| | | | LELEVARLKK (SEQ | | |
| | | | ID NO: 215) | | |
| Pkg1-Alpha (chain A_helix) | Homo | 4R4M | LKRKLHKLQ (SEQ | Parallel | Homo |
| Pkg1-Alpha (chain B_helix) | dimer | | ID NO: 216) | | sapiens |
| | | | ELKRKLHKL (SEQ | | |
| | | | ID NO: 217) | | |
| Pkg1-Beta (chain A_helix) | Homo | 3NMD | DELELELDQKDELI | Parallel | Homo |
| Pkg1-Beta (chain B_helix) | dimer | | QLQNEL | | sapiens |
| | | | (SEQ ID NO: 218) | | |
| | | | IDELELELDQKDELI | | |
| | | | QLQNE | | |
| | | | (SEQ ID NO: 219) | | |
| Put3 (chain A_helix) | Homo | 1AJY | LQQLQKDL (SEQ ID | Parallel | Saccharomyces |
| Put3 (chain B_helix) | dimer | | NO: 220) | | cerevisiae |
| | | | KYLQQLQK (SEQ ID | | |
| | | | NO: 221) | | |
| Qua1 (chain A_helix 2) | Homo | 4DNN | LDEEISRVRKD (SEQ | Parallel | Mus |
| Qua1 (chain B_helix 2) | dimer | | ID NO: 222) | | musculus |
| | | | ERLLDEEISRV (SEQ | | |
| | | | ID NO: 223) | | |
| Sgt2 (chain A_helix 2) | Homo | 3ZDM | GADSLNVAMDCISE | Parallel | Saccharomyces |
| Sgt2 (chain B_helix 1) | tetramer | | A | | cerevisiae |
| | | | (SEQ ID NO: 224) | | |
| | | | ASKEEIAALIVNYFS | | |
| | | | (SEQ ID NO: 225) | | |
| TarH (chain A_helix 1) | Homo | 1VLT | LRQQSEL (SEQ ID | Parallel | Salmonella |
| TarH (chain B_helix 1) | dimer | | NO: 226) | | enterica |
| | | | ISNELRQQ (SEQ ID | | serovar |
| | | | NO: 227) | | Typhimurium |
| Ylan (chain A_helix 1) | Homo | 20DM | EVLDTQFGLQKEVD | Parallel | Staphylococcus |
| Ylan (chain B_helix 1) | dimer | | FAVK | | aureus |
| | | | (SEQ ID NO: 228) | | subsp. aureus |
| | | | LYEEVLDTQFGLQK | | MW2 |
| | | | EVDF | | |
| | | | (SEQ ID NO: 229) | | |
| AMSH (chain B_helix 1) | Hetero | 2XZE | KAEELKAE (SEQ ID | Parallel | Homo |
| CHAMP3 (chain R_helix 1) | dimer | | NO: 230) | | sapiens |
| | | | SRLATLRS (SEQ ID | | |
| | | | NO: 231) | | |
| ATF4 (chain A_helix 1) | Hetero | 1CI6 | LEKKNEALKERA | Parallel | Mus |
| C/EBP beta (chain B_helix 1) | dimer | | (SEQ ID NO: 232) | | musculus |
| | | | ERLQKKVEQLSR | | |
| | | | (SEQ ID NO: 233) | | |
| c-Fos (chain A_helix 1) | Hetero | 2WT7 | LEDEKSALQ (SEQ | Parallel | Mus |
| MafB (chain B_helix 1) | dimer | | ID NO: 234) | | musculus |
| | | | QLIQQVEQL (SEQ | | |
| | | | ID NO: 235) | | |
| c-Jun (chain F_helix 2) | Hetero | 1FOS | LKAQNSEL (SEQ ID | Parallel | Homo |
| c-Fos (chain E_helix 2) | dimer | | NO: 236) | | sapiens |
| | | | EDEKSALQ (SEQ ID | | |
| | | | NO: 237) | | |
| c-Jun (chain F_helix 2) | Hetero | 1FOS | VAQLKQKV (SEQ ID | Parallel | Homo |
| c-Fos (chain E_helix 2) | dimer | | NO: 238) | | sapiens |
| | | | EKLEFILA (SEQ ID | | |
| | | | NO: 239) | | |
| DP1 (chain A_helix 1) | Hetero | 2AZE | AQECQNLE (SEQ ID | Parallel | Homo |
| E2F1 (chain B_helix 1) | dimer | | NO: 240) | | sapiens |
| | | | RLEGLTQD (SEQ ID | | |
| | | | NO: 241) | | |
| E47 (chain A_helix 1) | Hetero | 2QL2 | LILQQAVQVI (SEQ | Parallel | Mus |
| NeuroD1 (chain B_helix 1) | dimer | | ID NO: 242) | | musculus |
| | | | KIETLRLAKN (SEQ | | |
| | | | ID NO: 243) | | |
| ErbB2 (chain A_loop 1) | Hetero | 2KS1 | GCPAEQRA (SEQ ID | Parallel | Homo |
| ErbB1(chain B_loop 1) | dimer | | NO: 244) | | sapiens |
| | | | TNGPKIPS (SEQ ID | | |
| | | | NO: 245) | | |
| GBR1 (chain A_helix 1) | Hetero | 4PAS | EERVSELRHQLQ | Parallel | Homo |
| GBR2 (chain B_helix 1) | dimer | | (SEQ ID NO: 246) | | sapiens |
| | | | LDKDLEEVTMQL | | |
| | | | (SEQ ID NO: 247) | | |
| Lin-7 (chain A_helix 3) | Hetero | 1ZL8 | REVYETVY (SEQ ID | Parallel | Caenorhabditis |
| Lin-2 (chain B_helix 3) | dimer | | NO: 248) | | elegans |
| | | | THDVVAHE (SEQ ID | | Homo |
| | | | NO: 249) | | sapiens |
| Med7 (chain A_helix 3) | Hetero | 1YKH | LLEEQLEY (SEQ ID | Parallel | Saccharomyces |
| Srb7 (chain B_helix 3) | dimer | | NO: 250) | | cerevisiae |
| | | | QKKLVEVE (SEQ ID | | |
| | | | NO: 251) | | |
| Myc (chain A_helix 1) | Hetero | 1NKP | LRKRREQL (SEQ ID | Parallel | Homo |
| Max (chain B_helix 1) | dimer | | NO: 252) | | sapiens |
| | | | KRQNALLE (SEQ ID | | |
| | | | NO: 253) | | |
| SCL (chain A_helix 2) | Hetero | 2YPB | LSKNEILR (SEQ ID | Parallel | Homo |
| E47 (chain B_helix 2) | dimer | | NO: 254) | | sapiens |
| | | | KLLILQQA (SEQ ID | | |
| | | | NO: 255) | | |
| Ala-14 (chain A_helix) | Homo | 1JCD | ARANQRAD (SEQ ID | Parallel | Escherichia |
| Ala-14 (chain B_helix) | trimer | | NO: 256) | | coli |
| | | | AARANQRA (SEQ ID | | |
| | | | NO: 257) | | |
| C/EBP (chain A_helix 1) | Homo | 1NWQ | VLELTSDN (SEQ ID | Parallel | Rattus |
| C/EBP (chain B_helix 1) | dimer | | NO: 258) | | norvegicus |
| | | | KVLELTSD (SEQ ID | | |
| | | | NO: 259) | | |
| C/EBP (chain A_helix 2) | Homo | 1NWQ | QLSRELDT (SEQ ID | Parallel | Rattus |
| C/EBP (chain B_helix 2) | dimer | | NO: 260) | | norvegicus |
| | | | EQLSRELD (SEQ ID | | |
| | | | NO: 261) | | |
| c-Jun (chain A_helix) | Homo | 1JUN | KAQNSELAST (SEQ | Parallel | Homo |
| c-Jun (chain B_helix) | dimer | | ID NO: 262) | | sapiens |
| | | | LKAQNSELAS (SEQ | | |
| | | | ID NO: 263) | | |
| EB1 (chain A_helix 1) | Homo | 3GJO | KLTVEDLE (SEQ ID | Parallel | Homo |
| EB1 (chain B_helix 1) | dimer | | NO: 264) | | sapiens |
| | | | LKLTVEDL (SEQ ID | | |
| | | | NO: 265) | | |
| EB1 (chain A_helix 2) | Homo | 3GJO | LQRIVDIL (SEQ ID | Parallel | Homo |
| EB1 (chain B_helix 2) | dimer | | NO: 266) | | sapiens |
| | | | VLQRIVDI (SEQ ID | | |
| | | | NO: 267) | | |
| Geminin (chain A_helix 1) | Homo | 1T6F | EALKENEKLHK | Parallel | Homo |
| Geminin (chain B_helix 1) | dimer | | (SEQ ID NO: 268) | | sapiens |
| | | | LYEALKENEKL | | |
| | | | (SEQ ID NO: 269) | | |
| Phe-14 (chain A_helix) | Homo | 2GUV | KDDFARFNQR (SEQ | Parallel | Escherichia |
| Phe-14 (chain B_helix) | pentamer | | ID NO: 270) | | coli |
| | | | FNAFRSDFQA (SEQ | | |
| | | | ID NO: 271) | | |
| VBP (chain A_helix) | Homo | 4U5T | EIRAAFLE (SEQ ID | Parallel | Homo |
| VBP (chain B_helix) | dimer | | NO: 272) | | sapiens |
| | | | LEIRAAFL (SEQ ID | | |
| | | | NO: 273) | | |

TABLE 5
Synthetic peptides used in this study

| Peptide name | Sequence |
|---|---|
| PTD 1 | ELDKAGFIKRQL |
| | (SEQ ID NO: 14) |
| PTD 2 | LEERGVKDRQLQ |
| | (SEQ ID NO: 15) |
| PTD 3 | LEILRAKDLALE |
| | (SEQ ID NO: 16) |
| PTD 4 | LEQIKIRLF |
| | (SEQ ID NO: 17) |
| PTD 5 | LSGLNEQRTQ |
| | (SEQ ID NO: 18) |
| PTD 6 | YDVDAIVPQC |
| | (SEQ ID NO: 19) |
| PTD 7 | CLTYDSHYLQ |
| | (SEQ ID NO: 20) |
| PTD 8 | LVAHVTSRKC |
| | (SEQ ID NO: 21) |
| PTD 9 | EYRLYLRALC |
| | (SEQ ID NO: 22) |
| PTD 10 | IEIVRKKPIFC |
| | (SEQ ID NO: 24) |
| PTD 11 | CEDRLQSYDLD |
| | (SEQ ID NO: 25) |
| PTD 12 | EKLYLYYLQC |
| | (SEQ ID NO: 27) |
| PTD 13 | LEQIKIRLFGSGSHHHHHH |
| | (SEQ ID NO: 28) |
| PTD 14 | LLQVDVILLCYPENLEQIKIRLFGSGSHHHHHH |
| | (SEQ ID NO: 11) |
| PTD15 | LSRAYLSYEGSGSHHHHHH |
| | (SEQ ID NO: 29) |
| PTD16 | EYRLYLRALCYPENLSRAYLSYEGSGSHHHHHH |
| | (SEQ ID NO: 30) |
| PTD17 | EDRLQSYDLDGSGSHHHHHH |
| | (SEQ ID NO: 13) |
| PTD18 | DLDYAQLRDKCYPENEDRLQSYDLDGSGSHHHHHH |
| | (SEQ ID NO: 31) |
| PTD19 | GKPIPNPLLGLDST |
| | (SEQ ID NO: 32) |
| PTD20 | GSGSHHHHHH |
| | (SEQ ID NO: 289) |
| PTD21 | ELDKAGFIKRQLC |
| | (SEQ ID NO: 33) |
| PTD22 | LLQVDVILLHHHHHHLEQIKIRLF |
| | (SEQ ID NO: 34) |
| PTD23 | CFFDSLVKQ |
| | (SEQ ID NO: 35) |

### Example 2

### Materials and Methods

Synthetic peptides were purchased from Peptide 2.0 and are listed in Table 6. Synthetic DNA fragments are listed in Table 7. E. coli strain DH10B T1 [Thermo Fisher Scientific, catalog # 12331013] was used as a cloning host. E. coli strain BL21 Star (DE3) [Thermo Fisher Scientific, catalog # C601003] was used for the production of the recombinant proteins.

TABLE 6

| Peptide (PTD) | | |
|---|---|---|
| Number | Peptide Name | Sequence (N to C) |
| PTD6 | Sp-C9_836-841 | YDVDAIVPQC |
| PTD7 | Sp-C9_CAA836-841AP | CLTYDSHYLQ |
| PTD8 | Ec-AP_159-168 | LVAHVTSRKC |
| PTD10 | Hs-PDGF-B_136-145 | IEIVRKKPIFC |
| PTD12 | Sp-C9_CAA813-821 | EKLYLYYLQC |
| PTD13 | Sp-C9_CAA813- | LEQIKIRLFGSGSHHHHHH |
| | 821APH | |
| PTD14 | Sp-C9_CAA813- | LLQVDVILLCYPENLEQIKIRLFGSGSHHHHHH |
| | 821PAPH | |
| PTD15 | Ec-AP_CAA159- | LSRAYLSYEGSGSHHHHHH |
| | 168APH | |
| PTD16 | Ec-AP_CAA159- | EYRLYLRALCYPENLSRAYLSYEGSGSHHHHHH |
| | 168PAPH | |
| PTD17 | Hs-PDGF-B_CAA136- | EDRLQSYDLDGSGSHHHHHH |
| | 145APH | |
| PTD18 | Hs-PDGF-B_CAA136- | DLDYAQLRDKCYPENEDRLQSYDLDGSGSHHHHHH |
| | 145PAPH | |
| PTD20 | 2GS6H | GSGSHHHHHH |
| PTD23 | Hs-Bace1_Helix | CFFDSLVKQ |
| PTD24 | Hs-Brca1-Brct_51-64 | LKYFLGIAC |
| PTD25 | Hs-CCA10_51-58 | NFIQLCLEC |
| PTD26 | Hs-PDGDR_109-116 | EITEITIPC |
| PTD27 | Hs-Hsp90_44-51 | FLRELISNC |
| PTD28 | Hs-EstrogenR_50-57 | LTNLADREC |
| PTD29 | Hs-Xiap_30-37 | MVQEAIRMC |
| PTD32 | Hs-Renin_115-122 | LPFMLAEFC |

TABLE 7

| Name | Sequence (5′ to 3′) |
|---|---|
| 92_6HNLS | CCCTCTAGAATAGAAGGAGATTTAAATGCACCATCACCACCATCACGAGCTCC |
| | TGCTGAGCGTTGAAGTTCAGCAGCTGTAAGGATCCGAAAAAGAAACGCAAAG |
| | TCCTCGAGCACCACCACCACCACCACTGAGATCCGGCT |
| 93_6HNLS | CCCTCTAGAATAGAAGGAGATTTAAATGCACCATCACCACCATCACGAGCTCT |
| | TAGAGCAGATTAAAATCCGTCTGTTTTAAGGATCCGAAAAAGAAACGCAAAG |
| | TCCTCGAGCACCACCACCACCACCACTGAGATCCGGCT |
| Sp-C9_813- | AGCGTTGAAGTTCAGCAGCTGTGCTATCCGGAAAACCTCGAATACCTGTTTAT |
| 821_CAA | TGAAAAATTAAGATCTGAAGCCGAAGGCAACGGCACTATAGACTTCGAGCTC |
| | CTGTTACAGGTGGATGTGATTCTGCTCAAAACCGGTGAAGTCAACAACTTAG |
| | AGCAGATTAAAATCCGTCTGTTTAGATCTGTGAAACAAAGCACTATT |
| Anti-Bace1 | CCCTCTAGAATAGAAGGAGATTTAAATGCACCATCACCACCATCACGAGCTC |
| | AAAAAAGAACGTGAACAGCTGCTGAAAACCGGTGAAGTCAACAACCTGAAAT |
| | ATGAACGTATTCAAGAGAGATCTGTG |
| Anti-Brca1 | CCCTCTAGAATAGAAGGAGATTTAAATGCACCATCACCACCATCACGAGCTC |
| | GAACTGGCCAAAGAATGTGATCGTTGCTATCCGGAAAACAGCATTGCAGAAG |
| | AAGTGAAAGAAAGATCTGTG |
| Anti-Xiap | CCCTCTAGAATAGAAGGAGATTTAAATGCACCATCACCACCATCACGAGCTCC |
| | ATTATGAACTGCGTCAGGCACATTGCTATCCGGAAAACCATGAAGATAGCCT |
| | GCTGATTCATAGATCTGTG |
| Anti-Hsp90 | CCCTCTAGAATAGAAGGAGATTTAAATGCACCATCACCACCATCACGAGCTC |
| | AAAGAAGAACTGGAACAGCGTATCTGCTATCCGGAAAACGTCAAAGATGAAC |
| | TGAGCCGTGAAAGATCTGTG |
| Anti-EstR | CCCTCTAGAATAGAAGGAGATTTAAATGCACCATCACCACCATCACGAGCTC |
| | GAAAGCCAAGAACGTAAAGCACTGTGCTATCCGGAAAACCTGTTAATTAGCG |
| | AAGTTGCCGAAAGATCTGTG |
| Anti-PDGFR | CCCTCTAGAATAGAAGGAGATTTAAATGCACCATCACCACCATCACGAGCTCC |
| | TGGATGCACTGGATCTGGATGGTAAAACCGGTGAAGTCAACAACCGTATTAG |
| | CGATCTGAGCATTCTGAGATCTGTG |
| Spy-Cas9_1 | ccctctagaatagaaggagatttaaatggataagaaatacagcattggtttggacattggtacgaatagcgttggttgggcagtcat |
| | taccgacgagtacaaggtgccgagcaagaagtttaaagtattgggtaacacggaccgtcacagcattaagaaaaacctgattggt |
| | gcactgctgtttgacagcggtgaaactgcagaggcgactcgcctgaagcgtaccgcgcgtcgccgctatactcgtcgtaaaaac |
| | cgtatctgctatctgcaggagatctttagcaacgagatggcgaaggttgatgacagcttctttcaccgtctggaagaaagcttcctg |
| | gtcgaagaggacaaaaagcacgagcgccatccgatcttcggcaacattgtggacgaagtggcttatcatgaaaagtatccgacc |
| | atttatcatctgcgtaagaagctggttgatagcaccgataaagcggatctgcgtctgatttacctggcactggcccacatgatcaag |
| | tttcgcggccactttctgatcgagggtgatctgaatccggacaatagcgacgttgacaagctgttcatccaactggtccaaacgtac |
| | aaccagctgttcgaagaaaacccgatcaacgcgagcggtgtggatgcaaaagctattctgagcgcgcgtctgagcaagagccg |
| | tcgtttggagaatctgatcgcgcaattgccgggtgagaagaaaaatggcctgttcggtaatctgattgcactgtccctgggcctga |
| | cgccgaacttcaaaagcaattttgatctggcagaagatgcgaagctgcaactgagcaaagatacttatgatgacgacctggacaa |
| | tctgttggcacaaatcggtgaccagtatgcagatctgtttctggcggcaaagaacctgtccgatgcgatcctgctgagcgacattct |
| | gcgcgtgaacacggaaattaccaaggctccgctgagcgcgagcatgattaagcgttac |
| Spy-Cas9_2 | ccgctgagcgcgagcatgattaagcgttacgatgagcaccaccaggatctgaccctgctgaaggcgctggtccgtcagcaactg |
| | ccggaaaagtacaaagagattttctttgaccagagcaagaatggctacgcgggctatatcgatggtggcgctagccaagaagag |
| | ttctacaagtttatcaagccgattttggagaaaatggatggtaccgaagagttgctggttaaactgaatcgtgaagatctgctgcgta |
| | agcaacgcacctttgataatggcagcattccgcatcaaattcacctgggtgagttgcatgctatcctgcgccgtcaagaggatttct |
| | acccgtttctgaaagacaaccgtgagaagatcgagaaaattctgactttccgcatcccgtattacgtcggtccgctggcgcgtggt |
| | aacagccgtttcgcatggatgacccgtaagagcgaagaaaccatcaccccatggaacttcgaagaggttgtggataagggtgca |
| | tccgcgcaaagcttcatcgagcgtatgacgaattttgacaagaatctgccgaatgaaaaagtgctgccgaagcacagcctgctgt |
| | acgaatactttaccgtctataacgagctgaccaaagtcaaatacgtcaccgagggtatgcgtaaaccggcgttcctgagcggcga |
| | gcagaagaaggcgattgtcgatctgctgttcaaaacgaatcgtaaagttacggttaagcaactgaaagaggactacttcaagaaa |
| | attgaatgtttcgactctgtcgagattagcggtgttgaagatcgcttcaatgcgagcttgggtacctatcatgatctgctgaagatcat |
| | caaagacaaagatttcctggataatgaagagaacgaggacattctggaagatatcgttttgacgctgaccttgttcgaagatcgtga |
| | gatgatcgaagaacgcctgaaaacgtatgcgcacctgtttgatgataaagtgatgaaacaactgaagcgtcgccgttataccggtt |
| Spy-Cas9_3 | aacaactgaagcgtcgccgttataccggttggggtcgtctgagccgtaagctgatcaacggcattcgtgataaacagtccggtaa |
| | gacgatcctggattttctgaaaagcgacggcttcgcaaaccgtaatttcatgcagctgattcacgacgacagcttgaccttcaaag |
| | aggacatccagaaagcacaagttagcggtcaaggcgatagcctgcatgagcacattgcaaatttggcgggtagcccagcgatc |
| | aagaagggtattctgcagaccgttaaagtggttgatgaactggtgaaagttatgggccgtcacaagcctgaaaacatcgtcattga |
| | gatggcgcgtgaaaatcagaccacgcaaaagggccagaagaatagccgtgaacgcatgaaacgtatcgaagagggcattaaa |
| | gaactgggctcccaaatcctgaaagagcatccggtggagaatactcaactgcagaatgaaaagctgtacctgtactatctgcaaa |
| | acggtcgcgatatgtacgtcgaccaggagctggacatcaaccgcctgtccgactatgacgttgatcacattgtcccgcagagctt |
| | cctgaaagatgacagcatcgacaacaaggtcctgacccgtagcgataagaatcgcggtaaaagcgataacgtgccaagcgaa |
| | gaagtggtgaagaagatgaaaaactattggcgtcaactgttgaacgctaaattgattacgcaacgtaagttcgacaacctgaccaa |
| | ggcggaacgtggtggcctgagcgaactggacaaagcgggtttcatcaagcgccaactggtggaaacccgtcagattacgaaac |
| | atgtcgcccaaattctggacagccgtatgaacacgaagtacgatgaaaacgataaactgattcgtgaagtcaaagttatcacgctg |
| | aaaagcaagctggtgagcgacttccgtaaggattttcagttttacaaagtccgtgaaatcaacaactaccaccatgcgcacgatgc |
| | ctatctgaacgctgt |
| Spy-Cas9_5 | ccatgcgcacgatgcctatctgaacgctgtggtgggtaccgcgctgattaagaagtatccgaaactggaaagcgagttcgtgtac |
| | ggtgattacaaggtttacgatgttcgtaagatgatcgcgaagtccgaacaagaaatcggcaaagcgaccgctaagtatttcttttac |
| | tccaacattatgaactttttcaaaaccgagatcaccctggcaaacggtgagatccgcaaacgtccgctgatcgagactaatggcg |
| | agactggcgaaatcgtgtgggacaaaggtcgtgacttcgccaccgtccgtaaggtattgagcatgccgcaagtcaatattgttaa |
| | gaaaaccgaagttcaaaccggtggtttcagcaaagagagcattctgcctaagcgcaactccgacaaactgattgcccgtaagaa |
| | ggattgggacccgaaaaagtatggcggtttcgatagcccaactgtggcatacagcgtgctggtggttgccaaagtggagaaagg |
| | taagtccaagaagctgaaatctgtcaaagagctgctgggcatcaccattatggagcgcagcagctttgagaaaaatccaatcgac |
| | ttcctggaagcgaagggctacaaagaggtcaagaaagacctgatcatcaagttgccaaagtacagcctgttcgagctggagaat |
| | ggtcgtaagcgcatgctggcctctgccggtgaactgcaaaagggtaacgaactggcgctgccgtcgaaatacgttaactttctgt |
| | acctggcatcccactacgagaaactgaaaggcagccctgaagataacgagcaaaaacaactgtttgttgagcagcacaaacact |
| | atctggatgagatcattgaacagattagcgaattcagcaagcgtgtgatcctggcggacgcgaacctggacaaagtcctgtccgc |
| | gtacaataaacatcgcgacaaaccgattcgtgagcaggcggaaaacattatccacctgtttaccctgacgaatctgggtgcccct |
| | gcggcgtttaagtactttgacactactatcgatcgtaaacgttatacgagcaccaaagaggttctggatgcgaccctgattcaccag |
| | agcattaccggcctgtatgaaacgcgtatcgacctgagccaattgggtggtgaccgctctcgtgcagatccgaaaaagaaacgc |
| | aaagtcgatccgaagaagaagcgcaaggtggacccgaagaaaaagcgtaaagtcggctctaccggtagccgtggctctggttc |
| | gTAActcgagcaccaccaccaccaccactga |

### Construction of Vectors

The bacterial expression vector, pET-21b, was obtained from EMD Millipore (catalog # 69741-3). The pET-21b vector was digested with SwaI/XhoI, and assembled with a linear 143 bp synthetic DNA fragment, 92_6HNLS or 93_6HNLS, using a seamless DNA assembly method following the manufacturer's protocol [Thermo Fisher Scientific, GeneArt™ Seamless Cloning and Assembly Enzyme Mix, catalog # A14606] to produce vector pC9-813-92 and vector pC9-813-93, respectively. The pC9-813-92 and pC9-813-93 vectors were digested with BamHI, and assembled with a PCR-amplified 1501 bp DNA fragment 92P [primer set: AGCGTTGAAGTTCAGCAGCTGAGATCTGTGAAACAAAGCACTATTG (CH1424) and GGACTTTGCGTTTCTTTTTCGGATCCGCAGATGAACCGTGATGGTGATGGTGATG GCTAGAGCCGGAAGCTTTCAGCCCCAGAGCGGCTTTC (CH1425ART-R)] or 93P [primer set: CAGATTAAAATCCGTCTGTTTAGATCTGTGAAACAAAGCACTATTG (CH1425) and GGACTTTGCGTTTCTTTTTCGGATCCGCAGATGAACCGTGATGGTGATGGTGATG GCTAGAGCCGGAAGCTTTCAGCCCCAGAGCGGCTTTC (CH1425ART-R)] from the E. coli MG1655 genome, corresponding to the E. coli alkaline phosphatase (AP) fusion, to generate pC9-813-92P and pC9-813-93P, respectively. The pC9-813-92P vector was digested with BgIII, assembled with a 204 bp synthetic DNA fragment Sp-C9_813-821_CAA, corresponding to the CCAAP box tetramer recombinant antibody (rAb) against Cas9, to generate vector pC9-813-CAA4. The pC9-813-CAA4 vector was digested with BgIII, and self-ligated to remove 117 bp DNA fragment encoding two CCAAP boxes, producing pC9-813-CAA2 which corresponds to the CCAAP box dimer antibody used to detect Cas9. To introduce two mutations, D153G and D330N, into the E. coli AP protein, we PCR-amplified three DNA fragments, P957-1 [primer set: GAATACCTGTTTATTGAAAAATTAAGATCCGGTGGTGGAGGATCAGGATCCGGT GGTGGAGGATCAGGATCTGTGAAACAAAGCACTATTG (CH1483ART-F) and CAGCGCAGCGGGCGTGGCACCCTGCAACTCTGCGGTAG (CH1486)], P957-2 [primer set: CTACCGCAGAGTTGCAGGGTGCCACGCCCGCTGCGCTG (CH1487) and CAAGGATTCGCAGCATGATTCTGTTTATCGATTGACGCAC (CH1492)], and P957-3 [primer set: GTGCGTCAATCGATAAACAGAATCATGCTGCGAATCCTTG (CH1493) and GTGCTCGAGTTTCAGCCCCAGAGCGGCTTTCATG (CH1494)] and assembled to produce a 1,473-bp DNA fragment corresponding to the mutant AP (or P957). This PCR product was digested with BamHI and XhoI, and ligated into BgIII/XhoI digested pC9-813-CAA2, to generate p813C2-P957dB. For the production of the recombinant antibodies (rAbs), two synthetic DNA fragments, Anti-Bace1 (130 bp) and Anti-PDGFR (130 bp) (Table 7), were digested with SwaI/BgIII and ligated into the same enzyme site of the pC9-813-CAA2, to generate pAnti-Bace1-P and pAnti-PDGFR-P, respectively. Four synthetic DNA fragments, Anti-Brca1 (124 bp), Anti-Hsp90 (124 bp), Anti-EstR (124 bp), and Anti-Xiap (124 bp) (Table 7), were digested with SwaI/BgIII and ligated into the SwaI/BamHI sites of the p813C2-P957dB, to generate pAnti-Brca1-P957, pAnti-Hsp90-P957, pAnti-EstR-P957, and pAnti-Xiap-P957, respectively. To produce the recombinant Cas9 protein, pET-Spy-Cas9_d6H vectors were constructed by assembling five parts with overlapping DNA ends using the seamless DNA assembly kit. Briefly, four insert parts [a 1000 bp Spy-Cas9_1, a 1030 bp Spy-Cas9_2, a 1030 bp Spy-Cas9_3, and a 1303 bp Spy-Cas9_5, corresponding to the tagless Cas9] (Table 7) and the SwaI/XhoI-digested pET-21b were assembled, to create pET-Spy-Cas9_d6H.

### Protein Production and Purification

For recombinant protein production, BL21 Star (DE3) cells harboring an expression vector were grown to mid-log phase (optical density at 600 nm [OD600] of 0.6) in LB medium [ampicillin (Amp), 100 μg/ml] at 28° C. and induced with 1 mM IPTG (isopropyl-β-D-thiogalactopyranoside) for 5 h. Cells were harvested by centrifugation at 3000×g for 10 min. Harvested cells were disrupted using a chemical lysis method following the manufacturer's protocol [Thermo Fisher Scientific, BPER™ Complete Bacterial Protein Extraction Reagent, catalog # 89821]. Cell debris and insoluble proteins in the lysate were separated by centrifugation at 16,000×g for 5 minutes. His-tagged recombinant proteins were purified via metal-affinity chromatography using Dynabeads™ His-Tag Isolation and Pulldown beads following the manufacturer's protocol [Thermo Fisher Scientific, catalog # 10103D]. Recombinant Cas9 proteins were purified using the HiTrap Heparin HP column [GE Healthcare, catalog # 17-0406-01] as previously described (Karvelis et al. 2015).

### Dot Blot and Western Blot Analyses

For dot blot analysis, 2 μl (5 μg) of samples were spotted onto a nitrocellulose (NC) membrane and dried completely. Then, non-specific sites were blocked by soaking the membrane in blocking solution [Thermo Fisher Scientific, WesternBreeze™ Blocker/Diluent (Part A and B), catalog # WB7050] for 1 hr at room temperature (or up to 72 hr at 4° C.). The membrane was washed twice with water (1 ml per cm2 of membrane), and incubated with the 1^{st} antibody (Ab) in a binding/wash (BW) buffer [50 mM sodium phosphate, pH 8.0, 300 mM NaCl, and 0.01% Tween 20] for 1 hr at room temperature. The membrane was washed 4 times (2 minutes per wash) with wash buffer [Thermo Fisher Scientific, WesternBreeze™ Wash Solution, catalog # WB7003]. If the 1^{st} Ab was Anti-Cas9 Ab-HRP conjugate [Thermo Fisher Scientific, catalog # MAC133P] or the peptide-AP fusions (2^{nd} Ab not required), the membrane was washed twice with water, and incubated with a chromogenic substrate: Chromogenic Substrate (TMB) [Thermo Fisher Scientific, catalog # WP20004] for HRP and NBT/BCIP substrate solution for AP [Thermo Fisher Scientific, catalog # 34042]. Otherwise, the membrane was incubated with 2^{nd} Ab in the blocking solution for 1 hr. To detect His-tagged peptide and proteins, the Anti-6His Ab-HRP conjugate [Thermo Fisher Scientific, catalog # 46-0707] was used as 2^{nd} Ab. Then the membrane was washed four times with the wash buffer and two times with water. Finally, the blot was incubated with the chromogenic substrates. For the western blot analysis, the protein samples were resolved in 4-20% gradient SDS-PAGE gel, transferred to an NC membrane, and analyzed using the same method for the dot blot analysis [note: we have obtained the best result with a long blocking time (72 hr at 4° C.)].

### Digital Image Processing and Analysis

For the image processing, we used Adobe Photoshop 7.0. Quantitative image analysis of the digital images was carried out using measuring tools of imaging software ImageJ (Schneider et al. 2012). Image analysis results were calculated by averaging data from three independent experiments.

### Statistical Analysis

Statistical analyses were performed using a one-way analysis of variance (ANOVA) and confirmed by Student's t-test [two tails, two-sample equal variance (homoscedastic)]. p values<0.05 considered statistically significant, and scored with five different levels: ♦, p<0.05; ♦♦, p<0.01; ♦♦♦, p<0.001; ♦♦♦♦, p<0.0001; and; ♦♦♦♦♦, p<0.00001. All graphs display mean±SD.

### Results and Discussion

### Physicochemical and Stereochemical Features of the Complementary Amino Acid Pairing (CAAP)

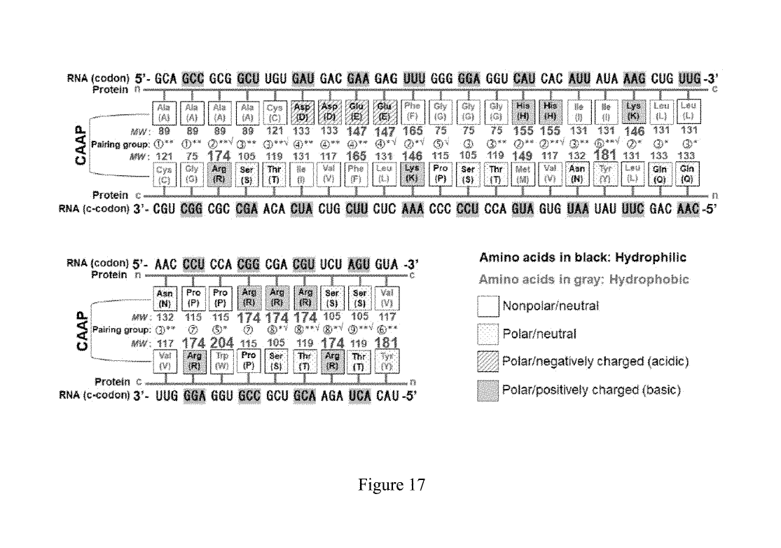

In the present study, we demonstrate that the pairing between two amino acids encoded by a codon and the reverse complementary codon (c-codon) is favored in PPI. We name this pairing the “Complementary Amino Acid Pairing (CAAP).” We summarize all possible CAAPs in FIG. 17. Based on the side chain hydrophobicity and polarity, we categorize CAAP interactions (↔) into the following groups: \textcircled{1}, hydrophobic (nonpolar/neutral) ↔ hydrophobic (nonpolar/neutral) [6.9%]; \textcircled{2}, hydrophobic (nonpolar/neutral) ↔ hydrophilic (polar/positively charged) [17.2%]; \textcircled{3}, hydrophobic (nonpolar/neutral) ↔ hydrophilic (polar/neutral) [27.6%]; \textcircled{4}, hydrophobic (nonpolar/neutral) ↔ hydrophilic (polar/negatively charged) [13.8%]; \textcircled{5}, hydrophobic (nonpolar/neutral) ↔ hydrophilic (nonpolar/neutral) [6.9%]; \textcircled{6}, hydrophobic (nonpolar/neutral) ↔ hydrophobic (polar/neutral) [6.9%]; \textcircled{7}, hydrophilic (nonpolar/neutral) ↔ hydrophilic (polar/positively charged) [6.9%]; \textcircled{8}, hydrophilic (nonpolar/neutral) ↔ hydrophilic (polar/positively charged) [7.9%]; \textcircled{9}, hydrophilic (nonpolar/neutral) ↔ hydrophilic (polar/neutral) [3.4%]. According to our categorization, group \textcircled{1} and \textcircled{6} pairings (A-C, A-G, I-Y, and V-Y) possess hydrophobic interactions, while group \textcircled{8} and \textcircled{9} pairings (2 R-S, R-T, and S-T) may form hydrogen bonds. Some of the group \textcircled{2} and \textcircled{3} pairings involve charge transfer complexing (F-K) and hydrogen bonding (A-R and C-T). However, most of the group \textcircled{2} and \textcircled{3} (2 L-Q, A-S, D-I, D-V, E-F, G-S, G-T, H-M, I-N, L-K, and N-V) and group \textcircled{7} (2 P-R) pairings have not been systematically evaluated for intermolecular interactions before. Interestingly, 38% of CAAP interactions in FIG. 17 (√ group) belong to the group of 26 probable amino acid pairings that can be formed. In addition, we found that 65% of the CAAP interactions are favored amino acid pairs [Relative Frequency (RF)>1.0] in parallel β-strand interactions and 88% favored in antiparallel strands. Moreover, CAAP interactions have been shown to possess favorable stereochemistry. In the stereochemical analysis, amino acids are grouped into three molecular-weight (MW) tiers: small [MW range: 75-133 kDa], medium [MW range: 146-165 kDa], and large [MW range: 174-204 kDa]. Based on this grouping, the CAAP interactions appeared to have small-small (48.3%), small-medium (10.3%), small-large (27.6%), medium-medium (13.8%), and large-large (0%) (FIG. 17). Notably, all high molecular weight (large) residues with bulky side chains such as Arg (R), Tyr (Y), and Trp (W) tend to pair with low molecular weight (small) residues with small side chains, while there is no CAAP interaction between high molecular weight residues (FIG. 17). Therefore, the CAAP interactions may have a spatial flexibility at the PPI interface. These observations lead us to postulate that the physicochemical and stereochemical natures of the CAAP relationships between two polypeptide chains may provide an attractive environment for PPI.

### The CAAP Interactions are Clustered in All PPI Sites

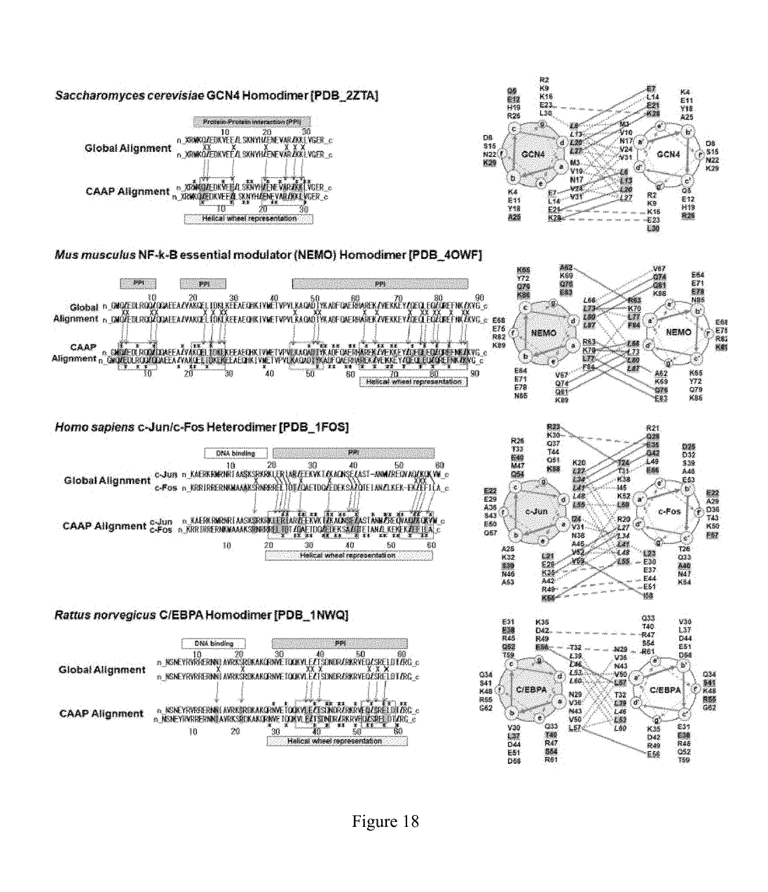

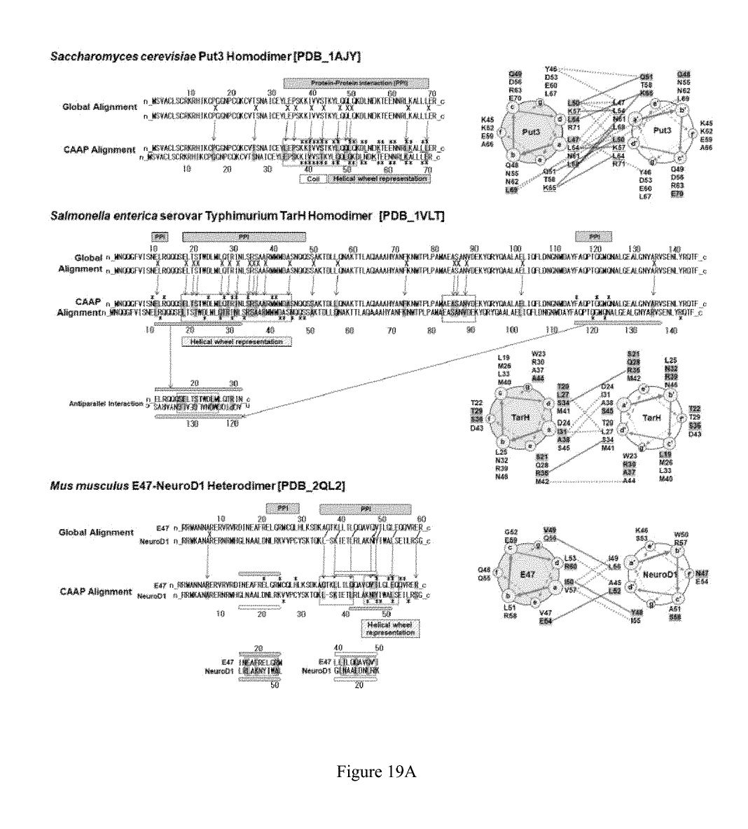

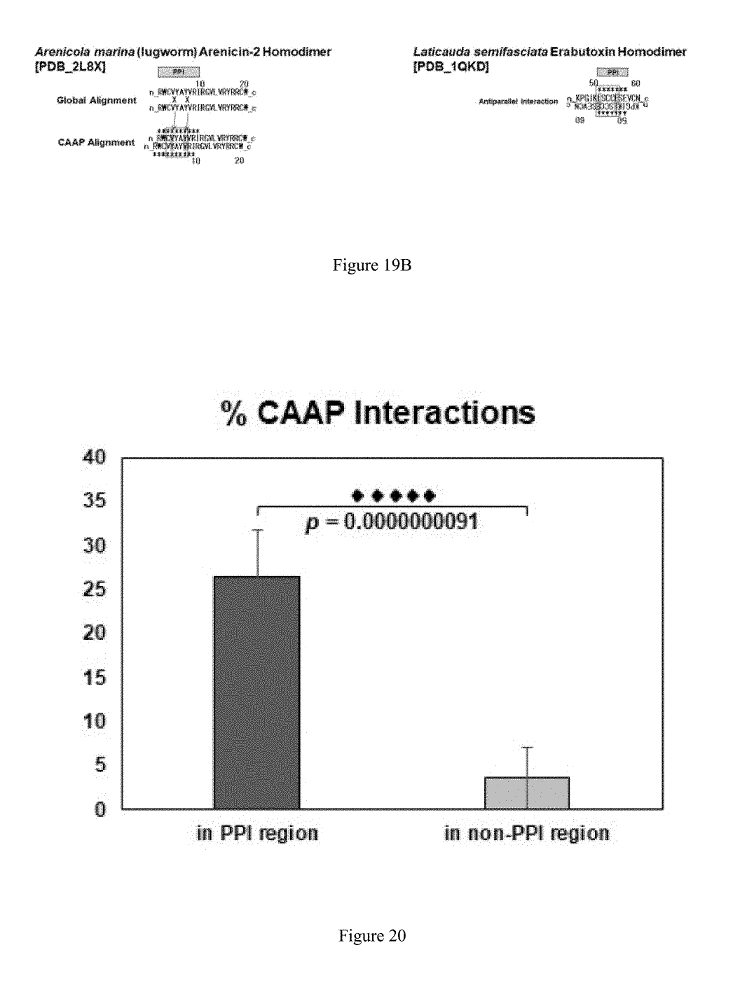

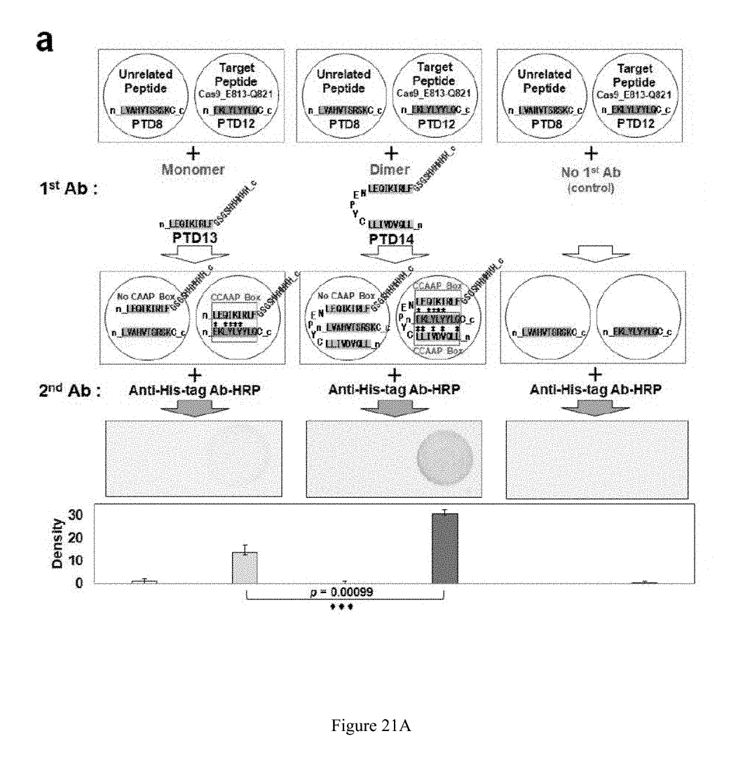

To address the CAAP hypothesis for PPI, we first focused on finding the CAAP interactions in the PPI structure database from the Protein Data Bank (PDB). We examined the well-known leucine zipper proteins: Saccharomyces cerevisiae GCN4/GCN4 homodimer [PDB_2ZTA], Mus musculus NF-k-B essential modulator (NEMO) homodimer [PDB_4OWF], and Homo sapiens c-Jun/c-Fos heterodimer [PDB_1FOS], and Rattus norvegicus C/EBPA homodimer [PDB_1NWQ] (FIG. 18). We also examined five non-leucine-zipper proteins which include three helix-helix (FIG. 19A) and two β-sheet-β-sheet (FIG. 19B) interactions: Saccharomyces cerevisiae Put3 homodimer [PDB_1AJY], Salmonella enterica serovar Typhimurium TarH homodimer [PDB_1VLT], Mus musculus E47-NeuroD1 heterodimer [PDB_2QL2], Arenicola marina (lugworm) Arenicin-2 homodimer [PDB_2L8X], and Laticauda semifasciata Erabutoxin homodimer [PDB_1QKD]. We first determined the linear sequence representation of the dimers' protein sequences (FIGS. 18 and 19A-B). In the global alignment for the parallel interactions, the dimer molecules are aligned to obtain optimal homology matching. For the antiparallel interaction, however, global alignment is not applicable (FIG. 19B). During CAAP alignment, dimer molecules are aligned such that CAAP interactions largely agree with PDB PPI structure data, which we confirmed was when the dimers were shifted by one amino acid from each other in the global alignments (FIGS. 18 and 19A-B). In the global alignments, we did not see any clusters of CAAP interactions in (FIGS. 18 and 19A-B). Interestingly, however, we found that CAAP interactions at n^{chainA}/n+1^{chainB} and/or n+1^{chainA}/n^{chain B} positions in the global alignment (FIGS. 18 and 19A-B). These CAAP interactions are marked with X, /, or \ between the dimer molecules in the global alignments of the linear representations (FIGS. 18 and 19A-B). In the CAAP alignment, CAAP interactions (gray highlight) were revealed when dimers were shifted by one amino acid from each other in the global alignments (FIGS. 18 and 19A-B). Clusters of CAAP residues are enclosed by a gray box called “CCAAP box”. CCAAP boxes enclose eight or more amino acid pairings for the helix-helix, helix-coil, and coil-coil interactions and five or more amino acid pairings for the β-sheet-β-sheet and β-sheet-coil interactions where at least 37.5% are CAAPs. We set this CCAAP box criteria after discovering that a CCAAP box with 37.5% or higher CAAP content does not randomly occur in the non-PPI areas (FIGS. 18 and 19A-B). In the CAAP alignments of the nine dimer proteins (FIGS. 18 and 19A-B), we found 21 CCAAP boxes. Interestingly, 20 out of 21 CCAAP boxes are found in the PPI sites (FIGS. 18 and 19A-B). In addition, all PPI sites are corresponded to at least one CCAAP box (FIGS. 18 and 19A-B). Conversely, we found only one CCAAP box in the non-PPI area of the TarH Homodimer [PDB_1VLT] (FIG. 19A-B). Importantly, the clustered appearance of the CAAP interactions in the PPI sites is statistically significant (FIG. 20, Table 9). We then translated the linear sequence representation to its helical wheel representation to simulate the hypothesized α-helix structural configuration of the residues (FIGS. 18 and 19A). The dimerization angle (topology) of the two interacting molecules in the helical wheel representation was adjusted to build a realistic simulation by comparing it with the PDB structure data. All helical wheel representations provided the best representation with the canonical coiled-coil dimer topology. In the helical wheel representation, we found that 50% of CAAP interactions in the linear representation are clearly aligned at the interface of the two interacting helices (FIGS. 18 and 19B). The helical wheel representation also revealed new CAAP interactions (underline) that could not be identified in the linear representations (FIGS. 18 and 19B). Conversely, 50% (dotted underline) of the CAAP residues in the linear representation were too far apart from each other to possibly form intermolecular interactions in the helical wheel representations (FIGS. 18 and 19B). The PDB PPI structure data revealed that clustered CAAP interactions (CCAAP boxes) in the linear representation are at least partly involved in PPI (FIGS. 18 and 19A-B). A common feature of the helical representation is the presence of hydrophobic interactions at core interfaces. Notably, we also found that many amino acids in the PPI interface likely interact with more than one amino acid in <4 Å distance (FIGS. 18 and 19A-B).

We also investigated 75 additional PPI structures for CCAAP interactions (Table 8). A total of 84 protein structures were selected for their relatively simple PPI structures, which limit the effect of any other potential parameters. Protein structures were also categorized according to parallel or antiparallel alignment. We found CCAAP boxes in all PPI sites in the 82 structure data from PDB (Table 8). However, we could not find any CCAAP box from PPI sites of two dimers: Homo sapiens ERBB2-EGFR heterodimer [PDB_2KS1] and Bos taurus If1 homodimer [PDB_1GMJ]. Interestingly, the PPI sites of these two dimers have a high content of either charged amino acids [PDB_2KS1] or hydrophobic amino acids [PDB_1GMJ]. We found 79 CCAAP boxes in the parallel (↓↓) interactions (76 helix/helix, 2 β-sheet/coil, and 1 β-sheet/β-sheet interactions) and 81 CCAAP boxes in antiparallel (↓↓) interactions (67 helix/helix and 14 β-sheet/β-sheet interactions) (Table 8). Notably, 93% of the β-sheet/β-sheet interactions are antiparallel interactions.

TABLE 8

| Protein | | | | Pairing | |
|---|---|---|---|---|---|
| (chain_structure) | Interaction | PDB ID | CCAAP Box^{a} | Orientation | Source |
| CD2 (chain A_beta | Homo dimer | 1A6P | TYNVT | Antiparallel | Rattus norvegicus |
| sheet 5) | | | GREWR | | |
| CD2 (chain B_beta | | | | | |
| sheet 1) | | | | | |
| HDAg (chain | Homo | 1A92 | LEELERDLRKLK | Antiparallel | Hepatitis delta |
| A_helix 1) | octamer | | KLKRLDRELEEL | | virus |
| HDAg (chain | | | | | |
| B_helix 1) | | | | | |
| Put3 (chain | Homo dimer | 1AJY | LEPSKKIVVSTKYLQQLQ | Parallel | Saccharomyces |
| A_helix) Put3 | | | EPSKKIVVSTKYLQQLQK | | cerevisiae |
| (chain B_helix) | | | | | |
| Cytochrome C | Homo dimer | 1BBH | LSPEEQIE | Antiparallel | Allochromatium |
| (chain A_helix 1) | | | KGMNWGMF | | vinosum |
| Cytochrome C | | | | | |
| (chain B_helix 1) | | | | | |
| TAF(II)-18 (chain | Hetero dimer | 1BH8 | LFSKELRC | Antiparallel | Homo sapiens |
| A_helix 1) | | | EYRNLQEE | | |
| TAF(II)-28 (chain | | | | | |
| B_helix 1) | | | | | |
| TAF(II)-18 (chain | Hetero dimer | 1BH8 | LEDLVIEFITEMTH | Antiparallel | Homo sapiens |
| A_helix 2) | | | EVVEGVFVKSIGSM | | |
| TAF(II)-28 (chain | | | | | |
| B_helix 3) | | | | | |
| ATF4 (chain | Hetero dimer | 1CI6 | LTGECKELEK ETQHKVLELT | Parallel | Mus musculus |
| A_helix 1) | | | | | |
| C/EBP beta (chain | | | | | |
| B_helix 1) | | | | | |
| ATF4 (chain | Hetero dimer | 1CI6 | LKERADSL | Parallel | Mus musculus |
| A_helix 1) | | | RLQKKVEQ | | |
| C/EBP beta (chain | | | | | |
| B_helix 1) | | | | | |
| ATF4 (chain | Hetero dimer | 1CI6 | QYLKDLIE | Parallel | Mus musculus |
| A_helix 1) | | | LSTLRNLF | | |
| C/EBP beta (chain | | | | | |
| B_helix 1) | | | | | |
| c-Jun (chain | Hetero dimer | 1FOS | KLERIARLE | Parallel | Homo sapiens |
| F_helix 2) | | | RELTDTLQA | | |
| c-Fos (chain | | | | | |
| E_helix 2) | | | | | |
| c-Jun (chain | Hetero dimer | 1FOS | LKAQNSEL | Parallel | Homo sapiens |
| F_helix 2) c-Fos | | | EDEKSALQ | | |
| (chain E_helix 2) | | | | | |
| c-Jun (chain | Hetero dimer | 1FOS | VAQLKQKV | Parallel | Homo sapiens |
| F_helix 2) | | | EKLEFILA | | |
| c-Fos (chain | | | | | |
| E_helix 2) | | | | | |
| Domain-Swapped | Homo dimer | 1G6U | PEELAALESE GKLAQLKSKL | Antiparallel | Domain- |
| (chain A_he1ix2) | | | | | Swapped |
| Domain-Swapped | | | | | |
| (chain B_he1ix2) | | | | | |
| Domain-Swapped | Homo dimer | 1G6U | LEKKLAAL | Antiparallel | Domain- |
| (chain A_he1ix2) | | | KKELAQLE | | Swapped |
| Domain-Swapped | | | | | |
| (chain B_he1ix2) | | | | | |
| Gal4 (chain | Homo dimer | 1HBW | RLERLEQL | Parallel | Saccharomyces |
| A_helix 1) | | | SRLERLEQ | | cerevisiae |
| Gal4 (chain | | | | | |
| B_helix 1) | | | | | |
| Human Lectin | Homo dimer | 1HLC | SSFKL | Antiparallel | Homo sapiens |
| (chain A_beta sheet | | | KLKFS | | |
| 13) | | | | | |
| Human Lectin | | | | | |
| (chain B_beta sheet | | | | | |
| 13) | | | | | |
| Ala-14 (chain | Homo trimer | 1JCD | ARANQRAD | Parallel | Escherichia coli |
| A_helix) | | | AARANQRA | | |
| Ala-14 (chain | | | | | |
| B_helix) | | | | | |
| c-Jun (chain | Homo dimer | 1JUN | KAQNSELAST | Parallel | Homo sapiens |
| A_helix) | | | LKAQNSELAS | | |
| c-Jun (chain | | | | | |
| B_helix) | | | | | |
| Nsp3 (chain | Homo dimer | 1LJ2 | MHSLQNVI | Parallel | Simian rotavirus |
| A_helix 1) | | | HSLQNVIP | | A/SA11 |
| Nsp3 (chain | | | | | |
| B_helix 1) | | | | | |
| Nsp3 (chain | Homo dimer | 1LJ2 | ELQVYNNKLERDLQNKIGSLT | Parallel | Simian rotavirus |
| A_helix 1) | | | LQVYNNKLERDLQNKIGSLTS | | A/SA12 |
| Nsp3 (chain | | | | | |
| B_helix 1) | | | | | |
| Tpm1 (chain | Homo dimer | 1MV4 | IDDLEDELYAQKL | Parallel | Rattus norvegicus |
| A_helix1) | | | DDLEDELYAQKLK | | |
| Tpm1 (chain | | | | | |
| B_helix1) | | | | | |
| Arc (chain A_coil) | Homo dimer | 1MYL | MPQFNLRW | Antiparallel | Bacteriophage |
| Arc (chain B_coil) | | | WRLNFQPM | | P22 |
| Myc (chain A_helix | Hetero dimer | 1NKP | LRKRREQL | Parallel | Homo sapiens |
| 1) | | | KRQNALLE | | |
| Max (chain B_helix | | | | | |
| 1) | | | | | |
| C/EBPA (chain | Homo dimer | 1NWQ | KVLELTSD | Parallel | Rattus norvegicus |
| A_helix 1) | | | VLELTSDN | | |
| C/EBPA (chain | | | | | |
| B_helix 1) | | | | | |
| C/EBPA (chain | Homo dimer | 1NWQ | EQLSRELD | Parallel | Rattus norvegicus |
| A_helix 2) | | | QLSRELDT | | |
| C/EBPA(chain | | | | | |
| B_helix 2) | | | | | |
| Erabutoxin (chain | Homo dimer | 1QKD | LSCCE | Antiparallel | Laticauda |
| A_beta sheet 5) | | | ECCSL | | semifasciata |
| Erabutoxin (chain | | | | | |
| B_beta sheet 5) | | | | | |
| Max (chain A_helix | Homo dimer | 1R05 | SFHSLRDS | Parallel | Homo sapiens |
| 1 | | | DKATEYIQ | | |
| Max (chain B_helix | | | | | |
| 2) | | | | | |
| Max (chain A_helix | Homo dimer | 1R05 | VHTLQQDIDDLK | Parallel | Homo sapiens |
| 2) | | | HTLQQDIDDLKR | | |
| Max (chain B_helix | | | | | |
| 2) | | | | | |
| Max (chain A_helix | Homo dimer | 1R05 | LEQQVRAL | Parallel | Homo sapiens |
| 2) | | | EQQVRALE | | |
| Max (chain B_helix | | | | | |
| 2) | | | | | |
| Geminin (chain | Homo dimer | 1T6F | DNEIARLK | Parallel | Homo sapiens |
| A_helix 1) | | | NEIARLKK | | |
| Geminin (chain | | | | | |
| B_helix 1) | | | | | |
| Endothelin-1 (chain | Homo dimer | 1T7H | RCSCS | Antiparallel | Homo sapiens |
| A_beta sheet) | | | SCSCR | | |
| Endothelin-1 (chain | | | | | |
| B_beta sheet) | | | | | |
| Cenp-b (chain | Homo dimer | 1UFI | GEAMAYFA | Antiparallel | Homo sapiens |
| A_helix 1) | | | AFYAMAEG | | |
| Cenp-b (chain | | | | | |
| B_helix 1) | | | | | |
| Cenp-b (chain | Homo dimer | 1UFI | FPIDDRVQ | Antiparallel | Homo sapiens |
| A_helix 2) | | | KRTVHVLD | | |
| Cenp-b (chain | | | | | |
| B_helix 2) | | | | | |
| PALS-1-L27N | Hetero dimer | 1VF6 | LQVLDRLK | Antiparallel | Homo sapiens |
| (chain A_helix 1) | | | SIDEQSQS | | Mus musculus |
| PATJ-L27 (chain | | | | | |
| B_helix 2) | | | | | |
| TarH (chain | Homo dimer | 1VLT | ELTSTWDLMLQTRINLSRSAARM | Parallel | Salmonella |
| A_helix 1) | | | MMDA | | enterica serovar |
| TarH (chain | | | LTSTWDLMLQTRINLSRSAARMM | | Typhimurium |
| B_helix 1) | | | MDAS | | |
| TarH (chain | Homo dimer | 1VLT | SELTSTWDLM GLAEGLANQM | Antiparallel | Salmonella |
| A_helix 1) | | | | | enterica serovar |
| TarH (chain | | | | | Typhimurium |
| B_helix4) | | | | | |
| Gemin6 (chain | Hetero dimer | 1Y95 | LTTDPVSA | Parallel | Homo sapiens |
| A_beta sheet 3) | | | ALRERYLR | | |
| Gemin7 (chain | | | | | |
| B_Helix 1) | | | | | |
| Gemin6 (chain | Hetero dimer | 1Y95 | SMSVTGI | Antiparallel | Homo sapiens |
| A_beta sheet 5) | | | KFTYSII | | |
| Gemin7 (chain | | | | | |
| B_beta sheet 7) | | | | | |
| Med7 (chain | Hetero dimer | 1YKH | LKSLLLNY | Antiparallel | Saccharomyces |
| A_helix 1) | | | IQRTKLII | | cerevisiae |
| Srb7 (chain B_helix | | | | | |
| 2) | | | | | |
| Med7 (chain | Hetero dimer | 1YKH | IHHLLNEY | Parallel | Saccharomyces |
| A_helix 2) | | | ETMQDLCI | | cerevisiae |
| Srb7 (chain B_helix | | | | | |
| 1) | | | | | |
| Med7 (chain | Hetero dimer | 1YKH | LEEQLEYK | Parallel | Saccharomyces |
| A_helix 3) | | | MLQKKLVE | | cerevisiae |
| Srb7 (chain B_helix | | | | | |
| 3) | | | | | |
| Lin-7 (chain | Hetero dimer | 1ZL8 | QRILELMEHVQ LIRKLEKADNN | Antiparallel | Caenorhabditis |
| A_helix 1) | | | | | elegans Homo |
| Lin-2 (chain | | | | | sapiens |
| B_helix 2) | | | | | |
| Lin-7 (chain | Hetero dimer | 1ZL8 | NNAKLASL | Antiparallel | Caenorhabditis |
| A_helix 2) | | | ELVEKARQ | | elegans Homo |
| Lin-2 (chain | | | | | sapiens |
| B_helix 1) | | | | | |
| DSX (chain | Homo dimer | 1ZV1 | MPLMYVIL | Antiparallel | Drosophila |
| A_helix 3) | | | SAEEINAD | | melanogaster |
| DSX (chain | | | | | |
| B_helix 2) | | | | | |
| cGMP-dependent | Homo dimer | 1ZXA | EIQELKRK | Parallel | Homo sapiens |
| protein kinase | | | IQELKRKL | | |
| (chain A_helix) | | | | | |
| Usp8 (chain | Homo dimer | 2A9U | SVPKELYL | Parallel | Homo sapiens |
| A_coil) Usp8 | | | LDRDEERA | | |
| (chain B_helix 2) | | | | | |
| Usp8 (chain | Homo dimer | 2A9U | RDEERAYVLY ELYLSSSLKD | Parallel | Homo sapiens |
| A_helix2) | | | | | |
| Usp8 (chain | | | | | |
| B_coil) | | | | | |
| DP1 (chain A_helix | Hetero dimer | 2AZE | QNLEVERQ | Parallel | Homo sapiens |
| 1) | | | LEGLTQDL | | |
| E2F1 (chain | | | | | |
| B_helix 1) | | | | | |
| DP1 (chain A_helix | Hetero dimer | 2AZE | IAFKNLVQ | Parallel | Homo sapiens |
| 1) | | | LRLLSEDT | | |
| E2F1 (chain | | | | | |
| B_helix 1) | | | | | |
| DP1 (chain A_beta | Hetero dimer | 2AZE | FIIVN | Antiparallel | Homo sapiens |
| sheet 1) | | | KIVMV | | |
| E2F1 (chain B_beta | | | | | |
| sheet 1) | | | | | |
| Beta-myosin S2 | Homo dimer | 2FXO | EFTRLKEALEKSEARRKEL | Parallel | Homo sapiens |
| (chain A_helix 1) | | | FTRLKEALEKSEARRKELE | | |
| Beta-myosin S2 | | | | | |
| (chain B_helix 1) | | | | | |
| Beta-myosin S2 | Homo dimer | 2FXO | LQEKNDLQL | Parallel | Homo sapiens |
| (chain A_helix 2) | | | QEKNDLQLQ | | |
| Beta-myosin S2 | | | | | |
| (chain B_helix 2) | | | | | |
| Beta-myosin S2 | Homo dimer | 2FXO | KLEDECSELKRDIDDLE | Parallel | Homo sapiens |
| (chain A_helix 3) | | | LEDECSELKRDIDDLEL | | |
| Beta-myosin S2 | | | | | |
| (chain B_helix 3) | | | | | |
| Phe-14 (chain | Homo | 2GUV | KDDFARFNQR FNAFRSDFQA | Parallel | Escherichia coli |
| A_helix) | pentamer | | | | |
| Phe-14 (chain | | | | | |
| B_helix) | | | | | |
| ROM (chain | Homo dimer | 2IJK | ADEQADICE | Antiparallel | Escherichia coli |
| A_helix 2) | | | RALCSRYLE | | |
| ROM (chain | | | | | |
| B_helix 2) | | | | | |
| Hi0947 (chain | Homo dimer | 2JUZ | LEKHKAPVDLS ELVAIMDNVIA | Antiparallel | Haemophilus |
| A_helix 1-2) | | | | | influenzae |
| Hi0947 (chain | | | | | |
| B_helix 1) | | | | | |
| Hi0947 (chain | Homo dimer | 2JUZ | SLIALGNMA | Antiparallel | Haemophilus |
| A_helix 2) | | | AMNGLAILS | | influenzae |
| Hi0947 (chain | | | | | |
| B_helix 2) | | | | | |
| Hi0947 (chain | Homo dimer | 2JUZ | EALAQAFSNSL LSNSFAQALAE | Antiparallel | Haemophilus |
| A_helix 3) | | | | | influenzae |
| Hi0947 (chain | | | | | |
| B_helix 3) | | | | | |
| Arenicin-2 (chain | Homo dimer | 2L8X | CVYAY | Parallel | Arenicola marina |
| A_beta sheet 1) | | | VYAYV | | (lugworm) |
| Arenicin-2 (chain | | | | | |
| B_beta sheet 1) | | | | | |
| Erbb4 (chain | Homo dimer | 2LCX | ARTPLIAA | Parallel | Homo sapiens |
| A_helix1) | | | RTPLIAAG | | |
| Erbb4 (chain | | | | | |
| B_helix1) | | | | | |
| FGFR3 (chain | Homo dimer | 2LZL | AGSVYAGI | Parallel | Homo sapiens |
| A_helix 1) | | | EAGSVYAG | | |
| FGFR3 (chain | | | | | |
| B_helix 1) | | | | | |
| Xcl1 (chain A_beta | Homo dimer | 2N54 | CVSLT | Antiparallel | Homo sapiens |
| sheet 1) | | | TLSVC | | |
| Xcl1 (chain B_beta | | | | | |
| sheet 1) | | | | | |
| Xcl1 (chain A_beta | Homo dimer | 2N54 | TYTIT | Antiparallel | Homo sapiens |
| sheet 2) | | | TITYT | | |
| Xcl1 (chain B_beta | | | | | |
| sheet 2) | | | | | |
| CXCL12 (chain | Homo dimer | 2NWG | VKHLKILN | Antiparallel | Homo sapiens |
| A_beta sheet 1) | | | NLIKLHKV | | |
| CXCL12 (chain | | | | | |
| B_beta sheet 1) | | | | | |
| CXCL12 (chain | Homo dimer | 2NWG | IQEYLEKALN NLAKELYEQI | Antiparallel | Homo sapiens |
| A_helix1) | | | | | |
| CXCL12 (chain | | | | | |
| B_helix1) | | | | | |
| Ylan (chain | Homo dimer | 2ODM | EVLDTQMFGLQKEVDFAVK | Parallel | Staphylococcus |
| A_helix 2) | | | LYEEVLDTQMFGLQKEVDF | | aureus subsp. |
| Ylan (chain B_helix | | | | | aureus MW2 |
| 2) | | | | | |
| Ylan (chain | Homo dimer | 2ODM | QLTKDADE | Antiparallel | Staphylococcus |
| A_helix 1) | | | LKVAFDVE | | aureus subsp. |
| Ylan (chain B_helix | | | | | aureus MW2 |
| 2) | | | | | |
| Hy5 (chain | Homo dimer | 2OQQ | GSAYLSEL | Parallel | Arabidopsis |
| A_helix) Hy5 | | | SAYLSELE | | thaliana |
| (chain B_helix) | | | | | |
| Hy5 (chain | Homo dimer | 2OQQ | LENKNSEL | Parallel | Arabidopsis |
| A_helix) Hy5 | | | ENKNSE LE | | thaliana |
| (chain B_helix) | | | | | |
| Hy5 (chain | Homo dimer | 2OQQ | LEERLSTL | Parallel | Arabidopsis |
| A_helix) Hy5 | | | EERLSTLQ | | thaliana |
| (chain B_helix) | | | | | |
| E47 (helix 2) | Hetero dimer | 2QL2 | QVILGLEQ | Parallel | Mus musculus |
| NeuroD1 (helix 2) | | | KNYIWALS | | |
| E47 (chain A_helix | Hetero dimer | 2QL2 | EAFRELGR | Parallel | Mus musculus |
| 1) | | | LAKNYIWA | | |
| NeuroD1 (chain | | | | | |
| B_helix 2) | | | | | |
| E47 (chain A_helix | Hetero dimer | 2QL2 | ILQQAVQV | Parallel | Mus musculu |
| 2) | | | NAALDNLR | | |
| NeuroD1 (chain | | | | | |
| B_helix 1) | | | | | |
| c-Fos (chain | Hetero dimer | 2WT7 | LEDEKSALQ | Parallel | Mus musculus |
| A_helix 1) | | | QLIQQVEQL | | |
| MafB (chain | | | | | |
| B_helix 1) | | | | | |
| Bst2 (chain | Homo dimer | 2XG7 | HKLQDASA | Parallel | Homo sapiens |
| A_helix1) | | | KLQDASAE | | |
| Bst2 (chain | | | | | |
| B_helix1) | | | | | |
| CHMP3 (chain | Hetero dimer | 2XZE | SRLATLRS | Antiparallel | Homo sapiens |
| B_helix 1) | | | SGLQSLAR | | |
| STAMBP (chain | | | | | |
| B_helix 3) | | | | | |
| SCL (chain A_helix | Hetero dimer | 2YPB | AFAELRKL | Parallel | Homo sapiens |
| 2) | | | LILQQAVQ | | |
| E47 (chain B_helix | | | | | |
| 2) | | | | | |
| SCL (chain A_helix | Hetero dimer | 2YPB | NEILRLAMK | Parallel | Homo sapiens |
| 2) | | | DINEAFREL | | |
| E47 (chain B_helix | | | | | |
| 2) | | | | | |
| GCN4 (chain | Homo dimer | 2ZTA | QLEDKVEE | Parallel | Saccharomyces |
| A_helix 2) | | | LEDKVEEL | | cerevisiae |
| GCN4 (chain | | | | | |
| B_helix 2) | | | | | |
| GCN4 (chain | Homo dimer | 2ZTA | LENEVARLKK ENEVARLKKL | Parallel | Saccharomyces |
| A_helix 2) | | | | | cerevisiae |
| GCN4 (chain | | | | | |
| B_helix 2) | | | | | |
| HV1 (chain | Homo dimer | 3A2A | LKQMNVQL | Parallel | Homo sapiens |
| A_helix1) | | | KQMNVQLA | | |
| HV1 (chain | | | | | |
| B_helix1) | | | | | |
| Cce_0567 (chain | Homo dimer | 3CSX | KVRKLNSK | Antiparallel | Cyanobacterium |
| A_helix 1) | | | LTEEWINL | | Cyanothece |
| Cce_0567 (chain | | | | | |
| B_helix 1) | | | | | |
| Cce_0567 (chain | Homo dimer | 3CSX | LHDLAEGL | Antiparallel | Cyanobacterium |
| A_helix 1) | | | ERFIEYTK | | Cyanothece |
| Cce_0567 (chain | | | | | |
| B_helix 1) | | | | | |
| HP0062 (chain | Homo dimer | 3FX7 | EVREFVGHLERF | Antiparallel | Helicobacter |
| A_helix 1) | | | LNHFHNSLSNVE | | pylori |
| HP0062 (chain | | | | | |
| B_helix 1) | | | | | |
| HP0062 (chain | Homo dimer | 3FX7 | RDKFSEVLDNL AIQEQAAEDFE | Antiparallel | Helicobacter |
| A_helix 2) | | | | | pylori |
| HP0062 (chain | | | | | |
| B_helix 2) | | | | | |
| C.esp1396i (chain | Homo dimer | 3G5G | VVFFEMLIKE IEKILMEFFV | Antiparallel | Enterobacter sp. |
| A_helix 5) | | | | | RFL1396 |
| C. esp1396i (chain | | | | | |
| B_helix 5) | | | | | |
| MAPRE1 (chain | Homo dimer | 3GJO | ELMQQVNVLKLTVEDL | Parallel | Homo sapiens |
| A_helix 1) | | | LMQQVNVLKLTVEDLE | | |
| MAPRE1 (chain | | | | | |
| B_helix 1) | | | | | |
| MAPRE1 (chain | Homo dimer | 3GJO | FGKLRNIE | Parallel | Homo sapiens |
| A_helix 1) | | | GKLRNIEL | | |
| MAPRE1 (chain | | | | | |
| B_helix 1) | | | | | |
| Gld1 (chain | Homo dimer | 3K6T | EYLADLVK | Antiparallel | Caenorhabditis |
| A_helix 1) | | | LREVNSFM | | elegans |
| Gld1 (chain | | | | | |
| B_helix 2) | | | | | |
| Rev (chain A_helix | Homo dimer | 3LPH | DEDSLKAVRLIKFLY | Antiparallel | HIV type 1 |
| 1) | | | YLFKILRVAKLSDED | | (HXB3 |
| Rev (chain B_helix | | | | | ISOLATE) |
| 1) | | | | | |
| MinE (chain | Homo dimer | 3MCD | LKLIL | Antiparallel | Helicobacter |
| A_beta sheet 1) | | | ALILK | | Pylori |
| MinE (chain | | | | | |
| B_beta sheet 1) | | | | | |
| Pkg1-Beta (chain | Homo dimer | 3NMD | IDELELELDQKDELIQML | Parallel | Homo sapiens |
| A_helix) | | | DELELELDQKDELIQMLQ | | |
| Pkg1-Beta (chain | | | | | |
| B_helix) | | | | | |
| Swi5 (chain | Homo | 3VIR | QDALAKLKNRDAKQTV | Antiparallel | Schizosaccharomyces |
| A_helix) | tetramer | | LAIDRIENYTHLLDIH | | pombe |
| Swi5(chain | | | | | |
| B_helix) | | | | | |
| Swi5 (chain | Homo | 3VIR | KEQLESSLQDALAKLK | Antiparallel | Schizosaccharomyces |
| A_helix) | tetramer | | KLKALADQLSSELQEK | | pombe |
| Swi5(chain | | | | | |
| C_helix) | | | | | |
| Swi5 (chain | Homo | 3VIR | VQKHIDLLHTYNE | Parallel | Schizosaccharomyces |
| B_helix) | tetramer | | HLLEQQKEQLESS | | pombe |
| Swi5(chain | | | | | |
| C_helix) | | | | | |
| Hv1 (chain A_helix | Homo dimer | 3VMX | LKQINIQL | Parallel | Mus musculus |
| 1) | | | KQINIQLA | | |
| Hv1 (chain B_helix | | | | | |
| 1) | | | | | |
| Sgt2 (chain A_helix | Homo | 3ZDM | EIAALIVNYF | Antiparallel | Saccharomyces |
| 1) | tetramer | | FYNVILAAIE | | cerevisiae |
| Sgt2 (chain B_helix | | | | | |
| 1) | | | | | |
| Sgt2 (chain A_helix | Homo | 3ZDM | ADSLNVAMDCISEAFG | Parallel | Saccharomyces |
| 2) | tetramer | | GFAESICDMAVNLSDA | | cerevisiae |
| Sgt2 (chain B_helix | | | | | |
| 1) | | | | | |
| Cc2-LZ (chain | Homo dimer | 4BWN | QLEDLKQQL | Parallel | Homo sapiens |
| A_helix 1) | | | LEDLKQQLQ | | |
| Cc2-LZ (chain | | | | | |
| B_helix 1) | | | | | |
| Cc2-LZ (chain | Homo dimer | 4BWN | ELLQEQLEQLQREYSKL | Parallel | Homo sapiens |
| A_helix 2) | | | LLQEQLEQLQREYSKLK | | |
| Cc2-LZ (chain | | | | | |
| B_helix 2) | | | | | |
| Qua1 (chain | Homo dimer | 4DNN | TPDYLXQL | Antiparallel | Mus musculus |
| A_helix 2) | | | RSIEEDLL | | |
| Qua1 (chain | | | | | |
| B_helix 2) | | | | | |
| DD_Ribeta_PKA | Homo dimer | 4F9K | KFLREHFEKL LKEFHERLKK | Antiparallel | Homo sapiens |
| (chain A_helix3) | | | | | |
| DD_Ribeta_PKA | | | | | |
| (chain B_helix3) | | | | | |
| Trim25 (chain | Homo dimer | 4LTB | SADLEATLRHKLTVMY | Antiparallel | Homo sapiens |
| A_helix1) | | | DRKTLSQEIEEKLTQI | | |
| Trim25 (chain | | | | | |
| B_helix1) | | | | | |
| Trim25 (chain | Homo dimer | 4LTB | LDDVRNRQ | Antiparallel | Homo sapiens |
| A_helix1) | | | YITDFKSN | | |
| Trim25 (chain | | | | | |
| B_helix1) | | | | | |
| Trim25 (chain | Homo dimer | 4LTB | LRHKLTVMYSQIN | Parallel | Homo sapiens |
| A_helix1) | | | KASKLRGISTKPV | | |
| Trim25 (chain | | | | | |
| B_helix2) | | | | | |
| Trim25 (chain | Homo dimer | 4LTB | VRNRQQDV | Parallel | Homo sapiens |
| A_helix1) | | | HKLIKGIH | | |
| Trim25 (chain | | | | | |
| B_helix2) | | | | | |
| Trim25 (chain | Homo dimer | 4LTB | RKVEQLQQEYTEM | Parallel | Homo sapiens |
| A_helix1) | | | LKNELKQCIGRLQ | | |
| Trim25 (chain | | | | | |
| B_helix2) | | | | | |
| Trim25 (chain | Homo dimer | 4LTB | KNELKQCIGR GICQKLENKL | Antiparallel | Homo sapiens |
| A_helix2) | | | | | |
| Trim25 (chain | | | | | |
| B_helix2) | | | | | |
| Mst1 (chain | Hetero dimer | 4OH8 | LQKRLLAL | Antiparallel | Homo sapiens |
| A_helix) | | | RLAEELKQ | | |
| Rassf5 | | | | | |
| Sarah (chain | | | | | |
| B_helix) | | | | | |
| Naf1 (chain A_beta | Homo dimer | 4OO7 | PLILK | Parallel | Homo sapiens |
| sheet 2) | | | VVNEI | | |
| Naf1 (chain B_coil) | | | | | |
| NEMO(chain | Homo dimer | 4OWF | QLEDLRQQL | Parallel | Mus musculus |
| A_helix 1) | | | LEDLRQQLQ | | |
| NEMO (chain | | | | | |
| B_helix 1) | | | | | |
| NEMO(chain | Homo dimer | 4OWF | KQELIDKL | Parallel | Mus musculus |
| A_helix 1) | | | QELIDKLK | | |
| NEMO (chain | | | | | |
| B_helix 1) | | | | | |
| NEMO(chain | Homo dimer | 4OWF | LKAQADIY | Parallel | Mus musculus |
| A_helix 2) | | | KAQADIYK | | |
| NEMO (chain | | | | | |
| B_helix 2) | | | | | |
| NEMO(chain | Homo dimer | 4OWF | AREKLVEKKEY | Parallel | Mus musculus |
| A_helix 2-3) | | | LQEQLEQLQREFNKL | | |
| NEMO (chain | | | REKLVEKKEYL | | |
| B_helix 2-3) | | | QEQLEQLQREFNKLK | | |
| GBR1 (chain | Hetero dimer | 4PAS | KSRLLEKE | Parallel | Homo sapiens |
| A_helix 1) | | | SRLEGLQS | | |
| GBR2 (chain | | | | | |
| B_helix 1) | | | | | |
| GBR1 (chain | Hetero dimer | 4PAS | EERVSELRHQLQ | Parallel | Homo sapiens |
| A_helix 1) | | | LDKDLEEVTMQL | | |
| GBR2 (chain | | | | | |
| B_helix 1) | | | | | |
| Jip3 (chain A_helix | Homo dimer | 4PXJ | DLIAKVDQ | Antiparallel | Homo sapiens |
| 1) | | | IRNELKVK | | |
| Jip3 (chain B_helix | | | | | |
| 1) | | | | | |
| Pkg1-Alpha (chain | Homo dimer | 4R4M | LKRKLHKLQ | Parallel | Homo sapiens |
| A_helix) | | | ELKRKLHKL | | |
| Pkg1-Alpha (chain | | | | | |
| B_helix) | | | | | |
| VBP (chain | Homo dimer | 4U5T | EIRAAFLE | Parallel | Homo sapiens |
| A_helix) | | | LEIRAAFL | | |
| VBP (chain | | | | | |
| B_helix) | | | | | |
| NBL1 (chain | Homo dimer | 4X1J | GQCFS | Antiparallel | Homo sapiens |
| A_beta sheet 3) | | | SFCQG | | |
| NBL1 (chain | | | | | |
| B_beta sheet 3) | | | | | |
| Gp7-Myh7-EB1 | Homo dimer | 4XA1 | KLEKEKSEFKLELDDVT | Parallel | Homo sapiens |
| (chain A_helix 3) | | | LEKEKSEFKLELDDVTS | | |
| Gp7-Myh7-EB1 | | | | | |
| (chain B_helix 3) | | | | | |
| Gp7-Myh7-EB1 | Homo dimer | 4XA1 | ELGEQIDNL | Parallel | Homo sapiens |
| (chain A_helix 3) | | | LGEQIDNLQ | | |
| Gp7-Myh7-EB1 | | | | | |
| (chain B_helix 3) | | | | | |
| Gp7-Myh7-EB1 | Homo dimer | 4XA1 | LQQLRVNYG QQLRVNYGS | Parallel | Homo sapiens |
| (chain A_helix 2) | | | | | |
| Gp7-Myh7-EB1 | | | | | |
| (chain B_helix 2) | | | | | |
| Gp7-Myh7-EB1 | Homo dimer | 4XA1 | TEALQQLR | Antiparallel | Homo sapiens |
| (chain A_helix 2) | | | LIDEHEEP | | |
| Gp7-Myh7-EB1 | | | | | |
| (chain B_helix 1) | | | | | |
| Sialostatin L (chain | Homo dimer | 4ZM8 | VETQVVAGTNYRLT | Antiparallel | Ixodes scapularis |
| A_coil + beta sheet | | | TLRYNTGAVVQTEV | | |
| 1 & 2) | | | | | |
| Norrin (chain | Homo dimer | 5BQB | ASRSE | Antiparallel | Homo sapiens |
| A_beta sheet 3) | | | GECRA | | |
| Norrin (chain | | | | | |
| B_beta sheet 2) | | | | | |
| Kinesin-like Protein | Homo dimer | 5DJN | LKEKLEESEKLIKEL | Parallel | Mus musculus |
| (chain A_helix1) | | | ELKEKLEESEKLIKE | | |
| Kinesin-like Protein | | | | | |
| (chain B_helix1) | | | | | |
| Kinesin-like Protein | Homo dimer | 5DJN | LESMGISLETSG QLESMGISLETS | Parallel | Mus musculus |
| (chain A_helix1) | | | | | |
| Kinesin-like Protein | | | | | |
| (chain B_helix1) | | | | | |
| Cc1-fha (chain | Homo dimer | 5DJO | LKEKLEES | Parallel | Mus musculus |
| A_helix 1) | | | ELKEKLEE | | |
| Ccl1fha (chain | | | | | |
| B_helix 1) | | | | | |
| Phenylalanine-4- | Homo dimer | 5FII | ALAKVLRL | Antiparallel | Homo sapiens |
| hydroxylase (chain | | | FLRLVKAL | | |
| A_helix1) | | | | | |
| Phage Coat Protein | Homo dimer | 5FS4 | IRTVI | Antiparallel | Acinetobacter |
| (chain A_beta sheet | | | VTRIS | | phage AP205 |
| 5) | | | | | |
| Myosin X (chain | Homo dimer | 5HMO | SLQKLQQL | Parallel | Bos taurus |
| A_helix 2) | | | VEEILRLE | | |
| Myosin X (chain | | | | | |
| C_helix 3) | | | | | |
| Myosin X (chain | Homo dimer | 5HMO | LEKEIEDLQ | Antiparallel | Bos taurus |
| A_helix 2) | | | QLDEIEKEL | | |
| Myosin X (chain | | | | | |
| C_helix 2) | | | | | |
| BLM Helicase | Homo dimer | 5LUS | EQQLYAVMDDICKLVDA | Antiparallel | Pelecanus crispus |
| (chain A_helix 1) | | | ALLKRRLGRQLLLEKAC | | Bruch, 1832 |
| BLM Helicase | | | | | |
| (chain A_helix 2) | | | | | |
| Ncd (chain | Homo dimer | 5W3D | AELETCKEQL ELETCKEQLF | Parallel | Drosophila |
| A_helix1) | | | | | melanogaster |
| Ncd (chain | | | | | |
| B_helix1) | | | | | |

^{a}CAAP interactions underlined

### Designing Synthetic Antibodies (sAbs) using the CCAAP Principle

We assessed the composition of all amino acid pairings in the CCAAP boxes (Table 8) to obtain information on pairing preference and how the CAAPs were spaced out in the CCAAP box, which may be important factors for binding affinity, specificity, and stability. The raw abundance numbers are shown in Table 9 and summarized in FIG. 4A-B. This data was then used for designing an oligopeptide synthetic antibody (sAb) sequence that can interact with a target polypeptide sequence of a protein. The general rule was to design the sAb sequence such that it forms a CCAAP box in the PPI with the target sequence. For the spacing, we tried to mimic some CCAAP box examples covering diverse spacing patterns (Table 8): OXXOXOXOO [PDB_1YKH], OXOOOOXXX [PDB_3NMD], OXOOOOXO [PDB_4ZM8], OOXOOXOO [PDB_3VIR], OOXOOOXOO [PDB_4BWN], OOXXOOXO [PDB_3VMX], OOOXOXOOO [PDB_2WT7], and OOOOOXOOOO [PDB_4XA1] (O stands for a CAAP interaction residue, X stands for a non-CAAP interaction residue, and modified positions are underlined). These spacing formats with no or minor modifications allow us to test many different sAb designs with a range of CAAP contents (55% to 90%). We designed the CAAP content to be greater than 55%, since the medium value of the natural range (between 37.5% and 75%) of the CAAP content in the 137 CCAAP boxes was 53.8%. For each designated CAAP or non-CAAP, we generally selected the most frequent pairing partner according to the data in FIG. 4B and Table 8.

TABLE 9

| | % CAAP | |
|---|---|---|
| | interactions | |
| | | In |
| | In PPI | non-PPI |
| Interacting Proteins | region | region |
| Saccharomyces cerevisiae GCN4 | 24 | 0 |
| Homodimer [PDB_2ZTA] | | |
| Mus musculus NF-k-B essential modulator | 33 | 0 |
| (NEMO) Homodimer [PDB_4OWF] | | |
| Homo sapiens c-Jun/c-Fos Heterodimer [PDB_1FOS] | 33 | 5 |
| Rattus norvegicus C/EBPA Homodimer | 18 | 7 |
| [PDB_1NWQ] | | |
| Saccharomyces cerevisiae Put3 Homodimer | 25 | 6 |
| [PDB_1AJY] | | |
| Salmonella enterica serovar Typhimurium | 30 | 8 |
| TarH Homodimer [PDB_1VLT] | | |
| Mus musculus E47-NeuroD1 Heterodimer | 26 | 6 |
| [PDB_2QL2] | | |
| Arenicola marina (lugworm) Arenicin-2 | 20 | 0 |
| Homodimer [PDB_2L8X] | | |
| Laticauda semifasciata Erabutoxin | 29 | 0 |
| Homodimer [PDB_1QKD] | | |

### CAAP-Based sAbs can Interact Specifically with Preselected Peptide Sequence in the Target Protein

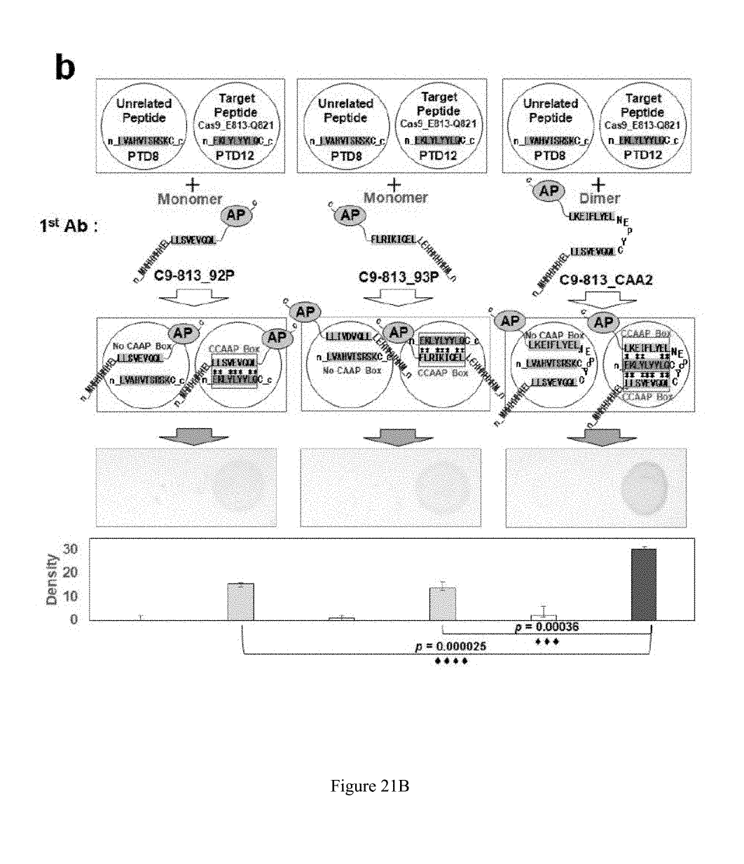

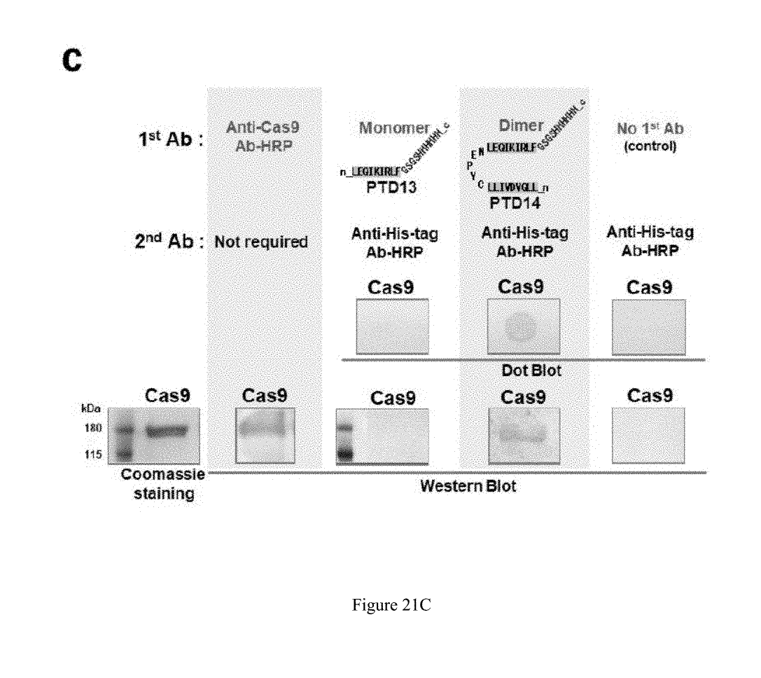

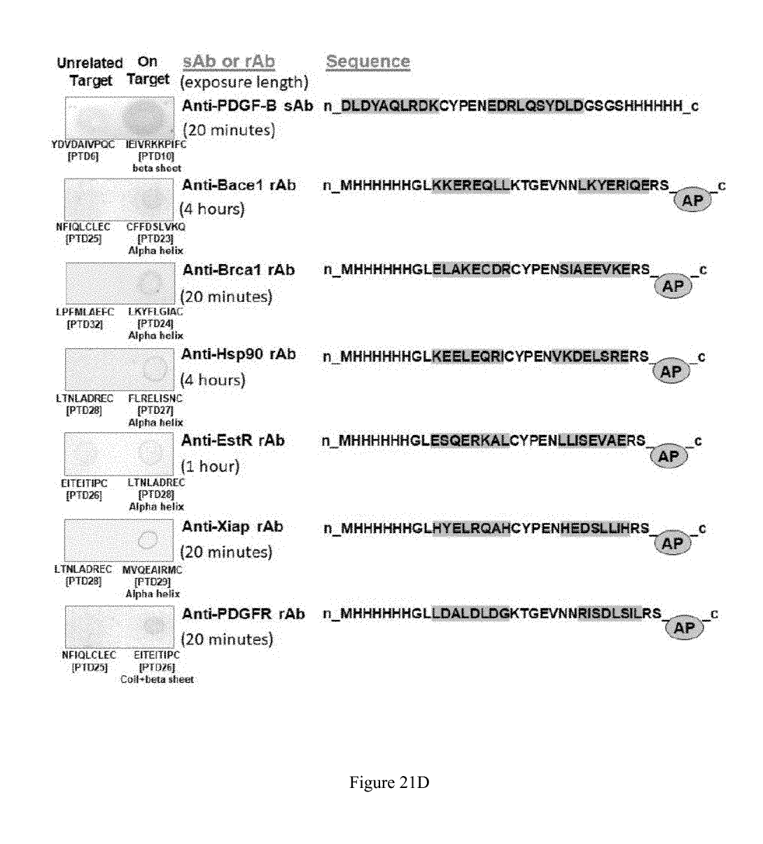

To test the sAb design tool based on the CCAAP principle, we selected a target sequence in the HNH domain of the Staphylococcus pyogenes Cas9 protein [PDB_5B2R]. S. pyogenes CRISPR-Cas9 system has been broadly applied to edit the genome of bacterial and eukaryotic cells. The target sequence for the Cas9 is nEKLYLYYLQc (Helix: E813 to Q821). We designed two different types of synthetic antibody (sAb) molecules, sAb monomer (PTD13, Table 6) and sAb dimer (PTD14, Table 6), to detect the target protein sequences. As shown in the dot blot experiment (FIG. 21A-D), the sAb monomer (PTD13) and sAb dimer (PTD14) could interact with the target peptide (PTD12, Table 6), but no interaction with the control peptide (PTD8, unrelated peptide, Table 6) was detected. No signal was detected from the no peptide control (FIG. 21A). Remarkably, the sAb dimer (PTD14) showed a stronger (two-fold) interaction than that of the sAb monomer PTD13 (FIG. 21A).

To verify these results, we first produced three recombinant antibody (rAb) constructs, C9-813-92P (monomer, parallel), C9-813-93P (monomer, antiparallel), and C9-813-CAA2 (dimer, antiparallel and parallel). As shown in FIG. 21B, we confirmed that the rAb C9-813-CAA2 (dimer, antiparallel and parallel) has stronger (2.5-fold) interaction with the Cas9 target sequence (PTD12) than the rAb C9-813-92P (monomer, parallel) or rAb C9-813-93P (monomer, antiparallel). We confirmed this phenomenon in the two additional cases of detecting alkaline phosphatase (AP) and PDGF-B (FIG. 21D).

Finally, we further examined the performance of the CCAAP oligopeptides to detect the whole Cas9 protein in both non-denatured (dot blot) and denatured (western blot) conditions (FIG. 21C). We used a recombinant Cas9 protein. The purified Cas9 protein is shown in FIG. 21C (Coomassie stain). We used the sAb monomer (PTD13) and sAb dimer (PTD14) as the 1st Ab to detect Cas9 protein. The anti-Cas9 Ab-HRP conjugate was used as positive control 1st Ab in the western blot experiment (FIG. 21C). The sAb dimer (PTD14) was able to detect the Cas9 protein in both the dot blot and western blot, while the monomer and the no peptide (negative control) were unable to detect the Cas9 protein (FIG. 21C). Notably, although the sAb monomer (PTD13) detected the synthetic Cas9 target oligopeptide (PTD12) in the dot blot experiment (FIG. 21C), it failed to detect the whole Cas9 protein (FIG. 21C). This may reflect the molecular weight difference between the target oligopeptide PTD12 (1 kDa,) and Cas9 (160 kDa), which caused the molar ratio (PTD12:Cas9) in the same amount (5 μg) of the samples used for the dot blots to be 160:1.

To generalize the CCAAP principle for protein targeting, we have designed a synthetic antibody (sAb) construct and 6 recombinant antibody (rAb) constructs to detect 7 additional clinically important proteins: Anti-PDGF sAb (PTD18, Table 1) for Human Platelet-Derived Growth Factor B (PDGF-B) [PDB_3MJG]; Anti-Bace1 rAb for Human Bace1 [PDB_4B05]; Anti-Brca1 rAb for Human Brca1 [PDB_3PXE]; Anti-Hsp90 rAb for Human Hsp90 [PDB_2VCI]; Anti-EstR rAb for Human Estrogen Receptor [PDB_1A52]; Anti-Xiap rAb for Human Xiap [PDB_2KNA]; and Anti-PDGFR rAb for PDGF Receptor (PDGFR) [PDB_3MJG] (FIG. 21D). BACE1 is a clinical candidate for the treatment of Alzheimer disease. PDGF-B and PDGFR are known as important targets for antitumor and antiangiogenic therapy. Brca1 and Estrogen receptor proteins are related to breast cancer. Hsp90 chaperone and Xiap are a potential therapeutic target for the treatment of cancer. The dot blot analysis showed that all sAbs and rAbs can specifically interact with their target oligopeptides, while they have no or very weak interaction with the unrelated target oligopeptides, which cannot form a CCAAP box (FIG. 21D). However, the binding affinities of these interactions appeared to be varied as described in FIG. 21D (different exposure time lengths). Although target polypeptide sequence is a key determinant for the binding affinity, we believe that designing an ideal binding sequence for a sAb may reduce the range of variation in the binding strengths.

In the present study, we have developed a novel CCAAP principle and obtained experimental evidence that CCAAP box is a critical driving force for PPI. Therefore, we conclude that the CCAAP concept can be applied to design sAb or rAb that can specifically interact with a preselected oligopeptide sequence (8-10 amino acids) in the target protein.

With respect to the use of substantially any plural and/or singular terms herein, those having skill in the art can translate from the plural to the singular and/or from the singular to plural as is appropriate to the context and/or application. The various singular/plural permutations can be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims can contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims which are incorporated herein by reference.

1. A composition comprising a binding polypeptide configured to interact with a known binding partner wherein said binding polypeptide has a sequence of between 6 and 30 amino acids in length; and
wherein said binding polypeptide sequence is composed by the steps of identifying the sequence of said binding partner; and, identifying 20% or more of the residues in said binding partner sequence; and, for each of the identified residues within the binding partner sequence, selecting the residue at the corresponding position for inclusion in the sequence of said polypeptide sequence as follows:
where the identified residue within the binding partner sequence is Phe, the residue at the corresponding position for inclusion in the sequence of said polypeptide sequence is Lys or Glu;
where the identified residue within the binding partner sequence is Leu, the residue at the corresponding position for inclusion in the sequence of said polypeptide sequence is Gln, Lys, or Glu;
where the identified residue within the binding partner sequence is Ser, the residue at the corresponding position for inclusion in the sequence of said polypeptide sequence is Arg, Gly, Thr, or Ala;
where the identified residue within the binding partner sequence is Thr, the residue at the corresponding position for inclusion in the sequence of said polypeptide sequence is Ser, Gly, Cys, or Arg;
where the identified residue within the binding partner sequence is Tyr, the residue at the corresponding position for inclusion in the sequence of said polypeptide sequence is Ile or Val;
where the identified residue within the binding partner sequence is Cys, the residue at the corresponding position for inclusion in the sequence of said polypeptide sequence is Thr or Ala;
where the identified residue within the binding partner sequence is Trp, the residue at the corresponding position for inclusion in the sequence of said polypeptide sequence is Pro;
where the identified residue within the binding partner sequence is Ile, the residue at the corresponding position for inclusion in the sequence of said polypeptide sequence is Asn, Asp, or Tyr;
where the identified residue within the binding partner sequence is Met, the residue at the corresponding position for inclusion in the sequence of said polypeptide sequence is His;
where the identified residue within the binding partner sequence is Asn, the residue at the corresponding position for inclusion in the sequence of said polypeptide sequence is Ile or Val;
where the identified residue within the binding partner sequence is Lys, the residue at the corresponding position for inclusion in the sequence of said polypeptide sequence is Phe or Leu;
where the identified residue within the binding partner sequence is Arg, the residue at the corresponding position for inclusion in the sequence of said polypeptide sequence is Thr, Ala, Ser, or Pro;
where the identified residue within the binding partner sequence is Pro, the residue at the corresponding position for inclusion in the sequence of said polypeptide sequence is Arg, Gly, or Trp;
where the identified residue within the binding partner sequence is His, the residue at the corresponding position for inclusion in the sequence of said polypeptide sequence is Met or Val;
where the identified residue within the binding partner sequence is Gln, the residue at the corresponding position for inclusion in the sequence of said polypeptide sequence is Leu;
where the identified residue within the binding partner sequence is Val, the residue at the corresponding position for inclusion in the sequence of said polypeptide sequence is Asn, Asp, Tyr, or His;
where the identified residue within the binding partner sequence is Ala, the residue at the corresponding position for inclusion in the sequence of said polypeptide sequence is Ser, Gly, Cys, or Arg;
where the identified residue within the binding partner sequence is Asp, the residue at the corresponding position for inclusion in the sequence of said polypeptide sequence is Ile or Val;
where the identified residue within the binding partner sequence is Glu, the residue at the corresponding position for inclusion in the sequence of said polypeptide sequence is Phe or Leu;
where the identified residue within the binding partner sequence is Gly, the residue at the corresponding position for inclusion in the sequence of said polypeptide sequence is Thr, Ala, Ser, or Pro;
and wherein said binding polypeptide may comprise part of a larger polypeptide.

2. A method of making a polypeptide configured to interact with a known binding partner wherein said binding polypeptide has a sequence of between 6 and 20 amino acids in length; and
wherein said binding polypeptide sequence is assembled by the steps of:
identifying the sequence of said binding partner; and, identifying 20% or more of the residues in said binding partner sequence; and, for each of the identified residues within the binding partner sequence, selecting the corresponding residue for inclusion in the sequence of said binding polypeptide sequence as follows:
where the identified residue within the binding partner sequence is Phe, the residue at the corresponding position for inclusion in the sequence of said polypeptide sequence is Lys or Glu;
where the identified residue within the binding partner sequence is Leu, the residue at the corresponding position for inclusion in the sequence of said polypeptide sequence is Gln, Lys, or Glu;
where the identified residue within the binding partner sequence is Ser, the residue at the corresponding position for inclusion in the sequence of said polypeptide sequence is Arg, Gly, Thr, or Ala;
where the identified residue within the binding partner sequence is Thr, the residue at the corresponding position for inclusion in the sequence of said polypeptide sequence is Ser, Gly, Cys, or Arg;
where the identified residue within the binding partner sequence is Tyr, the residue at the corresponding position for inclusion in the sequence of said polypeptide sequence is Ile or Val;
where the identified residue within the binding partner sequence is Cys, the residue at the corresponding position for inclusion in the sequence of said polypeptide sequence is Thr or Ala;
where the identified residue within the binding partner sequence is Trp, the residue at the corresponding position for inclusion in the sequence of said polypeptide sequence is Pro;
where the identified residue within the binding partner sequence is Ile, the residue at the corresponding position for inclusion in the sequence of said polypeptide sequence is Asn, Asp, or Tyr;
where the identified residue within the binding partner sequence is Met, the residue at the corresponding position for inclusion in the sequence of said polypeptide sequence is His;
where the identified residue within the binding partner sequence is Asn, the residue at the corresponding position for inclusion in the sequence of said polypeptide sequence is Ile or Val;
where the identified residue within the binding partner sequence is Lys, the residue at the corresponding position for inclusion in the sequence of said polypeptide sequence is Phe or Leu;
where the identified residue within the binding partner sequence is Arg, the residue at the corresponding position for inclusion in the sequence of said polypeptide sequence is Thr, Ala, Ser, or Pro;
where the identified residue within the binding partner sequence is Pro, the residue at the corresponding position for inclusion in the sequence of said polypeptide sequence is Arg, Gly, or Trp;
where the identified residue within the binding partner sequence is His, the residue at the corresponding position for inclusion in the sequence of said polypeptide sequence is Met or Val;
where the identified residue within the binding partner sequence is Gln, the residue at the corresponding position for inclusion in the sequence of said polypeptide sequence is Leu;
where the identified residue within the binding partner sequence is Val, the residue at the corresponding position for inclusion in the sequence of said polypeptide sequence is Asn, Asp, Tyr, or His;
where the identified residue within the binding partner sequence is Ala, the residue at the corresponding position for inclusion in the sequence of said polypeptide sequence is Ser, Gly, Cys, or Arg;
where the identified residue within the binding partner sequence is Asp, the residue at the corresponding position for inclusion in the sequence of said polypeptide sequence is Ile or Val;
where the identified residue within the binding partner sequence is Glu, the residue at the corresponding position for inclusion in the sequence of said polypeptide sequence is Phe or Leu;
where the identified residue within the binding partner sequence is Gly, the residue at the corresponding position for inclusion in the sequence of said polypeptide sequence is Thr, Ala, Ser, or Pro;
and wherein said binding polypeptide may comprise part of a larger polypeptide.

3. (canceled)

4. (canceled)

5. (canceled)

6. (canceled)

7. (canceled)

8. The composition of claim 1, wherein the selected corresponding residues for inclusion in the binding polypeptide sequence occur at one of every two positions in the binding polypeptide sequence.

9. The composition of claim 1, wherein the selected corresponding residues for inclusion in the binding polypeptide sequence occur at every other position in the binding polypeptide sequence.

10. The composition of claim 1, wherein the selected corresponding residues for inclusion in the binding polypeptide sequence occur at one of every three positions in the binding polypeptide sequence.

11. The composition of claim 1, wherein the selected corresponding residues for inclusion in the binding polypeptide sequence occur at every third position in the binding polypeptide sequence.

12. The composition of claim 1, wherein the selected corresponding residues for inclusion in the binding polypeptide sequence occur at two of every three positions in the binding polypeptide sequence.

13. A polypeptide made according to the method of claim 2.

14. The polypeptide of claim 1, further comprising a functional moiety.

15. The polypeptide of claim 14 wherein said functional moiety comprises one or more of a polypeptide, a therapeutic molecule, a protein, a nucleic acid, or a diagnostic moiety.

16. The polypeptide of claim 14 wherein said functional moiety comprises one or more of a radiolabel, spin label, affinity tag, or fluorescent label.

17. The polypeptide of claim 14 further comprising a linker.

18. (canceled)

19. The polypeptide of claim 17 wherein said peptide has the sequence GSGS (SEQ ID NO: 1), (G)ₙ (SEQ ID NO: 2), (GS)ₙ (SEQ ID NO: 3), (GGSGG)ₙ (SEQ ID NO: 4), (GGGS)ₙ (SEQ ID NO: 5), CYPEN (SEQ ID NO: 6), or KTGEVNN (SEQ ID NO: 7).

20. A binding polypeptide according to claim 1, wherein said binding polypeptide contains residues configured to interact with a second and optionally a third target protein in addition to the first target protein.

21. A binding polypeptide generated according to claim 2, wherein said binding polypeptide contains residues configured to interact with a second and optionally a third target protein in addition to the first target protein.

22. A fusion polypeptide, wherein said fusion comprises one or more binding polypeptides made according to the method of claim 2.

23. (canceled)

24. The composition of claim 1, wherein said binding polypeptide is incorporated within a fusion polypeptide, and wherein said fusion comprises may further comprise one or more additional binding polypeptides.

25. (canceled)

26. A binding polypeptide according to claim 1, wherein the sequence of said polypeptide comprises one or more of sequence LEQIKRLF (SEQ ID NO: 8), LLQVDVILL (SEQ ID NO: 9), LLQVDVILLCYPENLEQIKIRLF (SEQ ID NO: 10), LLQVDVILLCYPENLEQIKIRLFGSGSHHHHHH (SEQ ID NO: 11), EDRLQSYDLD (SEQ ID NO: 12), EDRLQSYDLDGSGSHHHHHH (SEQ ID NO: 13), ELDKAGFIKRQL (SEQ ID NO: 14), LEERGVKDRQLQ (SEQ ID NO: 15), LEILRAKDLALE (SEQ ID NO: 16), LEQIKIRLF (SEQ ID NO: 17), LSGLNEQRTQ (SEQ ID NO: 18), YDVDAIVPQC (SEQ ID NO: 19), CLTYDSHYLQ (SEQ ID NO: 20), LVAHVTSRKC (SEQ ID NO: 21), EYRLYLRALC (SEQ ID NO: 22), IEIVRKKPIF (SEQ ID NO: 23), IEIVRKKPIFC (SEQ ID NO: 24), CEDRLQSYDLD (SEQ ID NO: 25), EKLYLYYLQ (SEQ ID NO: 26), EKLYLYYLQC (SEQ ID NO: 27), LEQIKIRLFGSGSHHHHHH (SEQ ID NO: 28), LSRAYLSYEGSGSHHHHHH (SEQ ID NO: 29), EYRLYLRALCYPENLSRAYLSYEGSGSHHHHHH (SEQ ID NO: 30), DLDYAQLRDKCYPENEDRLQSYDLDGSGSHHHHHH (SEQ ID NO: 31), GKPIPNPLLGLDST (SEQ ID NO: 32), ELDKAGFIKRQLC (SEQ ID NO: 33), LLQVDVILLHHHHHHLEQIKIRLF (SEQ ID NO: 34), and/or CFFDSLVKQ (SEQ ID NO: 35).

27. A binding polypeptide according to claim 1, or a nucleic acid encoding said binding peptide, wherein the sequence of said polypeptide comprises one or more of the sequences provided in Table 6 or 7.

28. (canceled)

29. A method of making a binding polypeptide configured to interact with a known binding partner wherein said binding polypeptide has a sequence of between 6 and 30 amino acids in length; and
wherein said binding polypeptide sequence is composed by the steps of identifying the sequence of said binding partner; and, identifying 20% or more of the residues in said binding partner sequence; and,
for each of the identified residues within the binding partner sequence, selecting the residue at the corresponding position for inclusion in the sequence of said polypeptide sequence according to the corresponding residues given in Table 10.


SEQUENCE LISTING (582 sequences)

SEQ ID NO: 1 - protein, 4 aa, Artificial Sequence
  Synthetic AgrC1, AgrC2
GSGS

SEQ ID NO: 2 - protein, 1 aa, Artificial Sequence
  Synthetic linker
G

SEQ ID NO: 3 - protein, 2 aa, Artificial Sequence
  Synthetic linker
GS

SEQ ID NO: 4 - protein, 5 aa, Artificial Sequence
  Synthetic linker
GGSGG

SEQ ID NO: 5 - protein, 4 aa, Artificial Sequence
  Synthetic linker
GGGS

SEQ ID NO: 6 - protein, 5 aa, Artificial Sequence
  Synthetic linker
CYPEN

SEQ ID NO: 7 - protein, 7 aa, Artificial Sequence
  Synthetic linker
KTGEVNN

SEQ ID NO: 8 - protein, 8 aa, Artificial Sequence
  Synthetic
LEQIKRLF

SEQ ID NO: 9 - protein, 9 aa, Artificial Sequence
  Synthetic
LLQVDVILL

SEQ ID NO: 10 - protein, 23 aa, Artificial Sequence
  Synthetic
LLQVDVILLCYPENLEQIKIRLF

SEQ ID NO: 11 - protein, 33 aa, Artificial Sequence
  Synthetic
LLQVDVILLCYPENLEQIKIRLFGSGSHHHHHH

SEQ ID NO: 12 - protein, 10 aa, Artificial Sequence
  Synthetic
EDRLQSYDLD

SEQ ID NO: 13 - protein, 20 aa, Artificial Sequence
  Synthetic
EDRLQSYDLDGSGSHHHHHH

SEQ ID NO: 14 - protein, 12 aa, Artificial Sequence
  Synthetic
ELDKAGFIKRQL

SEQ ID NO: 15 - protein, 12 aa, Artificial Sequence
  Synthetic
LEERGVKDRQLQ

SEQ ID NO: 16 - protein, 12 aa, Artificial Sequence
  Synthetic
LEILRAKDLALE

SEQ ID NO: 17 - protein, 9 aa, Artificial Sequence
  Synthetic
LEQIKIRLF

SEQ ID NO: 18 - protein, 10 aa, Artificial Sequence
  Synthetic
LSGLNEQRTQ

SEQ ID NO: 19 - protein, 10 aa, Artificial Sequence
  Synthetic
YDVDAIVPQC

SEQ ID NO: 20 - protein, 10 aa, Artificial Sequence
  Synthetic
CLTYDSHYLQ

SEQ ID NO: 21 - protein, 10 aa, Staphylococcus pyogenes
LVAHVTSRKC

SEQ ID NO: 22 - protein, 10 aa, Artificial Sequence
  Synthetic
EYRLYLRALC

SEQ ID NO: 23 - protein, 10 aa, Staphylococcus pyogenes
IEIVRKKPIF

SEQ ID NO: 24 - protein, 11 aa, Artificial Sequence
  Synthetic
IEIVRKKPIFC

SEQ ID NO: 25 - protein, 11 aa, Artificial Sequence
  Synthetic
CEDRLQSYDLD

SEQ ID NO: 26 - protein, 9 aa, Staphylococcus pyogenes
EKLYLYYLQ

SEQ ID NO: 27 - protein, 10 aa, Artificial Sequence
  Synthetic
EKLYLYYLQC

SEQ ID NO: 28 - protein, 19 aa, Artificial Sequence
  Synthetic
LEQIKIRLFGSGSHHHHHH

SEQ ID NO: 29 - protein, 19 aa, Artificial Sequence
  Synthetic
LSRAYLSYEGSGSHHHHHH

SEQ ID NO: 30 - protein, 33 aa, Artificial Sequence
  Synthetic
EYRLYLRALCYPENLSRAYLSYEGSGSHHHHHH

SEQ ID NO: 31 - protein, 35 aa, Artificial Sequence
  Synthetic
DLDYAQLRDKCYPENEDRLQSYDLDGSGSHHHHHH

SEQ ID NO: 32 - protein, 14 aa, Artificial Sequence
  Synthetic
GKPIPNPLLGLDST

SEQ ID NO: 33 - protein, 13 aa, Artificial Sequence
  Synthetic
ELDKAGFIKRQLC

SEQ ID NO: 34 - protein, 24 aa, Artificial Sequence
  Synthetic
LLQVDVILLHHHHHHLEQIKIRLF

SEQ ID NO: 35 - protein, 9 aa, Artificial Sequence
  Synthetic
CFFDSLVKQ

SEQ ID NO: 36 - DNA, 23 bp, Homo sapiens
ggctactggccttatctcacagg

SEQ ID NO: 37 - DNA, 33 bp, Artificial Sequence
  Primer
taatacgactcactatagggctactggccttat

SEQ ID NO: 38 - DNA, 35 bp, Artificial Sequence
  Primer
ttctagctctaaaacgtgagataaggccagtagcc

SEQ ID NO: 39 - DNA, 21 bp, Artificial Sequence
  Primer
ggaggaatatgtcccagatag

SEQ ID NO: 40 - DNA, 20 bp, Artificial Sequence
  Primer
aaggtttgcttacgatggag

SEQ ID NO: 41 - DNA, 52 bp, Artificial Sequence
  Primer
ccctctagaatagaaggagatttaaatgcaccatcaccaccatcacgagctc

SEQ ID NO: 42 - DNA, 60 bp, Artificial Sequence
  Primer
tcaggatccttacagctgctgaacttcaacgctcagcaggagctcgtgatggtggtgatg

SEQ ID NO: 43 - DNA, 60 bp, Artificial Sequence
  Primer
tcaggatccttaaaacagacggattttaatctgctctaagagctcgtgatggtggtgatg

SEQ ID NO: 44 - DNA, 25 bp, Artificial Sequence
  Primer
ggactttgcgtttctttttcggatc

SEQ ID NO: 45 - DNA, 46 bp, Artificial Sequence
  Primer
agcgttgaagttcagcagctgagatctgtgaaacaaagcactattg

SEQ ID NO: 46 - DNA, 46 bp, Artificial Sequence
  Primer
cagattaaaatccgtctgtttagatctgtgaaacaaagcactattg

SEQ ID NO: 47 - DNA, 64 bp, Artificial Sequence
  Primer
agccggatctcagtggtggtggtggtggtgctcgaggactttgcgtttctttttcggatcctta

SEQ ID NO: 48 - DNA, 18 bp, Artificial Sequence
  Primer
aaaagcaccgactcggtg

SEQ ID NO: 49 - DNA, 143 bp, Artificial Sequence
  Primer
ccctctagaatagaaggagatttaaatgcaccatcaccaccatcacgagctcctgctgagcgttgaagttcagcagctgtaaggatccgaaaaagaaacgcaaagtcctcgagcaccaccaccaccaccactgagatccggct

SEQ ID NO: 50 - DNA, 143 bp, Artificial Sequence
  Primer
ccctctagaatagaaggagatttaaatgcaccatcaccaccatcacgagctcttagagcagattaaaatccgtctgttttaaggatccgaaaaagaaacgcaaagtcctcgagcaccaccaccaccaccactgagatccggct

SEQ ID NO: 51 - DNA, 204 bp, Artificial Sequence
  Primer
agcgttgaagttcagcagctgtgctatccggaaaacctcgaatacctgtttattgaaaaattaagatctgaagccgaaggcaacggcactatagacttcgagctcctgttacaggtggatgtgattctgctcaaaaccggtgaagtcaacaacttagagcagattaaaatccgtctgtttagatctgtgaaacaaagcactatt

SEQ ID NO: 52 - DNA, 1536 bp, Artificial Sequence
  Primer
agcgttgaagttcagcagctgagatctgtgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgcctcaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaagcttccggctctagccatcaccatcaccatcacggttcatctgcggatccgaaaaagaaacgcaaagtcctcgagcaccaccaccaccaccactga

SEQ ID NO: 53 - DNA, 1536 bp, Artificial Sequence
  Primer
cagattaaaatccgtctgtttagatctgtgaaacaaagcactattgcactggcactcttaccgttactgtttacccctgtgacaaaagcccggacaccagaaatgcctgttctggaaaaccgggctgctcagggcgatattactgcacccggcggtgctcgccgtttaacgggtgatcagactgccgctctgcgtgattctcttagcgataaacctgcaaaaaatattattttgctgattggcgatgggatgggggactcggaaattactgccgcacgtaattatgccgaaggtgcgggcggcttttttaaaggtatagatgcctcaccgcttaccgggcaatacactcactatgcgctgaataaaaaaaccggcaaaccggactacgtcaccgactcggctgcatcagcaaccgcctggtcaaccggtgtcaaaacctataacggcgcgctgggcgtcgatattcacgaaaaagatcacccaacgattctggaaatggcaaaagccgcaggtctggcgaccggtaacgtttctaccgcagagttgcaggatgccacgcccgctgcgctggtggcacatgtgacctcgcgcaaatgctacggtccgagcgcgaccagtgaaaaatgtccgggtaacgctctggaaaaaggcggaaaaggatcgattaccgaacagctgcttaacgctcgtgccgacgttacgcttggcggcggcgcaaaaacctttgctgaaacggcaaccgctggtgaatggcagggaaaaacgctgcgtgaacaggcacaggcgcgtggttatcagttggtgagcgatgctgcctcactgaattcggtgacggaagcgaatcagcaaaaacccctgcttggcctgtttgctgacggcaatatgccagtgcgctggctaggaccgaaagcaacgtaccatggcaatatcgataagcccgcagtcacctgtacgccaaatccgcaacgtaatgacagtgtaccaaccctggcgcagatgaccgacaaagccattgaattgttgagtaaaaatgagaaaggctttttcctgcaagttgaaggtgcgtcaatcgataaacaggatcatgctgcgaatccttgtgggcaaattggcgagacggtcgatctcgatgaagccgtacaacgggcgctggaattcgctaaaaaggagggtaacacgctggtcatagtcaccgctgatcacgcccacgccagccagattgttgcgccggataccaaagctccgggcctcacccaggcgctaaataccaaagatggcgcagtgatggtgatgagttacgggaactccgaagaggattcacaagaacataccggcagtcagttgcgtattgcggcgtatggcccgcatgccgccaatgttgttggactgaccgaccagaccgatctcttctacaccatgaaagccgctctggggctgaaagcttccggctctagccatcaccatcaccatcacggttcatctgcggatccgaaaaagaaacgcaaagtcctcgagcaccaccaccaccaccactga

SEQ ID NO: 54 - DNA, 1000 bp, Artificial Sequence
  Primer
ccctctagaatagaaggagatttaaatggataagaaatacagcattggtttggacattggtacgaatagcgttggttgggcagtcattaccgacgagtacaaggtgccgagcaagaagtttaaagtattgggtaacacggaccgtcacagcattaagaaaaacctgattggtgcactgctgtttgacagcggtgaaactgcagaggcgactcgcctgaagcgtaccgcgcgtcgccgctatactcgtcgtaaaaaccgtatctgctatctgcaggagatctttagcaacgagatggcgaaggttgatgacagcttctttcaccgtctggaagaaagcttcctggtcgaagaggacaaaaagcacgagcgccatccgatcttcggcaacattgtggacgaagtggcttatcatgaaaagtatccgaccatttatcatctgcgtaagaagctggttgatagcaccgataaagcggatctgcgtctgatttacctggcactggcccacatgatcaagtttcgcggccactttctgatcgagggtgatctgaatccggacaatagcgacgttgacaagctgttcatccaactggtccaaacgtacaaccagctgttcgaagaaaacccgatcaacgcgagcggtgtggatgcaaaagctattctgagcgcgcgtctgagcaagagccgtcgtttggagaatctgatcgcgcaattgccgggtgagaagaaaaatggcctgttcggtaatctgattgcactgtccctgggcctgacgccgaacttcaaaagcaattttgatctggcagaagatgcgaagctgcaactgagcaaagatacttatgatgacgacctggacaatctgttggcacaaatcggtgaccagtatgcagatctgtttctggcggcaaagaacctgtccgatgcgatcctgctgagcgacattctgcgcgtgaacacggaaattaccaaggctccgctgagcgcgagcatgattaagcgttac

SEQ ID NO: 55 - DNA, 1030 bp, Artificial Sequence
  Primer
ccgctgagcgcgagcatgattaagcgttacgatgagcaccaccaggatctgaccctgctgaaggcgctggtccgtcagcaactgccggaaaagtacaaagagattttctttgaccagagcaagaatggctacgcgggctatatcgatggtggcgctagccaagaagagttctacaagtttatcaagccgattttggagaaaatggatggtaccgaagagttgctggttaaactgaatcgtgaagatctgctgcgtaagcaacgcacctttgataatggcagcattccgcatcaaattcacctgggtgagttgcatgctatcctgcgccgtcaagaggatttctacccgtttctgaaagacaaccgtgagaagatcgagaaaattctgactttccgcatcccgtattacgtcggtccgctggcgcgtggtaacagccgtttcgcatggatgacccgtaagagcgaagaaaccatcaccccatggaacttcgaagaggttgtggataagggtgcatccgcgcaaagcttcatcgagcgtatgacgaattttgacaagaatctgccgaatgaaaaagtgctgccgaagcacagcctgctgtacgaatactttaccgtctataacgagctgaccaaagtcaaatacgtcaccgagggtatgcgtaaaccggcgttcctgagcggcgagcagaagaaggcgattgtcgatctgctgttcaaaacgaatcgtaaagttacggttaagcaactgaaagaggactacttcaagaaaattgaatgtttcgactctgtcgagattagcggtgttgaagatcgcttcaatgcgagcttgggtacctatcatgatctgctgaagatcatcaaagacaaagatttcctggataatgaagagaacgaggacattctggaagatatcgttttgacgctgaccttgttcgaagatcgtgagatgatcgaagaacgcctgaaaacgtatgcgcacctgtttgatgataaagtgatgaaacaactgaagcgtcgccgttataccggtt

SEQ ID NO: 56 - DNA, 1030 bp, Artificial Sequence
  Primer
aacaactgaagcgtcgccgttataccggttggggtcgtctgagccgtaagctgatcaacggcattcgtgataaacagtccggtaagacgatcctggattttctgaaaagcgacggcttcgcaaaccgtaatttcatgcagctgattcacgacgacagcttgaccttcaaagaggacatccagaaagcacaagttagcggtcaaggcgatagcctgcatgagcacattgcaaatttggcgggtagcccagcgatcaagaagggtattctgcagaccgttaaagtggttgatgaactggtgaaagttatgggccgtcacaagcctgaaaacatcgtcattgagatggcgcgtgaaaatcagaccacgcaaaagggccagaagaatagccgtgaacgcatgaaacgtatcgaagagggcattaaagaactgggctcccaaatcctgaaagagcatccggtggagaatactcaactgcagaatgaaaagctgtacctgtactatctgcaaaacggtcgcgatatgtacgtcgaccaggagctggacatcaaccgcctgtccgactatgacgttgatcacattgtcccgcagagcttcctgaaagatgacagcatcgacaacaaggtcctgacccgtagcgataagaatcgcggtaaaagcgataacgtgccaagcgaagaagtggtgaagaagatgaaaaactattggcgtcaactgttgaacgctaaattgattacgcaacgtaagttcgacaacctgaccaaggcggaacgtggtggcctgagcgaactggacaaagcgggtttcatcaagcgccaactggtggaaacccgtcagattacgaaacatgtcgcccaaattctggacagccgtatgaacacgaagtacgatgaaaacgataaactgattcgtgaagtcaaagttatcacgctgaaaagcaagctggtgagcgacttccgtaaggattttcagttttacaaagtccgtgaaatcaacaactaccaccatgcgcacgatgcctatctgaacgctgt

SEQ ID NO: 57 - DNA, 1300 bp, Artificial Sequence
  Primer
ccatgcgcacgatgcctatctgaacgctgtggtgggtaccgcgctgattaagaagtatccgaaactggaaagcgagttcgtgtacggtgattacaaggtttacgatgttcgtaagatgatcgcgaagtccgaacaagaaatcggcaaagcgaccgctaagtatttcttttactccaacattatgaactttttcaaaaccgagatcaccctggcaaacggtgagatccgcaaacgtccgctgatcgagactaatggcgagactggcgaaatcgtgtgggacaaaggtcgtgacttcgccaccgtccgtaaggtattgagcatgccgcaagtcaatattgttaagaaaaccgaagttcaaaccggtggtttcagcaaagagagcattctgcctaagcgcaactccgacaaactgattgcccgtaagaaggattgggacccgaaaaagtatggcggtttcgatagcccaactgtggcatacagcgtgctggtggttgccaaagtggagaaaggtaagtccaagaagctgaaatctgtcaaagagctgctgggcatcaccattatggagcgcagcagctttgagaaaaatccaatcgacttcctggaagcgaagggctacaaagaggtcaagaaagacctgatcatcaagttgccaaagtacagcctgttcgagctggagaatggtcgtaagcgcatgctggcctctgccggtgaactgcaaaagggtaacgaactggcgctgccgtcgaaatacgttaactttctgtacctggcatcccactacgagaaactgaaaggcagccctgaagataacgagcaaaaacaactgtttgttgagcagcacaaacactatctggatgagatcattgaacagattagcgaattcagcaagcgtgtgatcctggcggacgcgaacctggacaaagtcctgtccgcgtacaataaacatcgcgacaaaccgattcgtgagcaggcggaaaacattatccacctgtttaccctgacgaatctgggtgcccctgcggcgtttaagtactttgacactactatcgatcgtaaacgttatacgagcaccaaagaggttctggatgcgaccctgattcaccagagcattaccggcctgtatgaaacgcgtatcgacctgagccaattgggtggtgaccgctctcgtgcagatccgaaaaagaaacgcaaagtcgatccgaagaagaagcgcaaggtggacccgaagaaaaagcgtaaagtcggctctaccggtagccgtggctctggttcgctcgagcaccaccaccaccaccactga

SEQ ID NO: 58 - DNA, 1303 bp, Artificial Sequence
  Primer
ccatgcgcacgatgcctatctgaacgctgtggtgggtaccgcgctgattaagaagtatccgaaactggaaagcgagttcgtgtacggtgattacaaggtttacgatgttcgtaagatgatcgcgaagtccgaacaagaaatcggcaaagcgaccgctaagtatttcttttactccaacattatgaactttttcaaaaccgagatcaccctggcaaacggtgagatccgcaaacgtccgctgatcgagactaatggcgagactggcgaaatcgtgtgggacaaaggtcgtgacttcgccaccgtccgtaaggtattgagcatgccgcaagtcaatattgttaagaaaaccgaagttcaaaccggtggtttcagcaaagagagcattctgcctaagcgcaactccgacaaactgattgcccgtaagaaggattgggacccgaaaaagtatggcggtttcgatagcccaactgtggcatacagcgtgctggtggttgccaaagtggagaaaggtaagtccaagaagctgaaatctgtcaaagagctgctgggcatcaccattatggagcgcagcagctttgagaaaaatccaatcgacttcctggaagcgaagggctacaaagaggtcaagaaagacctgatcatcaagttgccaaagtacagcctgttcgagctggagaatggtcgtaagcgcatgctggcctctgccggtgaactgcaaaagggtaacgaactggcgctgccgtcgaaatacgttaactttctgtacctggcatcccactacgagaaactgaaaggcagccctgaagataacgagcaaaaacaactgtttgttgagcagcacaaacactatctggatgagatcattgaacagattagcgaattcagcaagcgtgtgatcctggcggacgcgaacctggacaaagtcctgtccgcgtacaataaacatcgcgacaaaccgattcgtgagcaggcggaaaacattatccacctgtttaccctgacgaatctgggtgcccctgcggcgtttaagtactttgacactactatcgatcgtaaacgttatacgagcaccaaagaggttctggatgcgaccctgattcaccagagcattaccggcctgtatgaaacgcgtatcgacctgagccaattgggtggtgaccgctctcgtgcagatccgaaaaagaaacgcaaagtcgatccgaagaagaagcgcaaggtggacccgaagaaaaagcgtaaagtcggctctaccggtagccgtggctctggttcgtaactcgagcaccaccaccaccaccactga

SEQ ID NO: 59 - DNA, 118 bp, Artificial Sequence
  Primer
taatacgactcactatagggctactggccttatctcacgttttagagctagaaatagcaagttaaaataaggctagtccgttatcaacttgaaaaagtggcaccgagtcggtgctttt

SEQ ID NO: 60 - DNA, 258 bp, Artificial Sequence
  Primer
gcggataacaattcccctctagaatagaaggagatttaaatgagccgtaaagaagcacgcgagctctgttacccggagaatggtctggaagcactgattagatctggaggtggaggttcaggtggaggtggatccggtggtggaggatcatattatctgcgtaaacgtattctgtgctacccggaaaatcaggttctggaacgtagcaatgaaggtagtggtagcaagcttctcgagcaccaccaccaccaccactga

SEQ ID NO: 61 - DNA, 510 bp, Artificial Sequence
  Primer
ggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggacggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacggccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaacctt

SEQ ID NO: 62 - protein, 8 aa, Homo sapiens
KAKERLEA

SEQ ID NO: 63 - protein, 8 aa, Homo sapiens
FHKLTHQR

SEQ ID NO: 64 - protein, 8 aa, Homo sapiens
ERQQLVET

SEQ ID NO: 65 - protein, 8 aa, Homo sapiens
LSLSQNMR

SEQ ID NO: 66 - protein, 8 aa, Homo sapiens
ELSAATHL

SEQ ID NO: 67 - protein, 8 aa, Homo sapiens
LHTAASLE

SEQ ID NO: 68 - protein, 8 aa, Homo sapiens
TSVQNVRR

SEQ ID NO: 69 - protein, 8 aa, Homo sapiens
RSTYVDET

SEQ ID NO: 70 - protein, 10 aa, Enterobacter sp. RFL1396
FEMLIKEILK

SEQ ID NO: 71 - protein, 10 aa, Enterobacter sp. RFL1396
KLIEKILMEF

SEQ ID NO: 72 - protein, 12 aa, Helicobacter pylori 26695
IGGTASLITASQ

SEQ ID NO: 73 - protein, 12 aa, Helicobacter pylori 26695
YQRKSQELSREL

SEQ ID NO: 74 - protein, 16 aa, Helicobacter pylori 26695
LEELDALERSLEQSKR

SEQ ID NO: 75 - protein, 16 aa, Helicobacter pylori 26695
KLSEVLTQSATILSAT

SEQ ID NO: 76 - protein, 8 aa, Cyanothece sp. (strain ATCC 51142)
LKKKVRKL

SEQ ID NO: 77 - protein, 8 aa, Cyanothece sp. (strain ATCC 51142)
KKKLQDLE

SEQ ID NO: 78 - protein, 8 aa, Mycobacterium tuberculosis
QSSLERAN

SEQ ID NO: 79 - protein, 8 aa, Mycobacterium tuberculosis
NARELSSQ

SEQ ID NO: 80 - protein, 8 aa, Allochromatium vinosum
AGLSPEEQ

SEQ ID NO: 81 - protein, 8 aa, Allochromatium vinosum
GAQRTEIQ

SEQ ID NO: 82 - protein, 8 aa, Allochromatium vinosum
IAAIANSG

SEQ ID NO: 83 - protein, 8 aa, Allochromatium vinosum
MGSNAIAA

SEQ ID NO: 84 - protein, 10 aa, Homo sapiens
LREHFEKLEK

SEQ ID NO: 85 - protein, 10 aa, Homo sapiens
KELKEFHERL

SEQ ID NO: 86 - protein, 8 aa, Homo sapiens
KRCSCSSL

SEQ ID NO: 87 - protein, 8 aa, Homo sapiens
LSSCSCRK

SEQ ID NO: 88 - protein, 8 aa, Shewanella sp. SIB1
SYGVGRQG

SEQ ID NO: 89 - protein, 8 aa, Shewanella sp. SIB1
RRSIETFA

SEQ ID NO: 90 - protein, 12 aa, Homo sapiens
LEKEKSEFKLEL

SEQ ID NO: 91 - protein, 12 aa, Homo sapiens
KLEKEKSEFKLE

SEQ ID NO: 92 - protein, 12 aa, Hepatitis delta virus
KLEELERDLRKL

SEQ ID NO: 93 - protein, 12 aa, Hepatitis delta virus
LKRLDRELEELK

SEQ ID NO: 94 - protein, 8 aa, Haemophilus influenzae
ASNLLTTS

SEQ ID NO: 95 - protein, 8 aa, Haemophilus influenzae
STTLLNSA

SEQ ID NO: 96 - protein, 8 aa, Haemophilus influenzae
SLINAVKT

SEQ ID NO: 97 - protein, 8 aa, Haemophilus influenzae
TKVANILS

SEQ ID NO: 98 - protein, 8 aa, Helicobacter pylori
LERFKELL

SEQ ID NO: 99 - protein, 8 aa, Helicobacter pylori
RLLEKFRE

SEQ ID NO: 100 - protein, 32 aa, Helicobacter pylori
DKFSEVLDNLKSTFNEFDEAAQEQIAWLKERI

SEQ ID NO: 101 - protein, 32 aa, Helicobacter pylori
IREKLWAIQEQAAEDFENFTSKLNDLVESFKD

SEQ ID NO: 102 - protein, 9 aa, Bos taurus
QSIKKLKQS

SEQ ID NO: 103 - protein, 9 aa, Bos taurus
LAALQEKAR

SEQ ID NO: 104 - protein, 16 aa, Homo sapiens
LSGEQEVLRGELEAAK

SEQ ID NO: 105 - protein, 16 aa, Homo sapiens
KAAELEGRLVEQEGSL

SEQ ID NO: 106 - protein, 8 aa, Bacteriophage Lambda
MEQRITLK

SEQ ID NO: 107 - protein, 8 aa, Bacteriophage Lambda
DKLTIRQE

SEQ ID NO: 108 - protein, 9 aa, HIV-1 HXB3
RLIKFLYQS

SEQ ID NO: 109 - protein, 9 aa, HIV-1 HXB3
SQYLFKILR

SEQ ID NO: 110 - protein, 11 aa, HIV-1 HXB3
SERIRSTYLGR

SEQ ID NO: 111 - protein, 11 aa, HIV-1 HXB3
RGLYTSRIRES

SEQ ID NO: 112 - protein, 8 aa, Escherichia coli
FIRSQTLT

SEQ ID NO: 113 - protein, 8 aa, Escherichia coli
ELLTLTQS

SEQ ID NO: 114 - protein, 10 aa, Escherichia coli
ESLHDHADEL

SEQ ID NO: 115 - protein, 10 aa, Escherichia coli
FRALCSRYLE

SEQ ID NO: 116 - protein, 9 aa, Homo sapiens
SLSQASADL

SEQ ID NO: 117 - protein, 9 aa, Homo sapiens
RKTLSQEIE

SEQ ID NO: 118 - protein, 8 aa, Homo sapiens
QSTIDLKN

SEQ ID NO: 119 - protein, 8 aa, Homo sapiens
LRGICQKL

SEQ ID NO: 120 - protein, 19 aa, Homo sapiens
KSYVHSALKIFKTAEECRL

SEQ ID NO: 121 - protein, 19 aa, Homo sapiens
LRCEEATKFIKLASHVYSK

SEQ ID NO: 122 - protein, 8 aa, Homo sapiens
YVLYMKYV

SEQ ID NO: 123 - protein, 8 aa, Homo sapiens
VYKMYLVY

SEQ ID NO: 124 - protein, 8 aa, Homo sapiens
RCVIFITF

SEQ ID NO: 125 - protein, 8 aa, Homo sapiens
ITYTKIRS

SEQ ID NO: 126 - protein, 8 aa, Homo sapiens
GSMSVTGI

SEQ ID NO: 127 - protein, 8 aa, Homo sapiens
PKFTYSII

SEQ ID NO: 128 - protein, 10 aa, Caenorhabditis elegans
QRILELMEHV

SEQ ID NO: 129 - protein, 10 aa, Homo sapiens
LIRKLEKADN

SEQ ID NO: 130 - protein, 8 aa, Caenorhabditis elegans
ASLQQVLQ

SEQ ID NO: 131 - protein, 8 aa, Homo sapiens
SIEELVEK

SEQ ID NO: 132 - protein, 8 aa, Saccharomyces cerevisiae
IQELRKLL

SEQ ID NO: 133 - protein, 8 aa, Saccharomyces cerevisiae
DILKNIQR

SEQ ID NO: 134 - protein, 10 aa, Homo sapiens
LQKRLLALDP

SEQ ID NO: 135 - protein, 10 aa, Homo sapiens
ERLAEELKQR

SEQ ID NO: 136 - protein, 8 aa, Homo sapiens
VLDRLKMK

SEQ ID NO: 137 - protein, 8 aa, Mus musculus
NQVLQLLL

SEQ ID NO: 138 - protein, 8 aa, Homo sapiens
LSMFYETL

SEQ ID NO: 139 - protein, 8 aa, Mus musculus
QIHKLSSF

SEQ ID NO: 140 - protein, 8 aa, Homo sapiens
LFSKELRC

SEQ ID NO: 141 - protein, 8 aa, Homo sapiens
EYRNLQEE

SEQ ID NO: 142 - protein, 14 aa, Homo sapiens
LEDLVIEFITEMTH

SEQ ID NO: 143 - protein, 14 aa, Homo sapiens
EVVEGVFVKSIGSM

SEQ ID NO: 144 - protein, 14 aa, Schizosaccharomyces pombe
VQKHIDLLHTYNEI

SEQ ID NO: 145 - protein, 14 aa, Schizosaccharomyces pombe
HLLDIHKQVTQKAD

SEQ ID NO: 146 - protein, 12 aa, Schizosaccharomyces pombe
EQQKEQLESSLQ

SEQ ID NO: 147 - protein, 12 aa, Schizosaccharomyces pombe
LKALADQLSSEL

SEQ ID NO: 148 - protein, 8 aa, Arenicola marina
VYAYVRIR

SEQ ID NO: 149 - protein, 8 aa, Arenicola marina
RWCVYAYV

SEQ ID NO: 150 - protein, 15 aa, Homo sapiens
EALEKSEARRKELEE

SEQ ID NO: 151 - protein, 15 aa, Homo sapiens
LKEALEKSEARRKEL

SEQ ID NO: 152 - protein, 10 aa, Homo sapiens
EKNDLQLQVQ

SEQ ID NO: 153 - protein, 10 aa, Homo sapiens
LLQEKNDLQL

SEQ ID NO: 154 - protein, 10 aa, Homo sapiens
ELKRDIDDLE

SEQ ID NO: 155 - protein, 10 aa, Homo sapiens
LKRDIDDLEL

SEQ ID NO: 156 - protein, 8 aa, Mus musculus
LKEKLEES

SEQ ID NO: 157 - protein, 8 aa, Mus musculus
ELKEKLEE

SEQ ID NO: 158 - protein, 9 aa, Homo sapiens
LEDLKQQLQ

SEQ ID NO: 159 - protein, 9 aa, Homo sapiens
QLEDLKQQL

SEQ ID NO: 160 - protein, 10 aa, Homo sapiens
LLQEQLEQLQ

SEQ ID NO: 161 - protein, 10 aa, Homo sapiens
ELLQEQLEQL

SEQ ID NO: 162 - protein, 8 aa, Homo sapiens
AYFAMVKR

SEQ ID NO: 163 - protein, 8 aa, Homo sapiens
GEAMAYFA

SEQ ID NO: 164 - protein, 8 aa, Homo sapiens
HLEHDLVH

SEQ ID NO: 165 - protein, 8 aa, Homo sapiens
VQSHILHL

SEQ ID NO: 166 - protein, 8 aa, Homo sapiens
LEKRLSEK

SEQ ID NO: 167 - protein, 8 aa, Homo sapiens
KELEKRLS

SEQ ID NO: 168 - protein, 12 aa, Drosophila melanogaster
EEGQYVVNEYSR

SEQ ID NO: 169 - protein, 12 aa, Drosophila melanogaster
LMPLMYVILKDA

SEQ ID NO: 170 - protein, 8 aa, Mus musculus
VEAAVNRL

SEQ ID NO: 171 - protein, 8 aa, Mus musculus
HFFRELAE

SEQ ID NO: 172 - protein, 8 aa, Homo sapiens
AGSVYAGI

SEQ ID NO: 173 - protein, 8 aa, Homo sapiens
EAGSVYAG

SEQ ID NO: 174 - protein, 8 aa, Shewanella sp. SIB1
GVGRQGEQ

SEQ ID NO: 175 - protein, 8 aa, Shewanella sp. SIB1
AGLADAFA

SEQ ID NO: 176 - protein, 8 aa, Saccharomyces cerevisiae
RLERLEQL

SEQ ID NO: 177 - protein, 8 aa, Saccharomyces cerevisiae
SRLERLEQ

SEQ ID NO: 178 - protein, 15 aa, Saccharomyces cerevisiae
RRSRARKLQRMKQLE

SEQ ID NO: 179 - protein, 15 aa, Saccharomyces cerevisiae
ARRSRARKLQRMKQL

SEQ ID NO: 180 - protein, 8 aa, Caenorhabditis elegans
ADLVKEKK

SEQ ID NO: 181 - protein, 8 aa, Caenorhabditis elegans
NVERLLDD

SEQ ID NO: 182 - protein, 8 aa, Caenorhabditis elegans
SNVERLLD

SEQ ID NO: 183 - protein, 8 aa, Caenorhabditis elegans
LADLVKEK

SEQ ID NO: 184 - protein, 8 aa, Methanobacterium fervidus
SDDARIAL

SEQ ID NO: 185 - protein, 8 aa, Methanobacterium fervidus
RIIKNAGA

SEQ ID NO: 186 - protein, 8 aa, Mus musculus
LSQLQTEL

SEQ ID NO: 187 - protein, 8 aa, Mus musculus
KLSQLQTE

SEQ ID NO: 188 - protein, 8 aa, Mus musculus
LSQLQTEL

SEQ ID NO: 189 - protein, 8 aa, Mus musculus
EALIQALG

SEQ ID NO: 190 - protein, 8 aa, Mus musculus
LNKLLKQN

SEQ ID NO: 191 - protein, 8 aa, Mus musculus
ERLNKLLK

SEQ ID NO: 192 - protein, 8 aa, Arabidopsis thaliana
SAYLSELE

SEQ ID NO: 193 - protein, 8 aa, Arabidopsis thaliana
GSAYLSEL

SEQ ID NO: 194 - protein, 8 aa, Homo sapiens
ALSEMIQF

SEQ ID NO: 195 - protein, 8 aa, Homo sapiens
SKAVEQVK

SEQ ID NO: 196 - protein, 20 aa, Homo sapiens
LARERDTSRRLLAEKEREMA

SEQ ID NO: 197 - protein, 20 aa, Homo sapiens
EDSLARERDTSRRLLAEKER

SEQ ID NO: 198 - protein, 8 aa, Homo sapiens
DSFHSLRD

SEQ ID NO: 199 - protein, 8 aa, Homo sapiens
IQYMRRKV

SEQ ID NO: 200 - protein, 8 aa, Homo sapiens
RALEGSGC

SEQ ID NO: 201 - protein, 8 aa, Homo sapiens
VRALEGSG

SEQ ID NO: 202 - protein, 8 aa, Bos taurus
KQVEEILR

SEQ ID NO: 203 - protein, 8 aa, Bos taurus
LQQLRDEE

SEQ ID NO: 204 - protein, 9 aa, Bos taurus
LQKLQQLRD

SEQ ID NO: 205 - protein, 9 aa, Bos taurus
EILRLEKEI

SEQ ID NO: 206 - protein, 8 aa, Mus musculus
LRQQLQQA

SEQ ID NO: 207 - protein, 8 aa, Mus musculus
EDLRQQLQ

SEQ ID NO: 208 - protein, 11 aa, Mus musculus
QEQLEQLQREF

SEQ ID NO: 209 - protein, 11 aa, Mus musculus
LQEQLEQLQRE

SEQ ID NO: 210 - protein, 9 aa, Simian rotavirus A/SA11
LQVYNNKLE

SEQ ID NO: 211 - protein, 9 aa, Simian rotavirus A/SA11
ELQVYNNKL

SEQ ID NO: 212 - protein, 8 aa, Simian rotavirus A/SA12
NKIGSLTS

SEQ ID NO: 213 - protein, 8 aa, Simian rotavirus A/SA12
AFDDLESV

SEQ ID NO: 214 - protein, 10 aa, Synthetic construct
ELEVARLKKL

SEQ ID NO: 215 - protein, 10 aa, Synthetic construct
LELEVARLKK

SEQ ID NO: 216 - protein, 9 aa, Homo sapiens
LKRKLHKLQ

SEQ ID NO: 217 - protein, 9 aa, Homo sapiens
ELKRKLHKL

SEQ ID NO: 218 - protein, 20 aa, Homo sapiens
DELELELDQKDELIQLQNEL

SEQ ID NO: 219 - protein, 20 aa, Homo sapiens
IDELELELDQKDELIQLQNE

SEQ ID NO: 220 - protein, 8 aa, Saccharomyces cerevisiae
LQQLQKDL

SEQ ID NO: 221 - protein, 8 aa, Saccharomyces cerevisiae
KYLQQLQK

SEQ ID NO: 222 - protein, 11 aa, Mus musculus
LDEEISRVRKD

SEQ ID NO: 223 - protein, 11 aa, Mus musculus
ERLLDEEISRV

SEQ ID NO: 224 - protein, 15 aa, Saccharomyces cerevisiae
GADSLNVAMDCISEA

SEQ ID NO: 225 - protein, 15 aa, Saccharomyces cerevisiae
ASKEEIAALIVNYFS

SEQ ID NO: 226 - protein, 8 aa, S. Typhimurium
LRQQQSEL

SEQ ID NO: 227 - protein, 8 aa, S. Typhimurium
ISNELRQQ

SEQ ID NO: 228 - protein, 18 aa, Staphylococcus aureus aureus MW2
EVLDTQFGLQKEVDFAVK

SEQ ID NO: 229 - protein, 18 aa, Staphylococcus aureus aureus MW2
LYEEVLDTQFGLQKEVDF

SEQ ID NO: 230 - protein, 8 aa, Homo sapiens
KAEELKAE

SEQ ID NO: 231 - protein, 8 aa, Homo sapiens
SRLATLRS

SEQ ID NO: 232 - protein, 12 aa, Mus musculus
LEKKNEALKERA

SEQ ID NO: 233 - protein, 12 aa, Mus musculus
ERLQKKVEQLSR

SEQ ID NO: 234 - protein, 9 aa, Mus musculus
LEDEKSALQ

SEQ ID NO: 235 - protein, 9 aa, Mus musculus
QLIQQVEQL

SEQ ID NO: 236 - protein, 8 aa, Homo sapiens
LKAQNSEL

SEQ ID NO: 237 - protein, 8 aa, Homo sapiens
EDEKSALQ

SEQ ID NO: 238 - protein, 8 aa, Homo sapiens
VAQLKQKV

SEQ ID NO: 239 - protein, 8 aa, Homo sapiens
EKLEFILA

SEQ ID NO: 240 - protein, 8 aa, Homo sapiens
AQECQNLE

SEQ ID NO: 241 - protein, 8 aa, Homo sapiens
RLEGLTQD

SEQ ID NO: 242 - protein, 10 aa, Mus musculus
LILQQAVQVI

SEQ ID NO: 243 - protein, 10 aa, Mus musculus
KIETLRLAKN

SEQ ID NO: 244 - protein, 8 aa, Homo sapiens
GCPAEQRA

SEQ ID NO: 245 - protein, 8 aa, Homo sapiens
TNGPKIPS

SEQ ID NO: 246 - protein, 12 aa, Homo sapiens
EERVSELRHQLQ

SEQ ID NO: 247 - protein, 12 aa, Homo sapiens
LDKDLEEVTMQL

SEQ ID NO: 248 - protein, 8 aa, Caenorhabditis elegans
REVYETVY

SEQ ID NO: 249 - protein, 8 aa, Homo sapiens
THDVVAHE

SEQ ID NO: 250 - protein, 8 aa, Saccharomyces cerevisiae
LLEEQLEY

SEQ ID NO: 251 - protein, 8 aa, Saccharomyces cerevisiae
QKKLVEVE

SEQ ID NO: 252 - protein, 8 aa, Homo sapiens
LRKRREQL

SEQ ID NO: 253 - protein, 8 aa, Homo sapiens
KRQNALLE

SEQ ID NO: 254 - protein, 8 aa, Homo sapiens
LSKNEILR

SEQ ID NO: 255 - protein, 8 aa, Homo sapiens
KLLILQQA

SEQ ID NO: 256 - protein, 8 aa, Escherichia coli
ARANQRAD

SEQ ID NO: 257 - protein, 8 aa, Escherichia coli
AARANQRA

SEQ ID NO: 258 - protein, 8 aa, Rattus norvegicus
VLELTSDN

SEQ ID NO: 259 - protein, 8 aa, Rattus norvegicus
KVLELTSD

SEQ ID NO: 260 - protein, 8 aa, Rattus norvegicus
QLSRELDT

SEQ ID NO: 261 - protein, 8 aa, Rattus norvegicus
EQLSRELD

SEQ ID NO: 262 - protein, 10 aa, Homo sapiens
KAQNSELAST

SEQ ID NO: 263 - protein, 10 aa, Homo sapiens
LKAQNSELAS

SEQ ID NO: 264 - protein, 8 aa, Homo sapiens
KLTVEDLE

SEQ ID NO: 265 - protein, 8 aa, Homo sapiens
LKLTVEDL

SEQ ID NO: 266 - protein, 8 aa, Homo sapiens
LQRIVDIL

SEQ ID NO: 267 - protein, 8 aa, Homo sapiens
VLQRIVDI

SEQ ID NO: 268 - protein, 11 aa, Homo sapiens
EALKENEKLHK

SEQ ID NO: 269 - protein, 11 aa, Homo sapiens
LYEALKENEKL

SEQ ID NO: 270 - protein, 10 aa, Escherichia coli
KDDFARFNQR

SEQ ID NO: 271 - protein, 10 aa, Escherichia coli
FNAFRSDFQA

SEQ ID NO: 272 - protein, 8 aa, Homo sapiens
EIRAAFLE

SEQ ID NO: 273 - protein, 8 aa, Homo sapiens
LEIRAAFL

SEQ ID NO: 274 - protein, 57 aa, Homo sapiens
RKRMRNRIAASKSRKRKLERIARLEEKVKTLKAQNSELASTANMLREQVAQLKQKVM

SEQ ID NO: 275 - protein, 60 aa, Homo sapiens
KRRIRRERNKMAAAKSRNRRRELTDTLQAETDQLEDEKSALQTEIANLLKEKEKLEFILA

SEQ ID NO: 276 - protein, 88 aa, Homo sapiens
GHMNVKRRTHNVLERQRRNELKRSFFALRDQIPELENNEKAPKVVILKKATAYILSVQAEEQKLISEEDLLRKRREQLKHKLEQLGGC

SEQ ID NO: 277 - protein, 83 aa, Homo sapiens
DKRAHHNALERKRRDHIKDSFHSLRDSVPSLQGEKASRAQILDKATEYIQYMRRKNHTHQQDIDDLKRQNALLEQQVRALGGC

SEQ ID NO: 278 - protein, 42 aa, A. thaliana Hy5
GSAYLSELENRVKDLENKNSELEERLSTLQNENQMLRHILKN

SEQ ID NO: 279 - protein, 53 aa, Yeast GCN4
ALKRARNTEAARRSRARKLQRMKQLEDKVEELLSKNYHLENEVARLKKLVGER

SEQ ID NO: 280 - protein, 80 aa, S. aureus
QATKNAALKQLTKDADEILHLIKVQLDNCPLYEEVLDTQFGLQKEVDFAVKLGLVDREDGKQILRLEKELSKLHEAFTLV

SEQ ID NO: 281 - protein, 22 aa, S. aureus
LYEEVLDTQFGLQKEVDFAVKL

SEQ ID NO: 282 - protein, 59 aa, D. melanogaster
QDVFLDYCQKLLEKFRYPWELMPLMYVILKDADANIEEASRRIEEGQYVVNEYSRQHNL

SEQ ID NO: 283 - protein, 22 aa, D. melanogaster
EASRRIEEGQYVVNEYSRQHNL

SEQ ID NO: 284 - protein, 15 aa, D. melanogaster
ELMPLMYVILKDADA

SEQ ID NO: 285 - protein, 58 aa, Homo sapiens
VLQVLDRLKMKLQEKGDTSQNEKLSMFYETLKSPLFNQILTLQQSIKQLKGQLNHILE

SEQ ID NO: 286 - protein, 51 aa, Mus musculus
QDPDVEDLFSSLKHIQHTLVDSQSQEDISLLLQLVQNRDFQNAFKIHNAVT

SEQ ID NO: 287 - protein, 29 aa, Homo sapiens
VLQVLDRLKMKLQEKQNEKLSMFYETLKS

SEQ ID NO: 288 - protein, 33 aa, Mus musculus
SQSQEDISLLLQLVQNQDPDVEDLFSSLKHIQH

SEQ ID NO: 289 - protein, 10 aa, Artificial Sequence
  Synthetic
GSGSHHHHHH

SEQ ID NO: 290 - protein, 18 aa, Artificial Sequence
  Synthetic
MHHHHHHELLLSVEVQQL

SEQ ID NO: 291 - protein, 18 aa, Artificial Sequence
  Synthetic
MHHHHHHELLEQIKIRLF

SEQ ID NO: 292 - protein, 18 aa, Artificial Sequence
  Synthetic
MHHHHHHELLLQVDVILL

SEQ ID NO: 293 - protein, 32 aa, Artificial Sequence
  Synthetic
MHHHHHHELLLSVEVQQLCYPENLEYLFIEKL

SEQ ID NO: 294 - protein, 68 aa, Artificial Sequence
  Synthetic
MHHHHHHLLSVEVQQLCYPENLEYLFIEKLRSEAEGNGTIDFELLQVDVILLKTGEVNNLEQIKIRLF

SEQ ID NO: 295 - protein, 19 aa, Artificial Sequence
  Synthetic
LSRAYLSYEGSGSHHHHHH

SEQ ID NO: 296 - DNA, 33 bp, Artificial Sequence
  Synthetic
gggctggctactggccttatctcacaggtaaaa

SEQ ID NO: 297 - protein, 8 aa, Artificial Sequence
  Synthetic
LVRSFANS

SEQ ID NO: 298 - protein, 8 aa, Artificial Sequence
  Synthetic
PLLGLDST

SEQ ID NO: 299 - protein, 69 aa, Artificial Sequence
  Synthetic
MSRKEARELCYPENGLEALIRSGGGSGGGSGGGSYYLRKRILCYPENQVLERSNEGSGSKLLEHHHHHH

SEQ ID NO: 300 - protein, 9 aa, Artificial Sequence
  Synthetic
LKYFLGIAC

SEQ ID NO: 301 - protein, 9 aa, Artificial Sequence
  Synthetic
NFIQLCLEC

SEQ ID NO: 302 - protein, 9 aa, Artificial Sequence
  Synthetic
EITEITIPC

SEQ ID NO: 303 - protein, 9 aa, Artificial Sequence
  Synthetic
FLRELISNC

SEQ ID NO: 304 - protein, 9 aa, Artificial Sequence
  Synthetic
LTNLADREC

SEQ ID NO: 305 - protein, 9 aa, Artificial Sequence
  Synthetic
MVQEAIRMC

SEQ ID NO: 306 - protein, 9 aa, Artificial Sequence
  Synthetic
LPFMLAEFC

SEQ ID NO: 307 - DNA, 130 bp, Artificial Sequence
  Synthetic
ccctctagaatagaaggagatttaaatgcaccatcaccaccatcacgagctcaaaaaagaacgtgaacagctgctgaaaaccggtgaagtcaacaacctgaaatatgaacgtattcaagagagatctgtg

SEQ ID NO: 308 - DNA, 124 bp, Artificial Sequence
  Synthetic
ccctctagaatagaaggagatttaaatgcaccatcaccaccatcacgagctcgaactggccaaagaatgtgatcgttgctatccggaaaacagcattgcagaagaagtgaaagaaagatctgtg

SEQ ID NO: 309 - DNA, 124 bp, Artificial Sequence
  Synthetic
ccctctagaatagaaggagatttaaatgcaccatcaccaccatcacgagctccattatgaactgcgtcaggcacattgctatccggaaaaccatgaagatagcctgctgattcatagatctgtg

SEQ ID NO: 310 - DNA, 124 bp, Artificial Sequence
  Synthetic
ccctctagaatagaaggagatttaaatgcaccatcaccaccatcacgagctcaaagaagaactggaacagcgtatctgctatccggaaaacgtcaaagatgaactgagccgtgaaagatctgtg

SEQ ID NO: 311 - DNA, 124 bp, Artificial Sequence
  Synthetic
ccctctagaatagaaggagatttaaatgcaccatcaccaccatcacgagctcgaaagccaagaacgtaaagcactgtgctatccggaaaacctgttaattagcgaagttgccgaaagatctgtg

SEQ ID NO: 312 - DNA, 130 bp, Artificial Sequence
  Synthetic
ccctctagaatagaaggagatttaaatgcaccatcaccaccatcacgagctcctggatgcactggatctggatggtaaaaccggtgaagtcaacaaccgtattagcgatctgagcattctgagatctgtg

SEQ ID NO: 313 - protein, 5 aa, Rattus norvegicus
  CD2 chain A_beta sheet 5
TYNVT

SEQ ID NO: 314 - protein, 5 aa, Rattus norvegicus
  CD2 chain B_beta sheet 1
GREWR

SEQ ID NO: 315 - protein, 12 aa, Hepatitis delta virus
  HDAg chain A_helix 1
LEELERDLRKLK

SEQ ID NO: 316 - protein, 12 aa, Hepatitis delta virus
  HDAg chain B_helix 1
KLKRLDRELEEL

SEQ ID NO: 317 - protein, 18 aa, Saccharomyces cerevisiae
  Put3 chain A_helix Put3 chain B_helix
LEPSKKIVVSTKYLQQLQ

SEQ ID NO: 318 - protein, 18 aa, Saccharomyces cerevisiae
  Put3 chain A_helix Put3 chain B_helix
EPSKKIVVSTKYLQQLQK

SEQ ID NO: 319 - protein, 8 aa, Allochromatium vinosum
  Cytochrome C chain A_helix 1
LSPEEQIE

SEQ ID NO: 320 - protein, 8 aa, Allochromatium vinosum
  Cytochrome C chain B_helix 1
KGMNWGMF

SEQ ID NO: 321 - protein, 8 aa, Homo sapiens
  TAFII-18 chain A_helix 1
LFSKELRC

SEQ ID NO: 322 - protein, 8 aa, Homo sapiens
  TAFII-28 chain B_helix 1
EYRNLQEE

SEQ ID NO: 323 - protein, 14 aa, Homo sapiens
  TAFII-18 chain A_helix 2
LEDLVIEFITEMTH

SEQ ID NO: 324 - protein, 14 aa, Homo sapiens
  TAFII-28 chain B_helix 3
EVVEGVFVKSIGSM

SEQ ID NO: 325 - protein, 10 aa, Mus musculus
  ATF4 chain A_helix 1
LTGECKELEK

SEQ ID NO: 326 - protein, 10 aa, Mus musculus
  C/EBP beta chain B_helix 1
ETQHKVLELT

SEQ ID NO: 327 - protein, 8 aa, Mus musculus
  ATF4 chain A_helix 1
LKERADSL

SEQ ID NO: 328 - protein, 8 aa, Mus musculus
  C/EBP beta chain B_helix 1
RLQKKVEQ

SEQ ID NO: 329 - protein, 8 aa, Mus musculus
  ATF4 chain A_helix 1
QYLKDLIE

SEQ ID NO: 330 - protein, 8 aa, Mus musculus
  C/EBP beta chain B_helix 1
LSTLRNLF

SEQ ID NO: 331 - protein, 9 aa, Homo sapiens
  c-Jun chain F_helix 2
KLERIARLE

SEQ ID NO: 332 - protein, 9 aa, Homo sapiens
  c-Fos chain E_helix 2
RELTDTLQA

SEQ ID NO: 333 - protein, 8 aa, Homo sapiens
  c-Jun chain F_helix 2
LKAQNSEL

SEQ ID NO: 334 - protein, 8 aa, Homo sapiens
  c-Fos chain E_helix 2
EDEKSALQ

SEQ ID NO: 335 - protein, 8 aa, Homo sapiens
  c-Jun chain F_helix 2
VAQLKQKV

SEQ ID NO: 336 - protein, 8 aa, Homo sapiens
  c-Fos chain E_helix 2
EKLEFILA

SEQ ID NO: 337 - protein, 10 aa, Domain-Swapped
  Domain-Swapped chain A_helix2
PEELAALESE

SEQ ID NO: 338 - protein, 10 aa, Domain-Swapped
  Domain-Swapped chain B_helix2
GKLAQLKSKL

SEQ ID NO: 339 - protein, 8 aa, Domain-Swapped
  Domain-Swapped chain A_helix2
LEKKLAAL

SEQ ID NO: 340 - protein, 8 aa, Domain-Swapped
  Domain-Swapped chain B_helix2
KKELAQLE

SEQ ID NO: 341 - protein, 8 aa, Saccharomyces cerevisiae
  Gal4 chain A_helix 1
RLERLEQL

SEQ ID NO: 342 - protein, 8 aa, Saccharomyces cerevisiae
  Gal4 chain B_helix 1
SRLERLEQ

SEQ ID NO: 343 - protein, 5 aa, Homo sapiens
  Human Lectin chain A_beta sheet 13
SSFKL

SEQ ID NO: 344 - protein, 5 aa, Homo sapiens
  Human Lectin chain B_beta sheet 13
KLKFS

SEQ ID NO: 345 - protein, 8 aa, Escherichia coli
  Ala-14 chain A_helix
ARANQRAD

SEQ ID NO: 346 - protein, 8 aa, Escherichia coli
  Ala-14 chain B_helix
AARANQRA

SEQ ID NO: 347 - protein, 10 aa, Homo sapiens
  c-Jun chain A_helix
KAQNSELAST

SEQ ID NO: 348 - protein, 10 aa, Homo sapiens
  c-Jun chain B_helix
LKAQNSELAS

SEQ ID NO: 349 - protein, 8 aa, Simian rotavirus A/SA11
  Nsp3 chain A_helix 1
MHSLQNVI

SEQ ID NO: 350 - protein, 8 aa, Simian rotavirus A/SA11
  Nsp3 chain B_helix 1
HSLQNVIP

SEQ ID NO: 351 - protein, 21 aa, Simian rotavirus A/SA12
  Nsp3 chain A_helix 1
ELQVYNNKLERDLQNKIGSLT

SEQ ID NO: 352 - protein, 21 aa, Simian rotavirus A/SA12
  Nsp3 chain B_helix 1
LQVYNNKLERDLQNKIGSLTS

SEQ ID NO: 353 - protein, 13 aa, Rattus norvegicus
  Tpm1 chain A_helix1
IDDLEDELYAQKL

SEQ ID NO: 354 - protein, 13 aa, Rattus norvegicus
  Tpm1 chain B_helix1
DDLEDELYAQKLK

SEQ ID NO: 355 - protein, 8 aa, Bacteriophage P22
  Arc chain A_coil
MPQFNLRW

SEQ ID NO: 356 - protein, 8 aa, Bacteriophage P22
  Arc chain B_coil
WRLNFQPM

SEQ ID NO: 357 - protein, 8 aa, Homo sapiens
  Myc chain A_helix 1
LRKRREQL

SEQ ID NO: 358 - protein, 8 aa, Homo sapiens
  Max chain B_helix 1
KRQNALLE

SEQ ID NO: 359 - protein, 8 aa, Rattus norvegicus
  C/EBPA chain A_helix 1
KVLELTSD

SEQ ID NO: 360 - protein, 8 aa, Rattus norvegicus
  C/EBPA chain B_helix 1
VLELTSDN

SEQ ID NO: 361 - protein, 8 aa, Rattus norvegicus
  C/EBPA chain A_helix 2
EQLSRELD

SEQ ID NO: 362 - protein, 8 aa, Rattus norvegicus
  C/EBPAchain B_helix 2
QLSRELDT

SEQ ID NO: 363 - protein, 5 aa, Laticauda semifasciata
  Erabutoxin chain A_beta sheet 5
LSCCE

SEQ ID NO: 364 - protein, 5 aa, Laticauda semifasciata
  Erabutoxin chain B_beta sheet 5
ECCSL

SEQ ID NO: 365 - protein, 8 aa, Homo sapiens
  Max chain A_helix 1
SFHSLRDS

SEQ ID NO: 366 - protein, 8 aa, Homo sapiens
  Max chain B_helix 2
DKATEYIQ

SEQ ID NO: 367 - protein, 12 aa, Homo sapiens
  Max chain A_helix 2
VHTLQQDIDDLK

SEQ ID NO: 368 - protein, 12 aa, Homo sapiens
  Max chain B_helix 2
HTLQQDIDDLKR

SEQ ID NO: 369 - protein, 8 aa, Homo sapiens
  Max chain A_helix 2
LEQQVRAL

SEQ ID NO: 370 - protein, 8 aa, Homo sapiens
  Max chain B_helix 2
EQQVRALE

SEQ ID NO: 371 - protein, 8 aa, Homo sapiens
  Geminin chain A_helix 1
DNEIARLK

SEQ ID NO: 372 - protein, 8 aa, Homo sapiens
  Geminin chain B_helix 1
NEIARLKK

SEQ ID NO: 373 - protein, 5 aa, Homo sapiens
  Endothelin-1 chain A_beta sheet
RCSCS

SEQ ID NO: 374 - protein, 5 aa, Homo sapiens
  Endothelin-1 chain B_beta sheet
SCSCR

SEQ ID NO: 375 - protein, 8 aa, Homo sapiens
  Cenp-b chain A_helix 1
GEAMAYFA

SEQ ID NO: 376 - protein, 8 aa, Homo sapiens
  Cenp-b chain B_helix 1
AFYAMAEG

SEQ ID NO: 377 - protein, 8 aa, Homo sapiens
  Cenp-b chain A_helix 2
FPIDDRVQ

SEQ ID NO: 378 - protein, 8 aa, Homo sapiens
  Cenp-b chain B_helix 2
KRTVHVLD

SEQ ID NO: 379 - protein, 8 aa, Homo sapiens Mus musculus
  PALS-1-L27N chain A_helix 1
LQVLDRLK

SEQ ID NO: 380 - protein, 8 aa, Homo sapiens Mus musculus
  PATJ-L27 chain B_helix 2
SIDEQSQS

SEQ ID NO: 381 - protein, 27 aa, Salmonella enterica serovar Typhimurium
  TarH chain A_helix 1
ELTSTWDLMLQTRINLSRSAARMMMDA

SEQ ID NO: 382 - protein, 27 aa, Salmonella enterica serovar Typhimurium
  TarH chain B_helix 1
LTSTWDLMLQTRINLSRSAARMMMDAS

SEQ ID NO: 383 - protein, 10 aa, Salmonella enterica serovar Typhimurium
  TarH chain A_helix 1
SELTSTWDLM

SEQ ID NO: 384 - protein, 10 aa, Salmonella enterica serovar Typhimurium
  TarH chain B_helix4
GLAEGLANQM

SEQ ID NO: 385 - protein, 8 aa, Homo sapiens
  Gemin6 chain A_beta sheet 3
LTTDPVSA

SEQ ID NO: 386 - protein, 8 aa, Homo sapiens
  Gemin7 chain B_Helix 1
ALRERYLR

SEQ ID NO: 387 - protein, 7 aa, Homo sapiens
  Gemin6 chain A_beta sheet 5
SMSVTGI

SEQ ID NO: 388 - protein, 7 aa, Homo sapiens
  Gemin7 chain B_beta sheet 7
KFTYSII

SEQ ID NO: 389 - protein, 8 aa, Saccharomyces cerevisiae
  Med7 chain A_helix 1
LKSLLLNY

SEQ ID NO: 390 - protein, 8 aa, Saccharomyces cerevisiae
  Srb7 chain B_helix 2
IQRTKLII

SEQ ID NO: 391 - protein, 8 aa, Saccharomyces cerevisiae
  Med7 chain A_helix 2
IHHLLNEY

SEQ ID NO: 392 - protein, 8 aa, Saccharomyces cerevisiae
  Srb7 chain B_helix 1
ETMQDLCI

SEQ ID NO: 393 - protein, 8 aa, Saccharomyces cerevisiae
  Med7 chain A_helix 3
LEEQLEYK

SEQ ID NO: 394 - protein, 8 aa, Saccharomyces cerevisiae
  Srb7 chain B_helix 3
MLQKKLVE

SEQ ID NO: 395 - protein, 11 aa, Caenorhabditis elegans Homo sapiens
  Lin-7 chain A_helix 1
QRILELMEHVQ

SEQ ID NO: 396 - protein, 11 aa, Caenorhabditis elegans Homo sapiens
  Lin-2 chain B_helix 2
LIRKLEKADNN

SEQ ID NO: 397 - protein, 8 aa, Caenorhabditis elegans Homo sapiens
  Lin-7 chain A_helix 2
NNAKLASL

SEQ ID NO: 398 - protein, 8 aa, Caenorhabditis elegans Homo sapiens
  Lin-2 chain B_helix 1
ELVEKARQ

SEQ ID NO: 399 - protein, 8 aa, Drosophila melanogaster
  DSX chain A_helix 3
MPLMYVIL

SEQ ID NO: 400 - protein, 8 aa, Drosophila melanogaster
  DSX chain B_helix 2
SAEEINAD

SEQ ID NO: 401 - protein, 8 aa, Homo sapiens
  cGMP-dependent protein kinase chain A_helix
EIQELKRK

SEQ ID NO: 402 - protein, 8 aa, Homo sapiens
  cGMP-dependent protein kinase chain A_helix
IQELKRKL

SEQ ID NO: 403 - protein, 8 aa, Homo sapiens
  Usp8 chain A_coil
SVPKELYL

SEQ ID NO: 404 - protein, 8 aa, Homo sapiens
  Usp8 chain B_helix 2
LDRDEERA

SEQ ID NO: 405 - protein, 10 aa, Homo sapiens
  Usp8 chain A_helix2
RDEERAYVLY

SEQ ID NO: 406 - protein, 10 aa, Homo sapiens
  Usp8 chain B_coil
ELYLSSSLKD

SEQ ID NO: 407 - protein, 8 aa, Homo sapiens
  DP1 chain A_helix 1
QNLEVERQ

SEQ ID NO: 408 - protein, 8 aa, Homo sapiens
  E2F1 chain B_helix 1
LEGLTQDL

SEQ ID NO: 409 - protein, 8 aa, Homo sapiens
  DP1 chain A_helix 1
IAFKNLVQ

SEQ ID NO: 410 - protein, 8 aa, Homo sapiens
  E2F1 chain B_helix 1
LRLLSEDT

SEQ ID NO: 411 - protein, 5 aa, Homo sapiens
  DP1 chain A_beta sheet 1
FIIVN

SEQ ID NO: 412 - protein, 5 aa, Homo sapiens
  E2F1 chain B_beta sheet 1
KIVMV

SEQ ID NO: 413 - protein, 19 aa, Homo sapiens
  Beta-myosin S2 chain A_helix 1
EFTRLKEALEKSEARRKEL

SEQ ID NO: 414 - protein, 19 aa, Homo sapiens
  Beta-myosin S2 chain B_helix 1
FTRLKEALEKSEARRKELE

SEQ ID NO: 415 - protein, 9 aa, Homo sapiens
  Beta-myosin S2 chain A_helix 2
LQEKNDLQL

SEQ ID NO: 416 - protein, 9 aa, Homo sapiens
  Beta-myosin S2 chain B_helix 2
QEKNDLQLQ

SEQ ID NO: 417 - protein, 17 aa, Homo sapiens
  Beta-myosin S2 chain A_helix 3
KLEDECSELKRDIDDLE

SEQ ID NO: 418 - protein, 17 aa, Homo sapiens
  Beta-myosin S2 chain B_helix 3
LEDECSELKRDIDDLEL

SEQ ID NO: 419 - protein, 10 aa, Escherichia coli
  Phe-14 chain A_helix
KDDFARFNQR

SEQ ID NO: 420 - protein, 10 aa, Escherichia coli
  Phe-14 chain B_helix
FNAFRSDFQA

SEQ ID NO: 421 - protein, 9 aa, Escherichia coli
  ROM chain A_helix 2
ADEQADICE

SEQ ID NO: 422 - protein, 9 aa, Escherichia coli
  ROM chain B_helix 2
RALCSRYLE

SEQ ID NO: 423 - protein, 11 aa, Haemophilus influenzae
  Hi0947 chain A_helix 1-2
LEKHKAPVDLS

SEQ ID NO: 424 - protein, 11 aa, Haemophilus influenzae
  Hi0947 chain B_helix 1
ELVAIMDNVIA

SEQ ID NO: 425 - protein, 9 aa, Haemophilus influenzae
  Hi0947 chain A_helix 2
SLIALGNMA

SEQ ID NO: 426 - protein, 9 aa, Haemophilus influenzae
  Hi0947 chain B_helix 2
AMNGLAILS

SEQ ID NO: 427 - protein, 11 aa, Haemophilus influenzae
  Hi0947 chain A_helix 3
EALAQAFSNSL

SEQ ID NO: 428 - protein, 11 aa, Haemophilus influenzae
  Hi0947 chain B_helix 3
LSNSFAQALAE

SEQ ID NO: 429 - protein, 5 aa, Arenicola marina lugworm
  Arenicin-2 chain A_beta sheet 1
CVYAY

SEQ ID NO: 430 - protein, 5 aa, Arenicola marina lugworm
  Arenicin-2 chain B_beta sheet 1
VYAYV

SEQ ID NO: 431 - protein, 8 aa, Homo sapiens
  Erbb4 chain A_helix1
ARTPLIAA

SEQ ID NO: 432 - protein, 8 aa, Homo sapiens
  Erbb4 chain B_helix1
RTPLIAAG

SEQ ID NO: 433 - protein, 8 aa, Homo sapiens
  FGFR3 chain A_helix 1
AGSVYAGI

SEQ ID NO: 434 - protein, 8 aa, Homo sapiens
  FGFR3 chain B_helix 1
EAGSVYAG

SEQ ID NO: 435 - protein, 5 aa, Homo sapiens
  Xcl1 chain A_beta sheet 1
CVSLT

SEQ ID NO: 436 - protein, 5 aa, Homo sapiens
  Xcl1 chain B_beta sheet 1
TLSVC

SEQ ID NO: 437 - protein, 5 aa, Homo sapiens
  Xcl1 chain A_beta sheet 2
TYTIT

SEQ ID NO: 438 - protein, 5 aa, Homo sapiens
  Xcl1 chain B_beta sheet 2
TITYT

SEQ ID NO: 439 - protein, 8 aa, Homo sapiens
  CXCL12 chain A_beta sheet 1
VKHLKILN

SEQ ID NO: 440 - protein, 8 aa, Homo sapiens
  CXCL12 chain B_beta sheet 1
NLIKLHKV

SEQ ID NO: 441 - protein, 10 aa, Homo sapiens
  CXCL12 chain A_helix1
IQEYLEKALN

SEQ ID NO: 442 - protein, 10 aa, Homo sapiens
  CXCL12 chain B_helix1
NLAKELYEQI

SEQ ID NO: 443 - protein, 19 aa, Staphylococcus aureus subsp. aureus MW2
  Ylan chain A_helix 2
EVLDTQMFGLQKEVDFAVK

SEQ ID NO: 444 - protein, 19 aa, Staphylococcus aureus subsp. aureus MW2
  Ylan chain B_helix 2
LYEEVLDTQMFGLQKEVDF

SEQ ID NO: 445 - protein, 8 aa, Staphylococcus aureus subsp. aureus MW2
  Ylan chain A_helix 1
QLTKDADE

SEQ ID NO: 446 - protein, 8 aa, Staphylococcus aureus subsp. aureus MW2
  Ylan chain B_helix 2
LKVAFDVE

SEQ ID NO: 447 - protein, 8 aa, Arabidopsis thaliana
  Hy5 chain A_helix
GSAYLSEL

SEQ ID NO: 448 - protein, 8 aa, Arabidopsis thaliana
  Hy5 chain B_helix
SAYLSELE

SEQ ID NO: 449 - protein, 8 aa, Arabidopsis thaliana
  Hy5 chain A_helix
LENKNSEL

SEQ ID NO: 450 - protein, 8 aa, Arabidopsis thaliana
  Hy5 chain B_helix
ENKNSELE

SEQ ID NO: 451 - protein, 8 aa, Arabidopsis thaliana
  Hy5 chain A_helix
LEERLSTL

SEQ ID NO: 452 - protein, 8 aa, Arabidopsis thaliana
  Hy5 chain B_helix
EERLSTLQ

SEQ ID NO: 453 - protein, 8 aa, Mus musculus
  E47 helix 2
QVILGLEQ

SEQ ID NO: 454 - protein, 8 aa, Mus musculus
  NeuroD1 helix 2
KNYIWALS

SEQ ID NO: 455 - protein, 8 aa, Mus musculus
  E47 chain A_helix 1
EAFRELGR

SEQ ID NO: 456 - protein, 8 aa, Mus musculus
  NeuroD1 chain B_helix 2
LAKNYIWA

SEQ ID NO: 457 - protein, 8 aa, Mus musculus
  E47 chain A_helix 2
ILQQAVQV

SEQ ID NO: 458 - protein, 8 aa, Mus musculus
  NeuroD1 chain B_helix 1
NAALDNLR

SEQ ID NO: 459 - protein, 9 aa, Mus musculus
  c-Fos chain A_helix 1
LEDEKSALQ

SEQ ID NO: 460 - protein, 9 aa, Mus musculus
  MafB chain B_helix 1
QLIQQVEQL

SEQ ID NO: 461 - protein, 8 aa, Homo sapiens
  Bst2 chain A_helix1
HKLQDASA

SEQ ID NO: 462 - protein, 8 aa, Homo sapiens
  Bst2 chain B_helix1
KLQDASAE

SEQ ID NO: 463 - protein, 8 aa, Homo sapiens
  CHMP3 chain R_helix 1
SRLATLRS

SEQ ID NO: 464 - protein, 8 aa, Homo sapiens
  STAMBP chain B_helix 3
SGLQSLAR

SEQ ID NO: 465 - protein, 8 aa, Homo sapiens
  SCL chain A_helix 2
AFAELRKL

SEQ ID NO: 466 - protein, 8 aa, Homo sapiens
  E47 chain B_helix 2
LILQQAVQ

SEQ ID NO: 467 - protein, 9 aa, Homo sapiens
  SCL chain A_helix 2
NEILRLAMK

SEQ ID NO: 468 - protein, 9 aa, Homo sapiens
  E47 chain B_helix 2
DINEAFREL

SEQ ID NO: 469 - protein, 8 aa, Saccharomyces cerevisiae
  GCN4 chain A_helix 2
QLEDKVEE

SEQ ID NO: 470 - protein, 8 aa, Saccharomyces cerevisiae
  GCN4 chain B_helix 2
LEDKVEEL

SEQ ID NO: 471 - protein, 10 aa, Saccharomyces cerevisiae
  GCN4 chain A_helix 2
LENEVARLKK

SEQ ID NO: 472 - protein, 10 aa, Saccharomyces cerevisiae
  GCN4 chain B_helix 2
ENEVARLKKL

SEQ ID NO: 473 - protein, 8 aa, Homo sapiens
  HV1 chain A_helix1
LKQMNVQL

SEQ ID NO: 474 - protein, 8 aa, Homo sapiens
  HV1 chain B_helix1
KQMNVQLA

SEQ ID NO: 475 - protein, 8 aa, Cyanobacterium Cyanothece
  Cce_0567 chain A_helix 1
KVRKLNSK

SEQ ID NO: 476 - protein, 8 aa, Cyanobacterium Cyanothece
  Cce_0567 chain B_helix 1
LTEEWINL

SEQ ID NO: 477 - protein, 8 aa, Cyanobacterium Cyanothece
  Cce_0567 chain A_helix 1
LHDLAEGL

SEQ ID NO: 478 - protein, 8 aa, Cyanobacterium Cyanothece
  Cce_0567 chain B_helix 1
ERFIEYTK

SEQ ID NO: 479 - protein, 12 aa, Helicobacter pylori
  HP0062 chain A_helix 1
EVREFVGHLERF

SEQ ID NO: 480 - protein, 12 aa, Helicobacter pylori
  HP0062 chain B_helix 1
LNHFHNSLSNVE

SEQ ID NO: 481 - protein, 11 aa, Helicobacter pylori
  HP0062 chain A_helix 2
RDKFSEVLDNL

SEQ ID NO: 482 - protein, 11 aa, Helicobacter pylori
  HP0062 chain B_helix 2
AIQEQAAEDFE

SEQ ID NO: 483 - protein, 10 aa, Enterobacter sp. RFL1396
  C.esp1396i chain A_helix 5
VVFFEMLIKE

SEQ ID NO: 484 - protein, 10 aa, Enterobacter sp. RFL1396
  C.esp1396i chain B_helix 5
IEKILMEFFV

SEQ ID NO: 485 - protein, 16 aa, Homo sapiens
  MAPRE1 chain A_helix 1
ELMQQVNVLKLTVEDL

SEQ ID NO: 486 - protein, 16 aa, Homo sapiens
  MAPRE1 chain B_helix 1
LMQQVNVLKLTVEDLE

SEQ ID NO: 487 - protein, 8 aa, Homo sapiens
  MAPRE1 chain A_helix 1
FGKLRNIE

SEQ ID NO: 488 - protein, 8 aa, Homo sapiens
  MAPRE1 chain B_helix 1
GKLRNIEL

SEQ ID NO: 489 - protein, 8 aa, Caenorhabditis elegans
  Gld1 chain A_helix 1
EYLADLVK

SEQ ID NO: 490 - protein, 8 aa, Caenorhabditis elegans
  Gld1 chain B_helix 2
LREVNSFM

SEQ ID NO: 491 - protein, 15 aa, HIV type 1 HXB3 ISOLATE
  Rev chain A_helix 1
DEDSLKAVRLIKFLY

SEQ ID NO: 492 - protein, 15 aa, HIV type 1 HXB3 ISOLATE
  Rev chain B_helix 1
YLFKILRVAKLSDED

SEQ ID NO: 493 - protein, 5 aa, Helicobacter Pylori
  MinE chain A_beta sheet 1
LKLIL

SEQ ID NO: 494 - protein, 5 aa, Helicobacter Pylori
  MinE chain B_beta sheet 1
ALILK

SEQ ID NO: 495 - protein, 18 aa, Homo sapiens
  Pkg1-Beta chain A_helix
IDELELELDQKDELIQML

SEQ ID NO: 496 - protein, 18 aa, Homo sapiens
  Pkg1-Beta chain B_helix
DELELELDQKDELIQMLQ

SEQ ID NO: 497 - protein, 16 aa, Schizosaccharomyces pombe
  Swi5 chain A_helix
QDALAKLKNRDAKQTV

SEQ ID NO: 498 - protein, 16 aa, Schizosaccharomyces pombe
  Swi5chain B_helix
LAIDRIENYTHLLDIH

SEQ ID NO: 499 - protein, 16 aa, Schizosaccharomyces pombe
  Swi5 chain A_helix
KEQLESSLQDALAKLK

SEQ ID NO: 500 - protein, 16 aa, Schizosaccharomyces pombe
  Swi5chain C_helix
KLKALADQLSSELQEK

SEQ ID NO: 501 - protein, 13 aa, Schizosaccharomyces pombe
  Swi5 chain B_helix
VQKHIDLLHTYNE

SEQ ID NO: 502 - protein, 13 aa, Schizosaccharomyces pombe
  Swi5chain C_helix
HLLEQQKEQLESS

SEQ ID NO: 503 - protein, 8 aa, Mus musculus
  Hv1 chain A_helix 1
LKQINIQL

SEQ ID NO: 504 - protein, 8 aa, Mus musculus
  Hv1 chain B_helix 1
KQINIQLA

SEQ ID NO: 505 - protein, 10 aa, Saccharomyces cerevisiae
  Sgt2 chain A_helix 1
EIAALIVNYF

SEQ ID NO: 506 - protein, 10 aa, Saccharomyces cerevisiae
  Sgt2 chain B_helix 1
FYNVILAAIE

SEQ ID NO: 507 - protein, 16 aa, Saccharomyces cerevisiae
  Sgt2 chain A_helix 2
ADSLNVAMDCISEAFG

SEQ ID NO: 508 - protein, 16 aa, Saccharomyces cerevisiae
  Sgt2 chain B_helix 1
GFAESICDMAVNLSDA

SEQ ID NO: 509 - protein, 9 aa, Homo sapiens
  Cc2-LZ chain A_helix 1
QLEDLKQQL

SEQ ID NO: 510 - protein, 9 aa, Homo sapiens
  Cc2-LZ chain B_helix 1
LEDLKQQLQ

SEQ ID NO: 511 - protein, 17 aa, Homo sapiens
  Cc2-LZ chain A_helix 2
ELLQEQLEQLQREYSKL

SEQ ID NO: 512 - protein, 17 aa, Homo sapiens
  Cc2-LZ chain B_helix 2
LLQEQLEQLQREYSKLK

SEQ ID NO: 513 - protein, 8 aa, Mus musculus
  Qua1 chain A_helix 2
  VARIANT (6)...(6): Xaa = any amino acid
TPDYLXQL

SEQ ID NO: 514 - protein, 8 aa, Mus musculus
  Qua1 chain B_helix 2
RSIEEDLL

SEQ ID NO: 515 - protein, 10 aa, Homo sapiens
  DD_Ribeta_PKA chain A_helix3
KFLREHFEKL

SEQ ID NO: 516 - protein, 10 aa, Homo sapiens
  DD_Ribeta_PKA chain B_helix3
LKEFHERLKK

SEQ ID NO: 517 - protein, 16 aa, Homo sapiens
  Trim25 chain A_helix1
SADLEATLRHKLTVMY

SEQ ID NO: 518 - protein, 16 aa, Homo sapiens
  Trim25 chain B_helix1
DRKTLSQEIEEKLTQI

SEQ ID NO: 519 - protein, 8 aa, Homo sapiens
  Trim25 chain A_helix1
LDDVRNRQ

SEQ ID NO: 520 - protein, 8 aa, Homo sapiens
  Trim25 chain B_helix1
YITDFKSN

SEQ ID NO: 521 - protein, 13 aa, Homo sapiens
  Trim25 chain A_helix1
LRHKLTVMYSQIN

SEQ ID NO: 522 - protein, 13 aa, Homo sapiens
  Trim25 chain B_helix2
KASKLRGISTKPV

SEQ ID NO: 523 - protein, 8 aa, Homo sapiens
  Trim25 chain A_helix1
VRNRQQDV

SEQ ID NO: 524 - protein, 8 aa, Homo sapiens
  Trim25 chain B_helix2
HKLIKGIH

SEQ ID NO: 525 - protein, 13 aa, Homo sapiens
  Trim25 chain A_helix1
RKVEQLQQEYTEM

SEQ ID NO: 526 - protein, 13 aa, Homo sapiens
  Trim25 chain B_helix2
LKNELKQCIGRLQ

SEQ ID NO: 527 - protein, 10 aa, Homo sapiens
  Trim25 chain A_helix2
KNELKQCIGR

SEQ ID NO: 528 - protein, 10 aa, Homo sapiens
  Trim25 chain B_helix2
GICQKLENKL

SEQ ID NO: 529 - protein, 8 aa, Homo sapiens
  Mst1 chain A_helix Rassf5
LQKRLLAL

SEQ ID NO: 530 - protein, 8 aa, Homo sapiens
  Sarah chain B_helix
RLAEELKQ

SEQ ID NO: 531 - protein, 5 aa, Homo sapiens
  Naf1 chain A_beta sheet 2
PLILK

SEQ ID NO: 532 - protein, 5 aa, Homo sapiens
  Naf1 chain B_coil
VVNEI

SEQ ID NO: 533 - protein, 9 aa, Mus musculus
  NEMOchain A_helix 1
QLEDLRQQL

SEQ ID NO: 534 - protein, 9 aa, Mus musculus
  NEMO chain B_helix 1
LEDLRQQLQ

SEQ ID NO: 535 - protein, 8 aa, Mus musculus
  NEMOchain A_helix 1
KQELIDKL

SEQ ID NO: 536 - protein, 8 aa, Mus musculus
  NEMO chain B_helix 1
QELIDKLK

SEQ ID NO: 537 - protein, 8 aa, Mus musculus
  NEMOchain A_helix 2
LKAQADIY

SEQ ID NO: 538 - protein, 8 aa, Mus musculus
  NEMO chain B_helix 2
KAQADIYK

SEQ ID NO: 539 - protein, 26 aa, Mus musculus
  NEMOchain A_helix 2-3
AREKLVEKKEYLQEQLEQLQREFNKL

SEQ ID NO: 540 - protein, 26 aa, Mus musculus
  NEMO chain B_helix 2-3
REKLVEKKEYLQEQLEQLQREFNKLK

SEQ ID NO: 541 - protein, 8 aa, Homo sapiens
  GBR1 chain A_helix 1
KSRLLEKE

SEQ ID NO: 542 - protein, 8 aa, Homo sapiens
  GBR2 chain B_helix 1
SRLEGLQS

SEQ ID NO: 543 - protein, 12 aa, Homo sapiens
  GBR1 chain A_helix 1
EERVSELRHQLQ

SEQ ID NO: 544 - protein, 12 aa, Homo sapiens
  GBR2 chain B_helix 1
LDKDLEEVTMQL

SEQ ID NO: 545 - protein, 8 aa, Homo sapiens
  Jip3 chain A_helix 1
DLIAKVDQ

SEQ ID NO: 546 - protein, 8 aa, Homo sapiens
  Jip3 chain B_helix 1
IRNELKVK

SEQ ID NO: 547 - protein, 9 aa, Homo sapiens
  Pkg1-Alpha chain A_helix
LKRKLHKLQ

SEQ ID NO: 548 - protein, 9 aa, Homo sapiens
  Pkg1-Alpha chain B_helix
ELKRKLHKL

SEQ ID NO: 549 - protein, 8 aa, Homo sapiens
  VBP chain A_helix
EIRAAFLE

SEQ ID NO: 550 - protein, 8 aa, Homo sapiens
  VBP chain B_helix
LEIRAAFL

SEQ ID NO: 551 - protein, 5 aa, Homo sapiens
  NBL1 chain A_beta sheet 3
GQCFS

SEQ ID NO: 552 - protein, 5 aa, Homo sapiens
  NBL1 chain B_beta sheet 3
SFCQG

SEQ ID NO: 553 - protein, 17 aa, Homo sapiens
  Gp7-Myh7-EB1 chain A_helix 3
KLEKEKSEFKLELDDVT

SEQ ID NO: 554 - protein, 17 aa, Homo sapiens
  Gp7-Myh7-EB1 chain B_helix 3
LEKEKSEFKLELDDVTS

SEQ ID NO: 555 - protein, 9 aa, Homo sapiens
  Gp7-Myh7-EB1 chain A_helix 3
ELGEQIDNL

SEQ ID NO: 556 - protein, 9 aa, Homo sapiens
  Gp7-Myh7-EB1 chain B_helix 3
LGEQIDNLQ

SEQ ID NO: 557 - protein, 9 aa, Homo sapiens
  Gp7-Myh7-EB1 chain A_helix 2
LQQLRVNYG

SEQ ID NO: 558 - protein, 9 aa, Homo sapiens
  Gp7-Myh7-EB1 chain B_helix 2
QQLRVNYGS

SEQ ID NO: 559 - protein, 8 aa, Homo sapiens
  Gp7-Myh7-EB1 chain A_helix 2
TEALQQLR

SEQ ID NO: 560 - protein, 8 aa, Homo sapiens
  Gp7-Myh7-EB1 chain B_helix 1
LIDEHEEP

SEQ ID NO: 561 - protein, 14 aa, Ixodes scapularis
  Sialostatin L chain A_coil+beta sheet 1&2
VETQVVAGTNYRLT

SEQ ID NO: 562 - protein, 14 aa, Ixodes scapularis
  Sialostatin L chain A_coil+beta sheet 1&2
TLRYNTGAVVQTEV

SEQ ID NO: 563 - protein, 5 aa, Homo sapiens
  Norrin chain A_beta sheet 3
ASRSE

SEQ ID NO: 564 - protein, 5 aa, Homo sapiens
  Norrin chain B_beta sheet 2
GECRA

SEQ ID NO: 565 - protein, 15 aa, Mus musculus
  Kinesin-like Protein chain A_helix1
LKEKLEESEKLIKEL

SEQ ID NO: 566 - protein, 15 aa, Mus musculus
  Kinesin-like Protein chain B_helix1
ELKEKLEESEKLIKE

SEQ ID NO: 567 - protein, 12 aa, Mus musculus
  Kinesin-like Protein chain A_helix1
LESMGISLETSG

SEQ ID NO: 568 - protein, 12 aa, Mus musculus
  Kinesin-like Protein chain B_helix1
QLESMGISLETS

SEQ ID NO: 569 - protein, 8 aa, Mus musculus
  Cc1-fha chain A_helix 1
LKEKLEES

SEQ ID NO: 570 - protein, 8 aa, Mus musculus
  Cc1-fha chain B_helix 1
ELKEKLEE

SEQ ID NO: 571 - protein, 8 aa, Homo sapiens
  Phenylalanine-4-hydroxylase chain A_helix1
ALAKVLRL

SEQ ID NO: 572 - protein, 8 aa, Homo sapiens
  Phenylalanine-4-hydroxylase chain A_helix1
FLRLVKAL

SEQ ID NO: 573 - protein, 5 aa, Acinetobacter phage AP205
  Phage Coat Protein chain A_beta sheet 5
IRTVI

SEQ ID NO: 574 - protein, 5 aa, Acinetobacter phage AP205
  Phage Coat Protein chain A_beta sheet 5
VTRIS

SEQ ID NO: 575 - protein, 8 aa, Bos taurus
  Myosin X chain A_helix 2
SLQKLQQL

SEQ ID NO: 576 - protein, 8 aa, Bos taurus
  Myosin X chain C_helix 3
VEEILRLE

SEQ ID NO: 577 - protein, 9 aa, Bos taurus
  Myosin X chain A_helix 2
LEKEIEDLQ

SEQ ID NO: 578 - protein, 9 aa, Bos taurus
  Myosin X chain C_helix 2
QLDEIEKEL

SEQ ID NO: 579 - protein, 17 aa, Pelecanus crispus Bruch, 1832
  BLM Helicase chain A_helix 1
EQQLYAVMDDICKLVDA

SEQ ID NO: 580 - protein, 17 aa, Pelecanus crispus Bruch, 1832
  BLM Helicase chain A_helix 2
ALLKRRLGRQLLLEKAC

SEQ ID NO: 581 - protein, 10 aa, Drosophila melanogaster
  Ncd chain A_helix1
AELETCKEQL

SEQ ID NO: 582 - protein, 10 aa, Drosophila melanogaster
  Ncd chain B_helix1
ELETCKEQLF